(19) United States
(12) Patent Application Publication
(10) Pub. No.: US 2024/0207410 A1
(43) Pub. Date: Jun. 27, 2024

(54) STABILIZED LIQUID COMPOSITIONS COMPRISING A LEVODOPA-TYROSINE CONJUGATE AND USES THEREOF

(71) Applicant: NeuroDerm, Ltd., Rehovot (IL)

(72) Inventors: Avital Laxer, Rehovot (IL); Elana Gazal, Rehovot (IL); Mazzi Dagan-Lion, Rehovot (IL); Irena Vainshtok, Rehovot (IL); Alaa Talhami, Rehovot (IL); Alex Mainfield, Rehovot (IL); Eduardo Zawoznik, Mazkeret Batya (IL); Kenji Morokuma, Osaka (JP); Kazuki Nakayama, Osaka (JP); Eiji Yoshida, Osaka (JP); Akira Nakao, Osaka (JP)

(21) Appl. No.: 18/549,452

(22) PCT Filed: Mar. 9, 2022
(86) PCT No.: PCT/IL2022/050269

§ 371 Date: Sep. 7, 2023

Related U.S. Application Data
(60) Provisional application No. 63/296,032, filed on Jan. 3, 2022.
(60) Provisional application No. 63/159,236, filed on Mar. 10, 2021.

(51) Int. Cl. A61K 47/54; A61K 31/198; A61K 47/10; A61K 47/18; A61K 47/20; A61K 47/22

(52) U.S. Cl. CPC A61K 47/542; A61K 31/198; A61K 47/10; A61K 47/18; A61K 47/183; A61K 47/20; A61K 47/22

(57) ABSTRACT

Disclosed herein are levodopa prodrug compounds and methods for their use. Further disclosed herein are liquid pharmaceutical formulations comprising a levodopa-tyrosine conjugate and a stabilizer, wherein the liquid pharmaceutical formulations may further comprise a decarboxylase inhibitor, such as carbidopa, an antioxidant, a solvent, or any other pharmaceutically acceptable excipient. Further disclosed are methods of treating generative conditions and/or conditions characterized by reduced levels of dopamine in the brain, such as Parkinson's disease, comprising administering the disclosed prodrugs and/or liquid pharmaceutical formulations.

### CROSS REFERENCE TO RELATED APPLICATIONS

This application claims the benefit of and priority to U.S. Provisional Patent Application No. 63/159,236, filed Mar. 10, 2021 and U.S. Provisional Patent Application No. 63/296,032, filed Jan. 3, 2022, the entire disclosure of each of which is hereby incorporated by reference in its entirety.

### TECHNICAL FIELD

The present invention relates to a levodopa prodrug compound and its pharmaceutical use. The present invention also is directed to stabilized compositions comprising a levodopa-tyrosine conjugate (LD-Tyr) and salts thereof, methods of preparing LD-Tyr compositions, and methods of using the same in, for example, the treatment of conditions characterized by neurodegeneration and/or reduced levels of dopamine in the brain, e.g., Parkinson's disease.

### BACKGROUND

Parkinson's disease is a degenerative condition characterized by reduced concentration of the neurotransmitter dopamine in the brain. Levodopa (L-dopa or L-3,4-dihydroxyphenylalanine) is an immediate metabolic precursor of dopamine that, unlike dopamine, is able to cross the blood brain barrier, and is most commonly used for restoring the dopamine concentration in the brain. For the past 40 years, levodopa has remained the most effective therapy for the treatment of Parkinson's disease.

However, conventional treatments for Parkinson's disease with levodopa have proven to be inadequate for many reasons of record in the medical literature. For example, some patients eventually become less responsive to levodopa, such that previously effective doses eventually fail to produce any therapeutic benefit. Thus, the systemic administration of levodopa, while producing clinically beneficial effects at first, is complicated by the need to increase the doses to such high doses that may result in adverse side effects. For such reasons, the benefits of levodopa treatment often begin to diminish after about 3 or 4 years of therapy, irrespective of the initial therapeutic response.

The peripheral administration of levodopa is further complicated by the fact that only about 1-3% of the levodopa administered is able to enter the brain unaltered, wherein most of the levodopa is metabolized extracerebrally, predominantly by the decarboxylation of the levodopa to dopamine, which does not penetrate the blood brain barrier and therefore, is ineffective in treatment. The metabolic transformation of levodopa to dopamine is catalyzed by the aromatic L-amino acid decarboxylase enzyme, an ubiquitous enzyme with particularly high concentrations in the intestinal mucosa, liver, brain and brain capillaries. Due to the possibility of extracerebral metabolism of levodopa, it is necessary to administer large doses of levodopa, leading to high extracerebral concentrations of dopamine. The co-administration of levodopa and a peripheral dopamine decarboxylase (aromatic L-amino acid decarboxylase) inhibitor, such as carbidopa or benserazide, has been found to reduce the dosage requirements of levodopa and, respectively, some of the side effects; however, frequently, the obtained reduction is insufficient.

Finally, certain fluctuations in the clinical response to levodopa occur with increasing frequency with prolonged treatment. In some patients, these fluctuations relate to the timing of levodopa intake, known as “wearing-off reactions” or “end-of-dose akinesia”. In other instances, fluctuations in the clinical state are unrelated to the timing of doses and are generally referred to as “on-off phenomenon”. In the on-off phenomenon, “off-periods” of marked akinesia and bradykinesia alternate over the course of a few hours with “on-periods” of improved mobility, which are often associated with troublesome dyskinesia.

To maintain a desired dopamine concentration in the brain, a method for administering a formulation containing levodopa with a pump continuously has been developed. As an example, a method for continuous intestinal delivery of an L-dopa/carbidopa gel (known under the trade name DuoDopa® in Europe and Duopa® in the United States) is known, and treatment for Parkinson's disease has been performed using this method. However, since intestinal insertion is required, burden on the patient is large, and thus, a less invasive and pharmacokinetically steady delivery system is demanded. An example of a less invasive delivery system is a method for subcutaneously administering a solution formulation with a pump. Such a formulation is currently under study, but has not yet reached the market.

As described above, when developing an optimal formulation for a continuous delivery system in the treatment of Parkinson's disease, it is necessary that an active ingredient is soluble and stable in the formulation. Many different approaches can be considered; however, to improve the solubility of the active ingredient itself, one approach may be the creation of a novel levodopa prodrug. For example, a prodrug in which an amino acid is added to a levodopa molecule is known (U.S. Pat. No. 3,803,120). In addition, a levodopa prodrug compound having phosphoric acid ester is also known (International Patent Publication No. WO2017/184871).

Further, it is well accepted in the art that many of the disadvantages referred to above result from the unfavorable pharmacokinetic properties of levodopa and, more particularly, from its poor water solubility, bioavailability and fast degradation in vivo. Another approach may be to develop effective therapeutic formulations with improved stability for treating disorders such as Parkinson's disease.

Amino acids, which contain both amino and carboxylic groups, are the basic unit of proteins. Generally, amino acids are known to play a major role in the body, being involved in tissue protein formation and enzyme hormone formation. Therefore, any deficiency in amino acids affects protein synthesis. Amino acids are also known to regulate processes related to gene expression and further, amino acids modulate the protein function involved in messenger RNA translation. Several amino acids, such as tyrosine, are synthesized in the human body, while others, known as essential amino acids, such as arginine and lysine, are consumed by diet. The lanthionine amino acid is a natural, but non-proteinogenic, diamino diacid, and is structurally related to the amino acid cysteine. Lanthionine has a central monosulfur moiety bound to two alanine residues (R/S configuration), allowing the possibility of different stereometric forms of lanthionine.

Amino acids are ionized in aqueous solutions, wherein the pH of the solution affects the ionic species of the amino acid and determines whether the amino acid will be in the form of a zwitterion, cation or anion. The permeability coefficients of the various compounds through the skin is dependent on their ionic form, wherein non-ionized species generally have higher permeability coefficients in comparison to ionized species and further, cations generally have higher permeability coefficients than anions.

U.S. Pat. Nos. 3,803,120, 4,035,507, 5,686,423 and U.S. Patent Application No. 2002/099013 disclose certain levodopa amino acid and levodopa peptide conjugates; however, details regarding formulations are not provided therein, and when provided, only solid oral formulations are contemplated. The theoretical option of preparing liquid compositions is briefly mentioned in U.S. Pat. No. 3,803,120 (US '120, column 3, lines 49-53); however, no such compositions were prepared and moreover, it is erroneously disclosed that the conjugates are soluble (column 3, lines 65-66).

Further, levodopa amino acid conjugates, such as LD-Tyr, can be unstable and/or form impurities over time. For example, LD-Tyr has a propensity to form a diketopiperazine (DKP) impurity as shown in the scheme below.

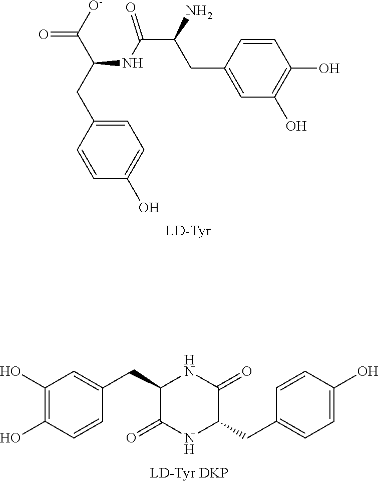 [SMILES: N[C@@H](Cc1ccc(O)c(O)c1)C(=O)N[C@@H](Cc1ccc(O)cc1)C(=O)[O-] ; O=C1N[C@H](Cc2ccc(O)c(O)c2)C(=O)N[C@H]1Cc1ccc(O)cc1]

As detailed above, there is still a need for effective, stable formulations, and particularly liquid formulations, for treating disorders such as Parkinson's disease.

### SUMMARY OF THE INVENTION

The present disclosure is intended to provide a novel compound that, by creating novel prodrug, has improved solubility and stability in solution compared to levodopa and allows the compound to be converted into levodopa in the body.

As a result of intensive studies to solve the above problem, the present inventors have found that a levodopa prodrug compound represented by a general formula (I) or (III) has a high levodopa conversion efficiency and has a good solubility and stability in solution, and thus have accomplished the present invention.

Accordingly, in one embodiments, the disclosure relates to levodopa amino acid complex represented by the following formula (I) or (III) or a pharmaceutically acceptable salt thereof:

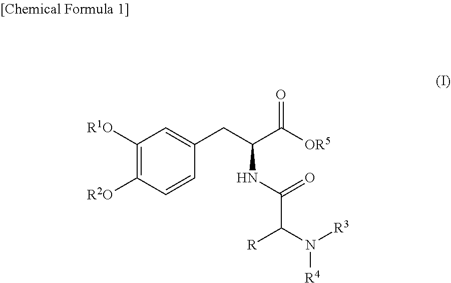

wherein R is an amino acid side chain that may be substituted;
R¹ and R² may be the same or different, and are each independently a hydrogen atom, a C₁-C₆ alkyl, a C₁-C₆ alkanoyl, a phosphono, a sulfino, or a glycosyl that may be substituted, provided that R¹ and R² are not hydrogen atom at the same time;
R³ and R⁴ may be the same or different, and are each independently a hydrogen atom or a C₁-C₆ alkyl; and
R⁵ is a hydrogen atom, or

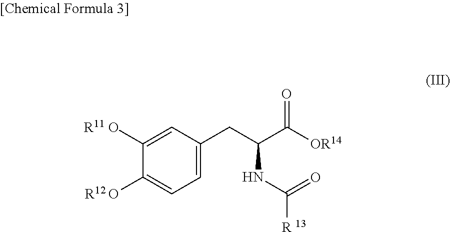

wherein R¹¹ and R¹² are the same or different and are each a hydrogen, an alkyl, an alkanoyl, a P(═O)(OH)₂, S(═O)(OH) or a glycosyl that may be substituted;
R¹³ is an alkyl that may be substituted, —R¹⁵—O—R¹⁶ or a 5-membered heterocyclyl containing at least one nitrogen atom, wherein R¹⁵ is an alkylene, and R¹⁶ is a hydrogen, an alkyl, P(═O)(OH)₂, S(═O)(OH), or a glycosyl that may be substituted; and
R¹⁴ is a hydrogen or an alkyl,
provided that following compounds are excluded;
(2S)-2-[(2-aminoacetyl)amino]-3-(3,4-diacetyloxyphenyl)propanoic acid,
(2S)-2-[[(2S)-2-amino-6-[(2-chlorophenyl)methoxycarbonylamino]hexanoyl]amino]-3-(3,4-dimethoxyphenyl)propanoic acid,
(2S)-2-[[(2S)-2-amino-3-(3,4-dihydroxyphenyl)propanoyl]amino]-3-(4-hydroxy-3-methoxyphenyl)propanoic acid,
(2S)-2-[[(2S)-2-amino-3-phenylpropanoyl]amino]-3-(3,4-dimethoxyphenyl)propanoic acid,
(2S)-2-[[(2R)-2-amino-3-phenylpropanoyl]amino]-3-(3,4-diacetyloxyphenyl)propanoic acid, and
(2S)-2-[[(2S)-2-amino-5-methoxy-5-oxopentanoyl]amino]-3-(3,4-dimethoxyphenyl)propanoic acid.

In certain embodiments, the disclosure further relates to the levodopa amino acid complex according to the above (1) or a pharmaceutically acceptable salt thereof, wherein R³, R⁴ and R⁵ are hydrogen atoms,
R¹ and R² are the same or different, and are each a hydrogen atom, an acetyl or a phosphono, provided that R¹ and R² are not hydrogen atom at the same time.

In certain embodiments, the disclosure further relates to the levodopa amino acid complex according to the above (1) or (2) or a pharmaceutically acceptable salt thereof, wherein R³, R⁴ and R⁵ are hydrogen atoms,
R¹ is a hydrogen atom, and
R² is a phosphono.

In certain embodiments, the disclosure further relates to the levodopa amino acid complex according to any one of the above embodiments, or a pharmaceutically acceptable salt thereof, wherein an amino acid of the amino acid side chain is a glutamic acid, valine, alanine, lysine, 3,4-dihydroxyphenylalanine or tyrosine.

In certain embodiments, the disclosure further relates to the levodopa amino acid complex selected from the group consisting of:
(2S)-2-[[(2S)-2-amino-3-phosphonooxypropanoyl]amino]-3-(3,4-dihydroxyphenyl)propanoic acid,
(2S)-2-[[(2S)-2-amino-3-(4-phosphonooxyphenyl)propanoyl]amino]-3-(3,4-dihydroxyphenyl)propanoic acid,
(2S)-2-amino-5-[[(1S)-1-carboxy-2-(3,4-diacetoxyphenyl)ethyl]amino]-5-oxo-pentanoic acid,
(2S)-3-(3,4-dihydroxyphenyl)-2-[(2-methyl-2-phosphonooxypropanoyl)amino]propanoic acid, and
(2S)-2-[[(2S)-2-amino-3-[4-[(2S,3R,4S,5S,6R)-3,4,5-trihydroxy-6-(hydroxymethyl)oxan-2-yl]oxyphenyl]propanoyl]amino]-3-(3,4-dihydroxyphenyl)propanoic acid.

In certain embodiments, the disclosure further relates to a liquid pharmaceutical composition comprising the levodopa amino acid complex according to any one of the above embodiments or a pharmaceutically acceptable salt thereof as an active ingredient.

In certain embodiments, the disclosure further relates to therapeutic agent for a neurodegenerative disease and/or a disease or symptom caused by a decrease in dopamine concentration in the brain, the therapeutic agent comprising the levodopa amino acid complex according to any one of the above embodiments or a pharmaceutically acceptable salt thereof as an active ingredient. In certain embodiments, the neurodegenerative disease and/or the disease or symptom caused by a decrease in dopamine concentration in the brain is Parkinson's disease.

Further provided herein, inter alia, are compositions comprising levodopa-tyrosine conjugates (LD-Tyr) or salts thereof (e.g., pharmaceutically acceptable salts thereof), for example, pharmaceutically acceptable compositions, for example, liquid pharmaceutical compositions, with improved stability. Also described herein are methods of preparing such compositions. Also disclosed are methods of using compositions comprising LD-Tyr and pharmaceutically acceptable salts thereof, and compositions comprising the same in, for example, the treatment of conditions characterized by neurodegeneration and/or reduced levels of dopamine in the brain, e.g., Parkinson's disease.

Disclosed herein is a liquid pharmaceutical composition comprising:
a levodopa-tyrosine (LD-Tyr) conjugate of the formula (II):

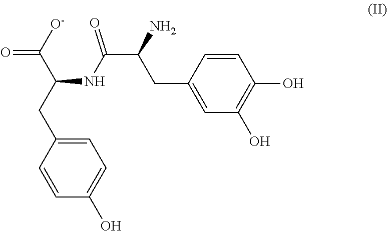 [SMILES: N[C@@H](Cc1ccc(O)c(O)c1)C(=O)N[C@@H](Cc1ccc(O)cc1)C(=O)[O-]]

an enantiomer, diastereomer, racemate, ion, zwitterion, pharmaceutically acceptable salt thereof, or any combination thereof; and a stabilizer.

In some embodiments, a liquid pharmaceutical composition disclosed herein comprises between about 10 to about 45% w/v, at least about 30% w/v, or between about 30 to about 45% w/v of the LD-Tyr, or an enantiomer, diastereomer, racemate, ion, zwitterion, pharmaceutically acceptable salt thereof, or any combination thereof.

In some embodiments, the stabilizer is present in an amount of about 0.1 to about 30% w/v.

In some embodiments, the stabilizer comprises a base. In some embodiments, the base is selected from the group consisting of arginine, NaOH, NH₄OH, tris(hydroxymethyl)aminomethane (TRIS), ethylendiamine, diethylamine, ethanolamine, diethanolamine, meglumine, and any combination thereof. In some embodiments, the base is selected from the group consisting of arginine, NH₄OH, ethylendiamine, diethylamine, ethanolamine, diethanolamine, meglumine, and any combination thereof. In some embodiments, the base is selected from the group consisting of L-Arg, diethylamine, and a combination thereof. In some embodiments, the base is selected from the group consisting of L-Arg, ethanolamine, and a combination thereof.

In some embodiments, the liquid pharmaceutical composition comprises between about 0.1 to about 30% w/v of the base. In some embodiments, the liquid pharmaceutical composition comprises between about 1.5 to about 20% w/v of the base.

In some embodiments, a liquid pharmaceutical composition disclosed herein has a pH in the range of between about 5 to about 10 at about 25° C. In some embodiments, a liquid pharmaceutical composition disclosed herein has a pH in the range of between about 8 to about 10. In some embodiments, a liquid pharmaceutical composition disclosed herein has a pH in the range of between about 8 to about 9.

In some embodiments, a liquid pharmaceutical composition disclosed herein can include a free base of the compound of formula II and a counterion.

In some embodiments, a liquid pharmaceutical composition disclosed herein can also include a decarboxylase inhibitor. For example, in some embodiments, the decarboxylase inhibitor is carbidopa. In some embodiments, a liquid pharmaceutical composition disclosed herein can include between about 0.25 to about 2.0% w/v of the decarboxylase inhibitor.

Any of the aforementioned liquid pharmaceutical compositions described herein can further include an antioxidant or a combination of two or more antioxidants. For example, in some embodiments, a liquid pharmaceutical composition described herein can include an antioxidant selected from the group consisting of ascorbic acid or a salt thereof, a cysteine, e.g., N-acetyl cysteine (NAC), a bisulfite or a salt thereof, glutathione, a tyrosinase inhibitor, a Cu²⁺ chelator, and any combination thereof. In some embodiments, a liquid pharmaceutical composition described herein can include between about 0.05 to about 1.5% w/v of an antioxidant or a combination of antioxidants. In some embodiments, the liquid pharmaceutical composition comprises a combination of ascorbic acid and NAC.

Any of the aforementioned liquid pharmaceutical composition described herein can further include at least one of: a catechol-O-methyltransferase (COMT) inhibitor, a monoamine oxidase (MAO) inhibitor, a surfactant, a buffer, an acid, a base, a solvent, or any combination thereof. In some embodiments, the liquid pharmaceutical composition comprises between about 5.0 to about 40.0% w/v of a buffer, base, or solvent. For example, in some embodiments, a liquid pharmaceutical composition described herein can include a solvent, wherein the solvent may be N-methylpyrrolidone (NMP), tris(hydroxymethyl)aminomethane (tromethamine, TRIS), an ether such as tetrahydrofuran and 1,4-dioxane an amide, such as N,N-dimethylformamide and N-methylpyrrolidone, a nitrile, such as acetonitrile, a halogenated aliphatic hydrocarbon, such as chloroform and dichloromethane, an aromatic hydrocarbon, such as toluene or any combination thereof. It is noted that certain materials, such as tromethamine (TRIS) may be added to the composition and function, e.g., as a base, buffer, solvent, or any combination thereof. In some embodiments, a liquid pharmaceutical composition described herein can include a surfactant, wherein the surfactant is Tween-80. In some embodiments, a liquid pharmaceutical composition described herein can include a solvent and a surfactant, where the solvent is NMP and the surfactant is Tween-80. In some embodiments, the liquid pharmaceutical composition can include between about 0.1 to about 1.0% w/v of the surfactant, for example, 0.1 to about 1.0% w/v of Tween-80. In some embodiments, the liquid pharmaceutical composition can include between about 5.0 to about 40.0% w/v of the solvent, for example, between about 5.0 to about 40.0% w/v of NMP.

In certain embodiments, the solvent is TRIS. In certain embodiments, the stabilizer includes polyethylene glycol.

In certain embodiments, the liquid pharmaceutical composition comprises less than about 1.5% w/v LD-Tyr-diketopiperazine after two weeks at 2-8° C. In certain embodiments, the liquid pharmaceutical composition comprises less than about 0.8% w/v LD-Tyr-diketopiperazine after two weeks at 2-8° C. In certain embodiments, the liquid pharmaceutical composition comprises less than about 5.0% w/v LD-Tyr-diketopiperazine after two weeks at 25° C. In certain embodiments, the liquid pharmaceutical composition comprises no more than about 4% w/v LD-Tyr-diketopiperazine after two weeks at 25° C.

Also disclosed herein is a method of treating a neurodegenerative condition and/or a condition characterized by reduced levels of dopamine in the brain, wherein the method comprises administering a liquid pharmaceutical composition as described herein.

For example, disclosed herein is a method of treating a neurodegenerative condition and/or a condition characterized by reduced levels of dopamine in the brain, wherein the method comprises administering a liquid pharmaceutical composition including LD-Tyr and a stabilizer.

Disclosed herein is a method of treating a neurodegenerative condition and/or a condition characterized by reduced levels of dopamine in the brain, wherein the neurodegenerative condition is Parkinson's disease.

In some embodiments of the disclosed methods of treating, the liquid pharmaceutical composition is administered concomitantly with an additional active ingredient. For example, in some embodiments, the additional active ingredient is a decarboxylase inhibitor, a COMT inhibitor, a MAO inhibitor, or any combination thereof.

In some embodiments of the methods of treating disclosed herein, the liquid pharmaceutical composition is administered substantially continuously. In some embodiments, the liquid pharmaceutical composition is administered subcutaneously.

Also disclosed herein is a liquid pharmaceutical composition for use in treating a neurodegenerative condition and/or a condition characterized by reduced levels of dopamine in the brain.

Disclosed herein is a liquid pharmaceutical composition for use in treating a neurodegenerative condition and/or a condition characterized by reduced levels of dopamine in the brain, wherein the neurodegenerative condition is Parkinson's disease.

According to some embodiments, the liquid pharmaceutical composition is administered concomitantly to the patient with an additional active ingredient, e.g., a decarboxylase inhibitor, a COMT inhibitor, a MAO inhibitor, and any combination thereof.

According to some embodiments, the liquid pharmaceutical composition is administered substantially continuously to the patient. According to further embodiments, the liquid pharmaceutical composition is administered subcutaneously.

Embodiments of the invention are further directed to a method of treating Parkinson's disease in a patient in need thereof, comprising subcutaneously administering to the patient an effective amount of the liquid pharmaceutical formulation, as disclosed herein. Further embodiments of the invention are directed to uses of the liquid pharmaceutical formulation as disclosed herein for treating neurodegenerative conditions and/or conditions characterized by reduced levels of dopamine in the brain, such as Parkinson's disease.

Also disclosed herein is a process for preparing a liquid pharmaceutical composition, wherein said process comprises providing a pharmaceutically acceptable salt of LD-Tyr; combining the pharmaceutically acceptable salt with at least one solvent thereby forming a solution, gel, cream, emulsion, or suspension; combining the solution, gel, cream, emulsion or suspension with a stabilizer; and adjusting the pH of the solution, gel, cream, emulsion, or suspension, to a physiologically acceptable pH value, thereby providing the liquid pharmaceutical composition.

In some embodiments, a process for preparing a liquid pharmaceutical composition described herein includes providing a pharmaceutically acceptable salt of a LD-Tyr, an enantiomer, diastereomer, racemate, ion, zwitterion, pharmaceutically acceptable salt thereof, or any combination thereof.

In some embodiments of a process described herein, the LD-Tyr compound of Formula (II) in a pharmaceutically acceptable salt form is mixed with at least one solvent and at least one stabilizer, thereby forming a solution. In some embodiments, the process includes a step of adjusting the pH that comprises adding a basic solution. For example, in some embodiments, the process includes a step of adjusting the pH that comprises adding a basic solution, and the basic solution comprises NaOH. In some embodiments the process does not include heating.

### BRIEF DESCRIPTION OF THE DRAWINGS

FIG. 1 is a graph depicting rate of LD-Tyr-diketopiperazine (LD-Tyr-DKP) formation over time at 2-8° C. in LD-Tyr formulations comprising the indicated bases.

FIG. 2 is a graph depicting rate of LD-Tyr-diketopiperazine (LD-Tyr-DKP) formation over time at 25° C. in LD-Tyr formulations comprising the indicated bases.

FIG. 3 presents the K_{RBC/PL} for LD-Tyr following incubation in rat blood.

FIG. 4 presents the K_{RBC/PL} for LD-Tyr following incubation in minipig blood.

FIG. 5 presents the K_{RBC/PL} for LD-Tyr following incubation in human blood.

FIG. 6 presents the composite mean plasma LD-Tyr concentration vs. time profiles in domestic pigs following continuous subcutaneous infusion for 18 hours.

FIG. 7 presents the composite mean plasma LD concentration vs. time profiles in domestic pigs following continuous subcutaneous infusion of LD-Tyr for 18 hours.

FIG. 8 presents the composite mean plasma LD concentration vs. time profiles in domestic pigs following continuous subcutaneous infusion of high concentrations of LD-Tyr and 1% CD for 18 hours.

FIG. 9 presents the composite mean plasma LD concentration vs. time profiles in domestic pigs following continuous subcutaneous infusion of high concentrations of LD-Tyr and 0.5% CD for 18 hours.

FIG. 10 presents the composite mean plasma LD concentration vs. time profiles in domestic pigs following continuous subcutaneous infusion of LD-Tyr and varying amounts of CD for 18 hours.

FIG. 11 presents the composite mean normalized LD plasma concentration-versus-time profiles in domestic pigs following an 18 hour continuous SC infusion of 30% LD-Tyr together with different CD concentrations.

FIG. 12 presents the composite mean plasma LD concentration-versus-time profiles following different volumes bolus infusions and 2-hr continuous SC infusion of 30% LD-Tyr and 1% CD in domestic pigs.

FIG. 13 shows the composite mean plasma LD-Tyr and LD concentration-versus-time profiles for the 2700 mg dosing regimen. PK parameters are also shown.

FIG. 14 shows the composite mean plasma LD-Tyr and LD concentration-versus-time profiles for the 5400 mg dosing regimen. PK parameters are also shown.

FIG. 15 shows the composite mean plasma LD concentration-versus-time profiles for the 2700 and 5400 mg dosing regimens.

### DETAILED DESCRIPTION OF THE INVENTION

The features and other details of the disclosure will now be more particularly described. Certain terms employed in the specification, examples, and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The terms “treat,” “treatment,” “treating,” and the like are used herein to generally refer to obtaining a desired pharmacological and/or physiological effect. The effect may be therapeutic in terms of partially or completely curing a disease and/or adverse effect attributed to the disease. The term “treatment” as used herein includes any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refer to any and all solvents, dispersion media, coatings, isotonic and absorption delaying agents, and the like, that are compatible with pharmaceutical administration.

The terms “pharmaceutical composition” and “pharmaceutical formulation” as used herein refer to a composition or formulation comprising at least one biologically active compound, for example, a levodopa amino acid conjugate, or a pharmaceutically acceptable salt thereof, as disclosed herein, formulated together with one or more pharmaceutically acceptable excipients. It is noted that the terms “formulation” and “composition” are interchangeable unless specifically mentioned otherwise or unless a person skilled in the art would have understood otherwise.

The term “pharmaceutically acceptable salt(s)” as used herein refers to salts of acidic or basic groups that may be formed with the conjugates used in the compositions disclosed herein.

“Individual,” “patient,” or “subject” are used interchangeably and include any animal, including mammals, mice, rats, other rodents, rabbits, dogs, cats, swine, cattle, sheep, horses, or non-human primates, and humans. In some embodiments, the mammal treated in the methods of the invention is a human suffering from neurodegenerative condition, such as Parkinson's disease and/or a disease or symptom caused by a decrease in dopamine concentration in the brain.

The term “about” as used herein, unless specifically mentioned otherwise, or unless a person skilled in the art would have understood otherwise, is considered to cover a range of ±10% of the listed value(s). It is further noted that any value provided may also be considered to cover a range of ±10% of that value, even without the use of the term “about”. This includes the values in the examples section, which may vary according to the utensils and machinery used, the purity of the compounds, etc.

The term “stable” as used herein, unless specifically mentioned otherwise, refers to a state of a substance that does not or is difficult to decompose in a solution. Therefore, the term “stable” as used herein means that, for example, when a solution of the substance is prepared and a peak area ratio of the substance measured using an area percentage method of high performance liquid chromatography (HPLC) immediately after the preparation of the solution is compared to a peak area ratio of the substance after being left at 25° C. for about 1 day, a decrease in peak area ratio is not observed or the degree of the decrease in peak area ratio is low. In another embodiment, the term “stable” refers to a substance that was physically stable after an indicated period of time, such that, upon visual view of the substance, e.g., formulation, under magnification of at least ×1.75, no precipitants were visible.

The terms “stabilizer” as used herein, unless specifically mentioned otherwise, or unless a person skilled in the art would have understood otherwise, refers to a substance that prevents or slows precipitation or the development of impurities in a substance, e.g., a liquid pharmaceutical formulation. Thus, the term “stabilizer” used herein refers to any excipient that provides the liquid pharmaceutical composition with enhanced stability, e.g., physical and/or chemical stability. The stabilizer may be, e.g., a solvent, buffer, base, acid, or any combination thereof and therefore, any excipient referred to herein, e.g., as a solvent, buffer, etc., may be considered a stabilizer as well.

The term “liquid” as used herein, unless specifically mentioned otherwise, or unless a person skilled in the art would have understood otherwise, refers to any type of fluid, including gels, aqueous and non-aqueous compositions, and the like.

The term “use in combination” as used herein, unless specifically mentioned otherwise, means administration of two or more active ingredients in combination, including administration of these active ingredients at the same time, either separately or in the same composition, and also includes administration of two or more active ingredients consecutively on the same day, and administration of the active ingredients separated from each other for a predetermined time period, and further includes administration of two or more active ingredients on different days.

The term “concomitant” as used herein, unless specifically mentioned otherwise, or unless a person skilled in the art would have understood otherwise, refers to any type of combined administration of two or more active ingredients, including administration of those active ingredients at the same time, either in separate or the same composition, as well as administering the two or more active ingredients sequentially, consecutively, on the same day, with a predefined period of time separating the administration of the active ingredients from one another, and the like.

The terms “continuously” and “substantially continuously” as used herein, unless specifically mentioned otherwise, or unless a person skilled in the art would have understood otherwise, refer to a period of time during which a composition is administered over the entire period of time, with intermissions of less than about 24 hours, about 12 hours, about five hours, about three hours, about one hour, about 30 minutes, about 15 minutes, about five minutes or about one minute. The period of time during which a composition is administered may be at least about six hours, about eight hours, about 12 hours, about 15 hours, about 18 hours, about 21 hours, about 24 hours, three days, seven days, two weeks, a month, three months, six months, a year, two years, three years, five years, ten years, etc.

The term “physiologically acceptable pH value” and the like, as used herein, unless specifically mentioned otherwise, or unless a person skilled in the art would have understood otherwise, refers to pH values in the range of between about 4.5 to about 10. It is further noted that when pH values are provided, including in the examples, the values may be in the range of about ±0.1 and/or ±10% of the listed value(s), such that if the measured pH is 8.1, the same formulation may be prepared to provide a pH of about 8.0 or 8.2. Such differences may be due to temperature changes, various measuring devices, etc.

### I. Prodrugs

The compound of the present invention or a pharmaceutically acceptable salt thereof has a good conversion efficiency to levodopa and is useful as a prophylactic or therapeutic agent for a neurodegenerative disease and/or a disease or symptom caused by a decrease in dopamine concentration in the brain, for example, Parkinson's disease.

Further, the compound of the present invention or a pharmaceutically acceptable salt thereof is highly soluble, and further, is highly stable in a solution and is easy to be handled in a solution state, and thus, is highly suitable for use in a form of a steady delivery system.

In the present invention, an alkyl refers to a linear or branched saturated hydrocarbon group having 1-6 carbon atoms (C₁₋₆). In particular, a group having 1-4 carbon atoms (C₁₋₄) is preferable. Specific examples thereof include methyl, ethyl, n-propyl, i-propyl, n-butyl, t-butyl, n-pentyl, i-pentyl, n-hexyl, and the like. In particular, methyl is preferable.

An alkanoyl refers to a monovalent group in which the above-described alkyl is bonded to a carbonyl, and examples thereof include a linear or branched alkyl-CO— having 1-6 carbon atoms (C₁₋₆). Specific examples thereof include acetyl, propionyl, butyryl, pivaloyl, pentanoyl, hexanoyl, heptanoyle, and the like. In particular, acetyl is preferable.

An amino acid side chain refers to an amino acid side chain of a natural, synthetic, non-natural or non-protein-producing amino acid, and examples of the amino acid include arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, lanthionine, selenocysteine, pyrrolidine, ADDA amino acid ((2S,3S,4E,6E,8S,9S)-3-amino-9-methoxy-2,6,8-trimethyl-10-phenyldeca-4,6-dienoic acid), 0-alanine, 4-aminobenzoic acid, γ-aminobutyric acid, S-aminoethyl-L-cysteine, 2-aminoisobutyric acid, aminolevulinic acid, azetidine-2-carboxylic acid, canalin, canavanine, carboxyglutamic acid, chloroalanine, cystine, dehydroalanine, diaminopimelic acid, dihydroxyphenylglycine, endouracidine, homoserine, 4-phenylglycine, hydroxyproline, hypusine, β-Leucine, norleucine, norvaline, ornithine, penicillamine, placohipaphorin, pyroglutamic acid, quisqualic acid, sarcosine, theanine, tranexamic acid, trichromic acid, 3,4-dihydroxyphenylalanine, and the like. In particular, glutamic acid, valine, alanine, lysine, 3,4-dihydroxyphenylalanine or tyrosine is preferable.

Here, the amino acid side chain in the present invention may be substituted, and examples thereof include —P(O)(OR₆)₂ (where R₆ is a hydrogen, an alkyl, or the like), a glucosyl group (such as [(2R,3S,4R,5S,6R)-3,4,5-trihydroxy-6-(hydroxymethyl)tetrahydropyran-2-yl]), a group that bonds to another adjacent group to form an alkylene group that may be substituted (examples of substituent groups include an alkyl group, an alkoxy group, and the like), and the like. Examples of substituted amino acid side chains include those in which a tyrosine side chain, a serine side chain, a threonine side chain, or an amino acid side chain having a hydroxy such as (3,4-dihydroxyphenyl)methyl is substituted, and specific examples thereof include phosphonooxymethyl, (4-phosphonooxyphenyl)methyl, [4-[(2S,3R,4S,5S,6R)-3,4,5-trihydroxy-6-(hydroxymethyl)tetrahydropyran-2-yl]oxyphenyl]methyl, (2,2-dimethyl-1,3-benzodioxol-5-yl)methyl, (2-ethoxy-2-methyl-1,3-benzodioxol-5-yl)methyl, and the like.

In the present invention, monosaccharides that are the source of glycosyl include aldoses such as glucose (dextrose), ribose, erythrose, xylose, arabinose, mannose, and galactose, as well as ketoses such as ribulose, psicose, fructose, sorbose, and tagatose. These monosaccharides may be of d-form, 1-form or dl-form. Here, a monosaccharide in the present invention may be substituted, and examples of substituent groups include a carbonyl group, an acetylamino group, a sulfinooxy group, a phosphonooxy group, and the like. Specific examples of substituted monosaccharides include glucuronic acid, N-acetylglucosamine, Glucopyranoside-6-(hydrogen sulfate), and the like.

The present invention includes as an embodiment a levodopa amino acid complex represented by the general formula (I) or (III) or a pharmaceutically acceptable salt thereof:

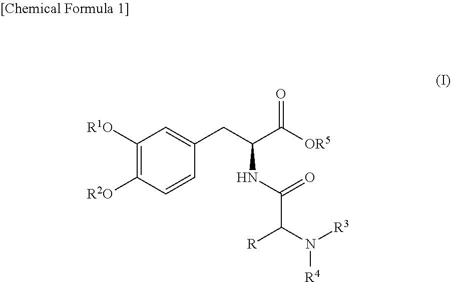

wherein R is an amino acid side chain that may be substituted;
R¹ and R² may be the same or different, and are each independently a hydrogen atom, a C₁-C₆ alkyl, a C₁-C₆ alkanoyl, a phosphono, a sulfino, or a glycosyl that may be substituted, provided that R¹ and R² are not hydrogen atom at the same time;
R³ and R⁴ may be the same or different, and are each independently a hydrogen atom or a C₁-C₆ alkyl; and
R⁵ is a hydrogen atom, or

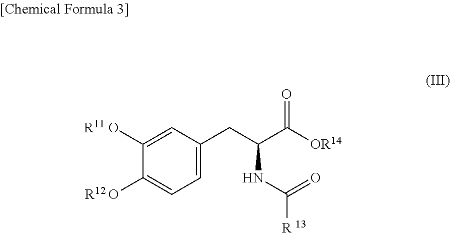

wherein R¹¹ and R¹² are the same or different and are each a hydrogen, an alkyl, an alkanoyl, a P(═O)(OH)₂, S(═O)(OH) or a glycosyl that may be substituted;
R¹³ is an alkyl that may be substituted, —R¹⁵—O—R¹⁶ or a 5-membered heterocyclyl containing at least one nitrogen atom, wherein R¹⁵ is an alkylene, and R¹⁶ is a hydrogen, an alkyl, P(═O)(OH)₂, S(═O)(OH), or a glycosyl that may be substituted; and
R¹⁴ is a hydrogen or an alkyl, provided that following compounds are excluded;
(2S)-2-[(2-aminoacetyl)amino]-3-(3,4-diacetyloxyphenyl)propanoic acid,
(2S)-2-[[(2S)-2-amino-6-[(2-chlorophenyl)methoxycarbonylamino]hexanoyl]amino]-3-(3,4-dimethoxyphenyl)propanoic acid,
(2S)-2-[[(2S)-2-amino-3-(3,4-dihydroxyphenyl)propanoyl]amino]-3-(4-hydroxy-3-methoxyphenyl)propanoic acid,
(2S)-2-[[(2S)-2-amino-3-phenylpropanoyl]amino]-3-(3,4-dimethoxyphenyl)propanoic acid,
(2S)-2-[[(2R)-2-amino-3-phenylpropanoyl]amino]-3-(3,4-diacetyloxyphenyl)propanoic acid, and
(2S)-2-[[(2S)-2-amino-5-methoxy-5-oxopentanoyl]amino]-3-(3,4-dimethoxyphenyl)propanoic acid.

When the compound (I) or (III) of the present invention has asymmetric carbon atoms in molecules thereof, stereoisomers (optical isomers, and diastereomeric isomers) based on the asymmetric carbon atoms can exist. The compound (I) or (III) of the present invention includes any one of these stereoisomers and mixtures thereof.

The compound (I) or (III) of the present invention includes compounds labeled with isotopes (for example, 3H, 13C, 14C, 15N, 18F, 32P, 35S, 125I, and the like) and include deuterium converters.

The compound (I) or (III) of the present invention can be used for pharmaceutical purposes either in a free form or in a form of a pharmaceutically acceptable salt or a form of a co-crystal. Examples of the pharmacologically acceptable salt of the compound (I) or (III) of the present invention include inorganic acid addition salts (such as salts of hydrochloric acid, hydrobromic acid, hydroiodic acid, sulfuric acid, phosphoric acid, and the like), organic acid addition salts (such as salts of methanesulfonic acid, benzenesulfonic acid, p-toluenesulfonic acid, formic acid, acetic acid, trifluoroacetic acid, oxalic acid, citric acid, malonic acid, fumaric acid, glutaric acid, adipic acid, maleic acid, tartrate acid, succinic acid, mandelic acid, malic acid, pantothenic acid, methyl sulfuric acid, and the like), inorganic base addition salts (such as salts of sodium, potassium, calcium, magnesium, and the like), salts of amino acids (such as salts of glutamic acid, aspartic acid, arginine, lysine, and the like), and the like.

The compound (I) or (III) of the present invention or a pharmacologically acceptable salt thereof includes any of intramolecular salts or adducts thereof, solvates or hydrates thereof, and the like.

The compound (I) or (III) of the present invention or a pharmaceutically acceptable salt thereof can be orally or parenterally administered alone or as a pharmaceutical composition that contains the compound (I) or (III) or a pharmaceutically acceptable salt thereof and a pharmacologically acceptable carrier. The pharmacologically acceptable carrier may be a carrier commonly used in the art, and examples thereof include diluents, binders (such as syrup, gum arabic, gelatin, sorbitol, tragacanth, and polyvinylpyrrolidone), excipients (such as lactose, sucrose, cornstarch, potassium phosphate, sorbitol, and glycine), lubricants (such as magnesium stearate, talc, polyethylene glycol, and silica), disintegrants (such as potassium starch), wetting agents (such as sodium lauryl sulfate), and the like. Further, when a liquid pharmaceutical composition such as an injection or an infusion is produced, a carrier commonly used in the art may be used, and examples thereof include aqueous solvents (such as water for injection, and purified water), isotonizing agents (such as sodium chloride, potassium chloride, glycerin, mannitol, sorbitol, boric acid, borax, glucose, and propylene glycol), buffers (such as a phosphoric acid buffer, an acetic acid buffer, a boric acid buffer, a carbonic acid buffer, a citric acid buffer, a tris buffer, a glutamic acid buffer, and an epsilon aminocaproic acid buffer), preservatives (such as methyl paraoxybenzoate, ethyl paraoxybenzoate, propyl paraoxybenzoate, chlorobutanol, benzyl alcohol, benzalconium chloride, sodium dehydroacetate, sodium edetate, boric acid, and borax), soothing agents (such as lidocaine hydrochloride, procaine hydrochloride, benzyl alcohol, and chlorobutanol), viscous agents (such as hydroxyethyl cellulose, hydroxypropyl cellulose, polyvinyl alcohol, and polyethylene glycol), stabilizers (such as sodium bisulfite, sodium thiosulfate, sodium edetate, sodium citrate, ascorbic acid, and dibutylhydroxytoluene), pH adjusting agents (such as hydrochloric acid, sodium hydroxide, phosphoric acid, and acetic acid), and the like. The liquid pharmaceutical composition can be produced by dissolving or dispersing the compound of the present invention described above in a solution to which these carriers are appropriately added. Such pharmaceutically acceptable additives can be appropriately selected by a person skilled in the art according to a purpose, and a condition such as an additive amount can also be appropriately set. Further, when necessary, a solubilizing agent or the like may be used.

A dosage form of such a pharmaceutical composition is not particularly limited, and examples thereof include conventional pharmaceutical formulations such as tablets, granules, capsules, powders, injections, inhalants, and suppositories. In particular, liquid pharmaceutical compositions containing injections can be used.

The compound (I) or (III) of the present invention or a pharmaceutically acceptable salt thereof can be formulated in a form of a liquid pharmaceutical composition, for example, as a formulation applicable to all appropriate routes of administration by parenteral administration such as bolus administration, continuous administration or sustained administration. Specifically, a liquid pharmaceutical composition containing the compound of the present invention can be formulated for subcutaneous administration, transdermal administration, intradermal administration, transmucosal administration, intravenous administration, intraarterial administration, intramuscular administration, intraperitoneal administration, intratracheal administration, intrathecal administration, intraduodenal administration, intrapleural administration, intranasal administration, sublingual administration, buccal administration, buccal administration, intestinal administration, intraduodenal administration, rectal administration, intraocular administration or oral administration. Further, the composition can also be formulated for inhalation or direct absorption through mucosal tissues.

A dosage amount of the compound (I) or (III) of the present invention or a pharmaceutically acceptable salt thereof varies depending on an administration method, and age, body weight, condition and the like of a patient. However, when administered as a liquid pharmaceutical composition, it is usually 1-200 mg/kg per day.

The compound (I) or (III) of the present invention or a pharmaceutically acceptable salt thereof can be used alone or in combination with one or more other therapeutic agents, depending on the disease to be treated (for example, Parkinson's disease). As such therapeutic agents, for example, one or more agents selected from a group consisting of dopamine decarboxylase inhibitors (such as carbidopa and benserazide), catechol-O-methyltransferase (“COMT”) inhibitors (such as entacapone and tolcapone), and monoamine oxidase A (“MAO-A”) or monoamine oxidase B (“MAO-B”) inhibitors (such as moclobemide, rasagiline, selegiline and safinamide) can be used.

The compound (I) or (III) of the present invention or a pharmaceutically acceptable salt thereof can be administered at the same time as the above-described therapeutic agents that can be used in combination, or can be administered separately. Further, when the compound of the present invention is used therapeutically together with the above-described therapeutic agents that can be used in combination, the compound of the present invention and the therapeutic agents can be administered in the same dosage form such as parenteral administration, and may be administered in different dosage forms such as parenteral administration for one and oral administration for the other.

The preferred embodiments of the present invention are shown in the following table.

TABLE 1

| Example | Japanese IUPAC name |
|---|---|
| 1 | (2S)-2-[[(2S)-2-aminopropanoyl]amino]-3-(3-hydroxy-4- |
| | phosphonooxyphenyl) propanoic acid |
| 2 | (2S)-2-[[(2S)-2-amino-3-(3,4-dihydroxyphenyl) |
| | propanoyl]amino]-3-(3-hydroxy-4-phosphonooxyphenyl) |
| | propanoic acid |
| 8 | (2S)-2-[[(2S)-2,6-diaminohexanoyl]amino]-3-(3-hydroxy-4- |
| | phosphonooxyphenyl) propanoic acid |
| 10 | (2S)-2-[[(2S)-2-amino-3-(4-phosphonooxyphenyl) |
| | propanoyl]amino]-3-(3,4-dihydroxyphenyl) propanoic acid |

The compound of the present invention or a pharmaceutically acceptable salt thereof can be produced, for example, as follows.

### General Synthesis Method (A)

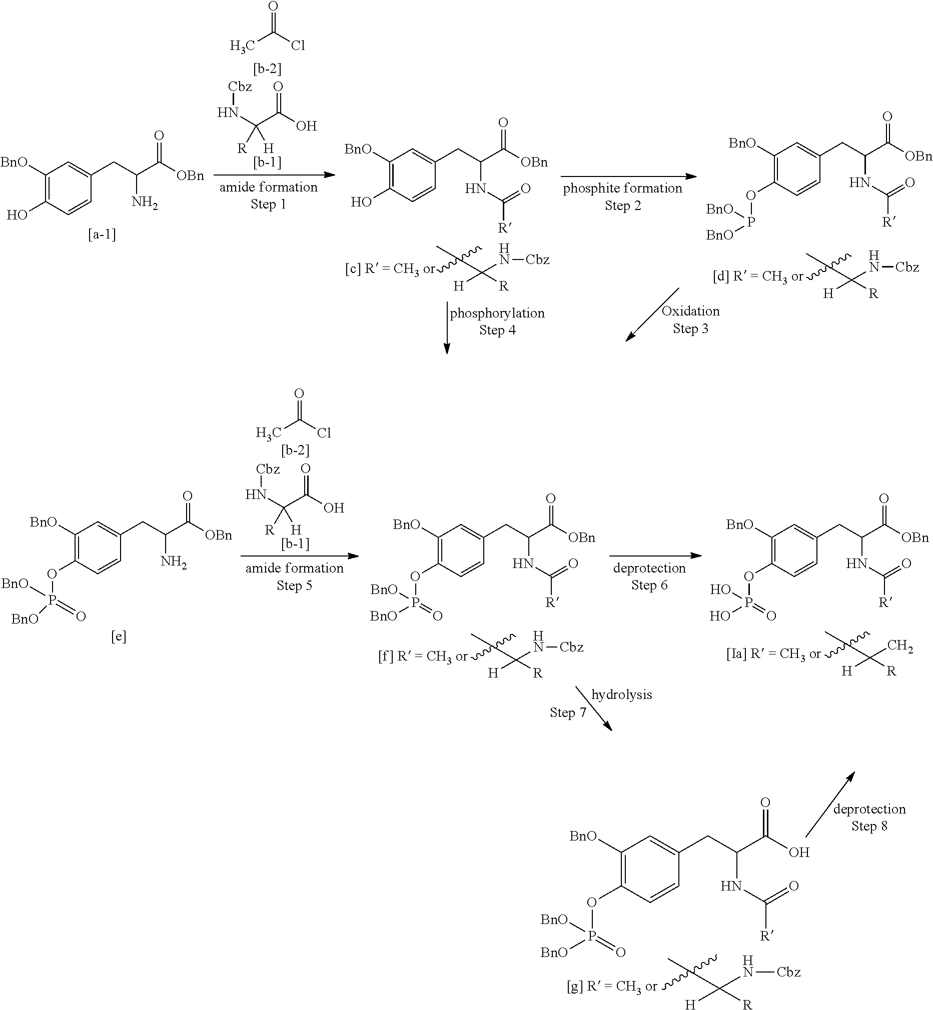

[wherein, Bn indicates a benzyl, Cbz indicates a carbobenzoxy, and the other symbols have the same meaning as above]

Among the target compounds [I] of the present invention, the compound represented by the general formula [Ia] can be produced, for example, as follows. The compound [a-1] and the compound [b-1] or the compound [b-2] are subjected to a condensation reaction to obtain the compound [c], and then, the compound [c] is subjected to phosphite esterification and oxidation, or is subjected to phosphate esterification, and thereby, the compound [f] is obtained. On the other hand, the compound [f] can also be obtained by condensing the compound [e] and the compound [b-1] or the compound [b-2]. The compound [Ia] can be produced by subjecting the compound [f] thus obtained to deprotection or to hydrolysis and then deprotection.

### Step 1

The condensation between the compound [a-1] and the compound [b-1] or a salt thereof or [b-2] can be carried out according to a conventional method in a suitable solvent in the presence or absence of a base, in the presence or absence of a condensing agent, and in the presence or absence of an activating agent. As the solvent, any solvent that does not affect the present reaction may be used. Examples of the solvent include: ethers such as tetrahydrofuran and 1,4-dioxane; amides such as N,N-dimethylformamide, N,N-dimethylacetamide and N-methylpyrrolidone; nitriles such as acetonitrile; halogenated aliphatic hydrocarbons such as chloroform and dichloromethane; aromatic hydrocarbons such as toluene; or a mixture of these compounds.

Examples of the base include triethylamine, diisopropylethylamine, 1,8-diazabicyclo[5.4.0]undec-7-ene, and the like. Examples of the condensing agent include O-(7-azabenzotriazol-1-yl)-N,N,N′,N′-tetramethyluronium hexafluorophosphate (HATU), 1-ethyl-3-(3-dimethylaminopropyl)carbodiimide hydrochloride, 1-(3-dimethylaminopropyl)-3-ethylcarbodiimide hydrochloride, and the like. Examples of the activating agent include 1-hydroxy-7-azabenzotriazole (HOAt), 1-hydroxybenzotriazole (HOBt), 4-dimethylaminopyridine and the like.

An amount of the compound [b-1] or [b-2] to be used can be 1.0-5.0 equivalents, preferably 1.0-2.0 equivalents, in molar ratio with respect to the compound [a-1].

An amount of the base to be used can be 1.0-5.0 equivalents, preferably 1.0-2.0 equivalents, in molar ratio with respect to the compound [a-1].

An amount of the condensing agent to be used can be 1.0-5.0 equivalents, preferably 1.0-2.5 equivalents, in molar ratio with respect to the compound [a-1].

An amount of the activating agent to be used can be 1.0-5.0 equivalents, preferably 1.0-2.5 equivalents, in molar ratio with respect to the compound [a-1].

The present reaction can be carried out at room temperature—under heating, for example, at room temperature—80° C., preferably at room temperature—50° C.

### Step 2

The condensation of the compound [c] and a phosphite esterifying agent can be carried out according to a conventional method in a suitable solvent in the presence of an activating agent. As the solvent, any solvent that does not affect the present reaction may be used. Examples of the solvent include: nitriles such as acetonitrile; halogenated aliphatic hydrocarbons such as chloroform and dichloromethane; or a mixture of these compounds. An example of the phosphite esterifying agent is dibenzyl N,N-diisopropyl phosphoramidite. An example of the activating agent is 1-tetrazole.

An amount of the phosphite esterifying agent to be used can be 1.0-5.0 equivalents, preferably 1.5-3.0 equivalents, in molar ratio with respect to the compound [c].

An amount of the activating agent to be used can be 1.0-5.0 equivalents, preferably 1.5-3.0 equivalents, in molar ratio with respect to the compound [c].

The present reaction can be carried out under ice-cooling—under heating, for example, at 0° C.-80° C., preferably at room temperature—50° C.

### Step 3

The oxidation of the compound [d] can be carried out according to a conventional method in a suitable solvent in the presence of an oxidizing agent. As the solvent, any solvent that does not affect the present reaction may be used. Examples of the solvent include: nitriles such as acetonitrile; halogenated aliphatic hydrocarbons such as chloroform and dichloromethane; or a mixture of these compounds. Examples of the oxidizing agent include a hydrogen peroxide solution, tert-butyl hydroperoxide, metachloroperbenzoic acid, and the like.

An amount of the oxidizing agent to be used can be 1.0-5.0 equivalents, preferably 1.5-3.0 equivalents, in molar ratio with respect to the compound [d].

The present reaction can be carried out under ice-cooling—under heating, for example, at 0° C.-80° C., preferably at room temperature—50° C.

### Step 4

The condensation of the compound [c] and a phosphate esterifying agent can be carried out according to a conventional method in a suitable solvent in the presence or absence of a base. As the solvent, any solvent that does not affect the present reaction may be used. Examples of the solvent include: nitriles such as acetonitrile; halogenated aliphatic hydrocarbons such as chloroform and dichloromethane; or a mixture of these compounds. Examples of the phosphate esterifying agent include dibenzylphosphoryl chloride, tetrabenzyl pyrophosphate, and the like. Examples of the base include: alkali metal alkoxides such as sodium t-butoxide and potassium t-butoxide; alkylamines such as triethylamine, diisopropylethylamine and 1,8-diazabicyclo[5.4.0]undec-7-ene; and the like.

An amount of the phosphate esterifying agent to be used can be 1.0-5.0 equivalents, preferably 1.5-3.0 equivalents, in molar ratio with respect to the compound [c].

An amount of the base to be used can be 1.0-5.0 equivalents, preferably 1.5-3.0 equivalents, in molar ratio with respect to the compound [c].

The present reaction can be carried out at room temperature—under heating, for example, at room temperature—100° C., preferably at room temperature—70° C.

### Step 5

The condensation of the compound [e] and the compound [b-1] or a salt thereof or [b-2] can be carried out in the same manner as the condensation of the compound [a-1] and the compound [b-1] or a salt thereof or [b-2] in the general synthesis method (A).

### Step 6

The deprotection of the compound [f] can be carried out according to a conventional method by a treatment with a catalyst in a suitable solvent in a hydrogen atmosphere.

As the solvent, any solvent that does not affect the present reaction may be used. Examples of the solvent include: ethers such as tetrahydrofuran and 1,4-dioxane; alcohols such as methanol, ethanol and isopropanol; water; or a mixture of these compounds; and the like.

Examples of the catalyst include palladium/carbon, and the like.

The present reaction can be carried out at room temperature—under heating, for example, at room temperature—80° C., preferably at room temperature—50° C.

### Step 7

The hydrolysis of the compound [g] can be carried out according to a conventional method in a suitable solvent in the presence of a base and water. As the solvent, any solvent that does not affect the present reaction may be used. Examples of the solvent include: alcohols such as methanol, ethanol and isopropanol; ethers such as tetrahydrofuran, 1,4-dioxane and 1,2-dimethoxyethane; water; or a mixture of these compounds; and the like. Examples of the base include: alkali metal hydroxides such as sodium hydroxide and lithium hydroxide; and the like.

The present reaction can be carried out under ice-cooling—under heating, for example, at 0° C.-50° C., preferably at a room temperature.

An amount of the base to be used can be 1.0-10.0 equivalents, preferably 1.0-4.0 equivalents, with respect to the compound [g].

### Step 8

The deprotection of the compound [g] can be carried out in the same manner as the deprotection of the compound [f] in the general synthesis method (A).

### General Synthesis Method (B)

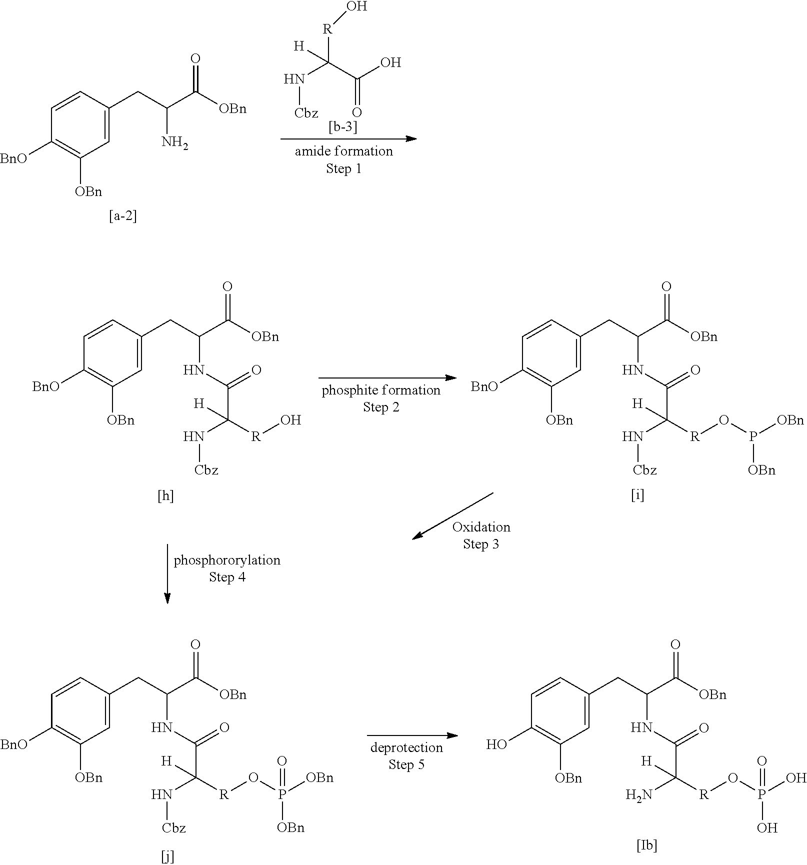

[wherein the symbols have the same meaning as above]

Among the target compounds [I] of the present invention, the compound represented by the general formula [Ib] can be produced, for example, as follows. The compound [a-2] and the compound [b-3] are subjected to a condensation reaction to obtain the compound [h], and then, the compound [h] is subjected to phosphite esterification and oxidation, or is subjected to phosphate esterification, and thereby, the compound [j] is obtained. The compound [Ib] can be produced by deprotecting the compound [j].

### Step 1

The condensation of the compound [a-2] and the compound [b-3] or a salt thereof can be carried out in the same manner as the condensation of the compound [a-1] and the compound [b-1] or a salt thereof in the general synthesis method (A).

### Step 2

The condensation of the compound [h] and a phosphite esterifying agent can be carried out in the same manner as the condensation of the compound [c] and a phosphite esterifying agent in the general synthesis method (A).

### Step 3

The oxidation of the compound [i] can be carried out in the same manner as the oxidation of the compound [d] in the general synthesis method (A).

### Step 4

The condensation of the compound [h] and a phosphate esterifying agent can be carried out in the same manner as the condensation of the compound [c] and a phosphate esterifying agent in the general synthesis method (A).

### Step 5

The deprotection of the compound [j] can be carried out in the same manner as the deprotection of the compound [f] in the general synthesis method (A).

### General Synthesis Method (C)

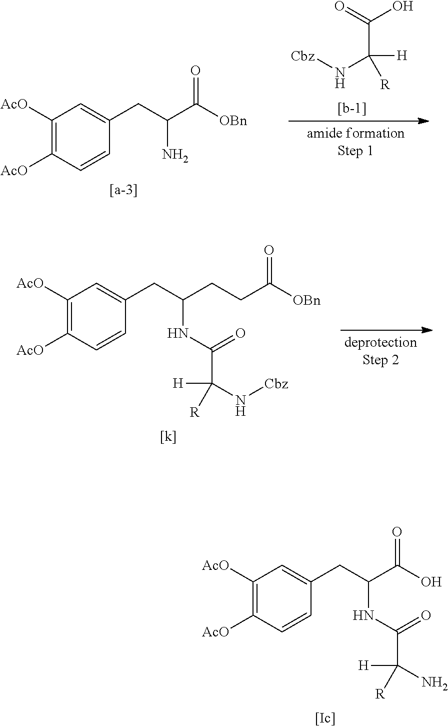

[wherein Ac indicates an acetyl, and the other symbols have the same meaning as above]

Among the target compounds [I] of the present invention, the compound represented by the general formula [Ic] can be produced, for example, as follows. The compound [a-3] and the compound [b-1] are subjected to a condensation reaction to obtain the compound [k], and then, the compound [k] is subjected to deprotection, and thereby, the compound [Ic] can be produced.

### Step 1

The condensation of the compound [a-3] and the compound [b-1] or a salt thereof can be carried out in the same manner as the condensation of the compound [a-1] and the compound [b-1] or a salt thereof in the general synthesis method (A).

### Step 2

The deprotection of the compound [k] can be carried out in the same manner as the deprotection of the compound [f] in the general synthesis method (A).

### General Synthesis Method (D)

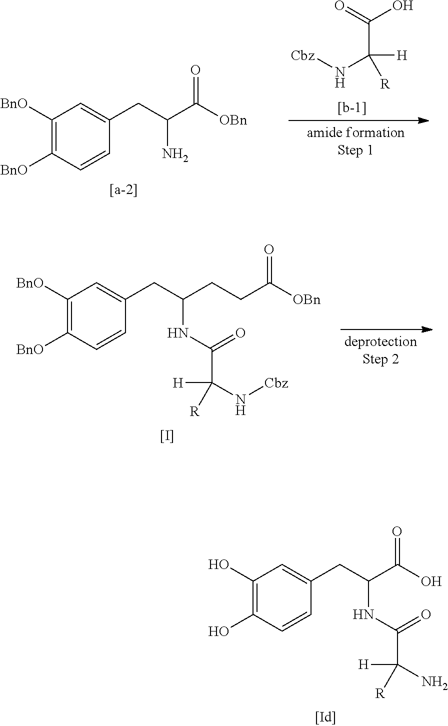

[wherein the symbols have the same meaning as above]

Among the target compounds [I] of the present invention, the compound represented by the general formula [Id] can be produced, for example, as follows. The compound [a-2] and the compound [b-1] are subjected to a condensation reaction to obtain the compound [1], and then, the compound [1] is subjected to deprotection, and thereby, the compound [Id] can be produced.

### Step 1

The condensation of the compound [a-2] and the compound [b-1] or a salt thereof can be carried out in the same manner as the condensation of the compound [a-1] and the compound [b-1] or a salt thereof in the general synthesis method (A).

### Step 2

The deprotection of the compound [1] can be carried out in the same manner as the deprotection of the compound [f] in the general synthesis method (A).

The starting compounds in the above methods can be produced in the same manner as in known methods and/or in methods described in Examples described later.

Introduction of a protecting group into a functional group and removal of a protecting group of a functional group can be carried out with reference to known methods (PROTECTIVE GROUPS in ORGANIC SYNTHESIS (by Theodora W. Greene, Peter G. M. Wuts), and the like).

A compound of the present invention or a starting compound thereof produced as described above is isolated and purified in a free form thereof or as a salt thereof. A salt can be produced by a commonly used salt preparation process. Isolation and purification can be carried out by applying conventional chemical procedures such as extraction, concentration, crystallization, filtration, recrystallization, and various types of chromatography.

When the compound of the present invention or a pharmaceutically acceptable salt thereof exists as optical isomers based on asymmetric carbon atoms, it can be separated into individual optical isomers by ordinary optical resolution means (for example, a fractional crystallization method, and a separation method using a chiral column). Further, an optical isomer can also be synthesized using an optically pure starting material. Further, an optical isomer can also be synthesized by stereoselectively performing each reaction using an asymmetric auxiliary group or an asymmetric catalyst.

The compound of the present invention or a pharmaceutically acceptable salt thereof is useful for prevention or treatment of a neurodegenerative disease and/or a disease or symptom caused by a decrease in dopamine concentration in the brain. Specifically, the compounds and the combination medicament of the present invention are useful for prevention or treatment of Parkinson's disease, secondary parkinsonism, Huntington's disease, Parkinson's disease-like syndrome, progressive supranuclear palsy (PSP), multiple system atrophy (MSA), amyotrophic lateral sclerosis (ALS), Shy-Drager syndrome, dystonia, Alzheimer's disease, Lewy body dementias (LBD), akinesia, bradykinesia, and hypokinesia, and are preferably useful for prevention or treatment of Parkinson's disease. Further, the compound and a combination medicament of the present invention are useful for prevention or treatment of diseases or symptoms caused by a brain damage including carbon monoxide poisoning or manganese poisoning, or diseases or symptoms associated with neurological diseases or neurological disorders including alcoholism, drug addiction or erectile dysfunction.

### II. Stable LD-Tyr Formulations

Embodiments of the invention are directed to a liquid pharmaceutical composition comprising a levodopa-tyrosine conjugate of the formula (II):

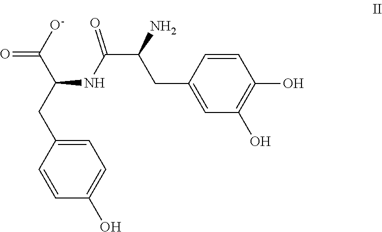 [SMILES: N[C@@H](Cc1ccc(O)c(O)c1)C(=O)N[C@@H](Cc1ccc(O)cc1)C(=O)[O-]]

an enantiomer, diastereomer, racemate, ion, zwitterion, pharmaceutically acceptable salt thereof, or any combination thereof, and a stabilizer.

According to some embodiments, the liquid pharmaceutical composition comprises an LD-Tyr compound in a pharmaceutically acceptable salt form. According to some embodiments, the LD-Tyr salt is selected from a trifluoroacetic acid (TFA) salt, an HCl salt, fumaric acid salt, lactate salt, maleic acid salt, gluceptic acid salt, phosphoric acid salt, sulfuric acid salt, HBr salt, nitric acid salt, acetic acid salt, propionic acid salt, hexanoic acid salt, cyclopentanepropionic acid salt, glycolic acid salt, pyruvic acid salt, lactic acid salt, hippuric acid salt, methanesulfonic acid salt, ascorbic acid salt, malonic acid salt, oxalic acid salt, maleic acid salt, tartaric acid salt, citric acid salt, succinic acid salt, benzoic acid salt, cinnamic acid salt, a sulfonic acid salt, lauryl sulfuric acid salt, gluconic acid salt, glutamic acid salt, hydroxynaphthoic acid salt, salicylic acid salt, stearic acid salt, muconic acid salt, an alkali metal salt, such as lithium salt, sodium salt or potassium salt, an alkaline earth metal salt, such as calcium salt or magnesium salt, an aluminum salt, an ethanolamine salt, diethanolamine salt, triethanolamine salt, N-methylglucamine salt, dicyclohexylamine salt, adipate salt, alginate salt, ascorbate salt, aspartate salt, benzenesulfonate salt, bisulfate salt, borate salt, butyrate salt, camphorate butyrate salt, camphorsulfonate butyrate salt, digluconate butyrate salt, dodecylsulfate butyrate salt, ethanesulfonate butyrate salt, glucoheptonate butyrate salt, glycerophosphate butyrate salt, gluconate butyrate salt, hemisulfate butyrate salt, heptanoate butyrate salt, hydroiodide butyrate salt, 2-hydroxy-ethanesulfonate butyrate salt, lactobionate butyrate salt, laurate butyrate salt, methanesulfonate butyrate salt, 2-naphthalenesulfonate butyrate salt, nicotinate butyrate salt, oleate butyrate salt, palmitate butyrate salt, pamoate butyrate salt, pectinate butyrate salt, persulfate butyrate salt, 3-phenylpropionate butyrate salt, phosphate butyrate salt, picrate butyrate salt, pivalate butyrate salt, tartrate butyrate salt, thiocyanate butyrate salt, p-toluenesulfonate butyrate salt, undecanoate butyrate salt, valerate salts, or any combination thereof.

The liquid pharmaceutical composition of the invention may comprise between about 2.5 to about 70% w/v of an LD-Tyr compound, an enantiomer, diastereomer, racemate, ion, zwitterion, pharmaceutically acceptable salt thereof, or any combination thereof, or any combination of two or more LD-Tyr enantiomers, diastereomers, racemates, ions, zwitterions, pharmaceutically acceptable salts thereof, or any combination thereof. According to some embodiments, the liquid pharmaceutical composition comprises between about 2.5 to about 5% w/v, between about 5 to about 10% w/v, between about 10 to about 15% w/v, between about 15 to about 20% w/v, between about 20 to about 25% w/v, between about 25 to about 30% w/v, at least about 30%, between about 30 to about 35% w/v, between about 35 to about 40% w/v, between about 30 to about 45% w/v, between about 30 to about 50% w/v, between about 30 to about 55% w/v, between about 30 to about 60% w/v, between about 30 to about 65% w/v, between about 30 to about 70% w/v, between about 40 to about 45% w/v, between about 45 to about 50% w/v, between about 50 to about 55% w/v, between about 55 to about 60% w/v, between about 60 to about 65% w/v, between about 65 to about 70% w/v, between about 10 to about 12.5% w/v, between about 12.5 to about 17.5% w/v, between about 17.5 to about 22.5% w/v, between about 22.5 to about 27.5% w/v, between about 27.5 to about 32.5% w/v, between about 32.5 to about 37.5% w/v, between about 37.5 to about 42.5% w/v, between about 42.5 to about 45% w/v, about 10% w/v, about 12.5% w/v, about 15% w/v, about 17.5% w/v, about 20% w/v, about 22.5% w/v, about 25% w/v, about 27.5% w/v, about 30% w/v, about 32.5% w/v, about 35% w/v, about 37.5% w/v, about 40% w/v, about 42.5% w/v, about 45% w/v, about 47.5% w/v, about 50% w/v, about 52.5% w/v, about 55% w/v, about 57.5% w/v, about 60% w/v, about 62.5% w/v, about 65% w/v, about 67.5% w/v, about 70% w/v of an LD-Tyr compound, an enantiomer, diastereomer, racemate, ion, zwitterion, pharmaceutically acceptable salt thereof, or any combination thereof, or any combination of two or more LD-Tyr enantiomers, diastereomers, racemates, ions, zwitterions, pharmaceutically acceptable salts thereof, or any combination thereof.

A liquid formulation as described herein comprises a stabilizer that prevents or slows precipitation of the formulation and/or that prevents or slows the accumulation of impurities in the formulation. LD-Tyr has a propensity to form a diketopiperazine (DKP) impurity as shown in the scheme below.

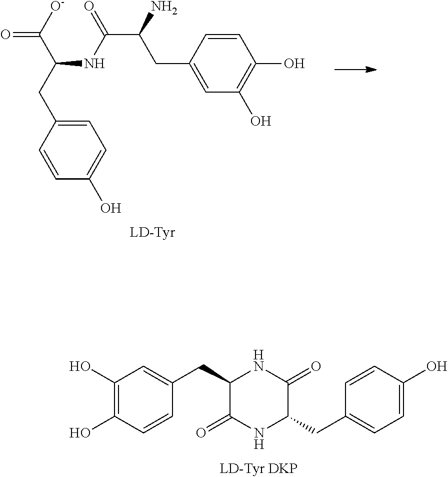 [SMILES: N[C@@H](Cc1ccc(O)c(O)c1)C(=O)N[C@@H](Cc1ccc(O)cc1)C(=O)[O-] ; O=C1N[C@H](Cc2ccc(O)c(O)c2)C(=O)N[C@H]1Cc1ccc(O)cc1]

Accordingly, in certain embodiments, a stabilizing agent as described herein is added to or is included in a liquid pharmaceutical composition and prevents diketopiperazine formation. Thus, in certain embodiments, the liquid pharmaceutical composition comprises less than about 1.5% w/v LD-Tyr-diketopiperazine (LD-Tyr DKP) after two weeks at 2-8° C. In certain embodiments, the liquid pharmaceutical composition comprises less than about 0.8% w/v LD-Tyr-diketopiperazine after two weeks at 2-8° C. For example, the liquid pharmaceutical composition can comprise less than about 0.8% w/v, 0.7% w/v, 0.6% w/v, 0.5% w/v, 0.4% w/v, 0.3% w/v, 0.2% w/v or 0.1% w/v LD-Tyr-diketopiperazine after two weeks at 2-8° C. In certain embodiments, the liquid pharmaceutical composition comprises less than about 4.0% w/v LD-Tyr-diketopiperazine after two weeks at 25° C. For example, the liquid pharmaceutical composition can comprise less than about 5.0% w/v, 4.5% w/v, 4.0% w/v, 3.5% w/v, 3.0% w/v, 2.5% w/v, 2.0% w/v, 1.5% w/v or 1.0% w/v LD-Tyr-diketopiperazine after two weeks at 25° C. In certain embodiments, the liquid pharmaceutical composition comprises no more than about 4.0% w/v LD-Tyr-diketopiperazine after two weeks at 25° C. Methods for measuring DKP are known in the art and include, for example, high-performance liquid chromatography (HPLC) and liquid chromatography-mass spectrometry (LC-MS).

In certain embodiments, the liquid pharmaceutical composition comprises between about 0.1% to about 30% w/v of the stabilizer. In certain embodiments, the liquid pharmaceutical composition comprises between about 1.5 to about 20% w/v of the stabilizer. For example, the liquid pharmaceutical composition can comprise between about 1.5 and about 5.0%, about 1.5 and about 10.0% w/v, about 1.5 and about 15.0% w/v, about 1.5 and about 20.0% w/v, about 1.5 and about 25.0% w/v, about 3.0 and about 5.0% w/v, about 3.0 and about 10.0% w/v, about 3.0 and about 15.0% w/v, about 3.0 and about 20.0% w/v, about 3 and about 25.0% w/v, about 3.0 and about 30.0% w/v, about 5.0 and about 10.0% w/v, about 5 and about 15.0% w/v, about 5.0 and about 20.0% w/v, about 5.0 and about 25.0% w/v, about 5.0 and about 30.0% w/v, about 7.0 and about 10.0% w/v, about 7.0 and about 15.0% w/v, about 7.0 and about 20.0% w/v, about 7.0 and about 25.0% w/v, about 7.0 and about 30.0% w/v, about 10.0 and about 15.0% w/v, about 10.0 and about 20.0% w/v, about 10 and about 25.0% w/v, about 10.0 and about 30.0% w/v, about 15.0 and about 20.0% w/v, about 15.0 and about 25.0% w/v, about 15.0 and about 30.0% w/v of the stabilizer. In certain embodiments, the liquid pharmaceutical composition comprises two, three, or four stabilizers in combination, wherein each individual stabilizer is present in an amount as described above, or the combination of stabilizers is present in an amount as described above.

According to certain embodiments, the stabilizer comprises a base. In other embodiments a liquid pharmaceutical composition comprises a stabilizer and further comprises a base, e.g., in order to provide the composition with a pre-defined pH. According to some embodiments, the base is selected from NaOH, NH₄OH, Ca(OH)₂, ammonium hydroxide, arginine, magnesium hydroxide, potassium hydroxide, meglumine, tromethamine (TRIS), triethylamine, ethylendiamine, diethylamine, ethanolamine, diisopropylethylamine, diazabicycloundecene or any combination thereof. The liquid pharmaceutical compositions may comprise between about 0.1 to about 30.0% w/v of a base. According to some embodiments, the liquid pharmaceutical composition comprises between about 0.1 to about 1.0% w/v, between about 1.0 to about 2.0% w/v, between about 2.0 to about 3.0% w/v, between about 3.0 to about 4.0% w/v, between about 4.0 to about 5.0% w/v, between about 5.0 to about 6.0% w/v, between about 6.0 to about 7.0% w/v, between about 8.0 to about 9.0% w/v, between about 9.0 to about 10.0% w/v, between about 10.0 to about 11.0% w/v, between about 11.0 to about 12.0% w/v, between about 12.0 to about 13.0% w/v, between about 13.0 to about 14.0% w/v, between about 14.0 to about 15.0% w/v, between about 15.0 to about 16.0% w/v, between about 16.0 to about 17.0% w/v, between about 17.0 to about 18.0% w/v, between about 18.0 to about 19.0% w/v, between about 19.0 to about 20.0% w/v, between about 20.0 to about 21.0% w/v, between about 21.0 to about 22.0% w/v, between about 22.0 to about 23.0% w/v, between about 23.0 to about 24.0% w/v, between about 24.0 to about 25.0% w/v, between about 25.0 to about 26.0% w/v, between about 26.0 to about 27.0% w/v, between about 27.0 to about 28.0% w/v, between about 28.0 to about 29.0% w/v, between about 29.0 to about 30.0% w/v, of a base.

In certain embodiments, the stabilizer comprises a base selected from the group consisting of arginine, NaOH, NH₄OH, tris(hydroxymethyl)aminomethane (TRIS), ethylendiamine, diethylamine, ethanolamine, diethanolamine, meglumine, triethanolamine, and any combination thereof. In certain embodiments, the base is selected from the group consisting of arginine, NH₄OH, ethylendiamine, diethylamine, ethanolamine, diethanolamine, meglumine, and a combination thereof. In certain embodiments, the base is selected from the group consisting of L-Arg, diethylamine, and a combination thereof. In certain embodiments, the base is selected from the group consisting of L-Arg, ethanolamine, and a combination thereof. In certain embodiments, the liquid pharmaceutical composition comprises between about 0.1% to about 30.0% w/v of the base. In certain embodiments, the liquid pharmaceutical composition comprises between about 1.5% to about 20.0% w/v of the base. For example, the liquid pharmaceutical composition can comprise between about 1.5 to about 5.0% w/v, between about 1.5 to about 10.0% w/v, between about 1.5 to about 15.0% w/v, about 1.5 to about 20.0% w/v, between about 1.5 to about 25.0% w/v, between about 3.0 to about 5.0% w/v, about 3.0 to about 10.0% w/v, between about 3.0 to about 15.0% w/v, between about 3.0 and about 20.0% w/v, about 3.0 to about 25.0% w/v, between about 3.0 to about 30.0% w/v, about 5.0 to about 10.0% w/v, between about 5.0 to about 15.0% w/v, between about 5.0 to about 20.0% w/v, between about 5.0 to about 25.0% w/v, between about 5.0 to about 30.0% w/v, between about 7.0 to about 10.0% w/v, between about 7.0 to about 15.0% w/v, between about 7.0 to about 20.0% w/v, between about 7.0 to about 25.0% w/v, about 7.0 to about 30.0% w/v, between about 10.0 to about 15.0% w/v, between about 10.0 to about 20.0% w/v, between about 10.0 to about 25.0% w/v, between about 10.0 to about 30.0% w/v, between about 15.0 to about 20.0% w/v, between about 15.0 to about 25.0% w/v, between about 15.0 to about 30.0% w/v of the base. In certain embodiments, the liquid pharmaceutical composition comprises two, three, or four stabilizers in combination. For example, in certain embodiments, the liquid pharmaceutical composition comprises arginine and an additional base selected from the group consisting of NH₄OH, ethylendiamine, diethylamine, ethanolamine, diethanolamine, meglumine. In certain embodiments, each individual stabilizer is present in an amount as described above, or the combination of stabilizers is present in an amount as described above. In certain embodiments, arginine is present in an amount of from about 7.0 to about 8.0% w/v and the additional base is present in an amount of from about 3.0 to about 8.0% w/v. In certain embodiments, the formulation comprises about 7.0 to about 8.0% (e.g., 7.2%) w/v L-Arg and about 3.0 to about 8.0% (e.g., about 5.4%) w/v diethylamine. In certain embodiments, the formulation comprises about 7.0 to about 8.0% (e.g., 7.2%) w/v L-Arg and about 2.0 to about 7.0% (e.g., about 4.5%) w/v ethanolamine.

In certain embodiments, the stabilizer includes one or more of polyethylene glycol (e.g., PEG-300, PEG-400, PEG-600), propylene glycol, choline, sodium or ammonium ions, an amino acid (e.g., Lys or His), benzyl alcohol, ethanol, group IIA metal complexes (e.g., Ca²⁺ salts such as CaCl₂ and Ca ascorbate), citric acid, lactic acid, or acetic acid, an electrophile (e.g., a Lewis acid such as Na⁺, K⁺, Ca²⁺, boron compounds, Fe³⁺, Al³⁺, Cu²⁺ and α-β unsaturated carbonyls such as maleic acid), phosphate buffer, Zn²⁺ ions, a reducing sugar such as glucose, sodium acetate, hydroxypropyl beta cyclodextrin, soluble beta cyclodextrin a medium chain triglyceride such as octanoic acid, mixed micelles glycocholate/lecithin, N-methyl pyrrolidone, dimethyl acetamide, soybean oil, sesame oil, castor oil, dimethyl sulfoxide, glycerine, tris buffer, ammonium acetate, or guanidine HCl. In certain embodiments, the stabilizer includes a surfactant such as those described herein, e.g., poloxamer, Tween-80, Tween-20, and Kolliphor.

According to some embodiments, the liquid pharmaceutical compositions further comprises an acid, e.g., in order to provide a composition with a pre-defined pH. According to some embodiments, the acid is selected from HCl, HBr, methanesulfonic acid, ascorbic acid, acetic acid, citric acid, or any combination thereof. The liquid pharmaceutical compositions may comprise between about 0.1 to about 30.0% w/v of an acid. According to some embodiments, the liquid pharmaceutical composition comprises between about 0.1 to about 1.0% w/v, between about 1.0 to about 2.0% w/v, between about 2.0 to about 3.0% w/v, between about 3.0 to about 4.0% w/v, between about 4.0 to about 5.0% w/v, between about 5.0 to about 6.0% w/v, between about 6.0 to about 7.0% w/v, between about 8.0 to about 9.0% w/v, between about 9.0 to about 10.0% w/v, between about 10.0 to about 11.0% w/v, between about 11.0 to about 12.0% w/v, between about 12.0 to about 13.0% w/v, between about 13.0 to about 14.0% w/v, between about 14.0 to about 15.0% w/v, between about 15.0 to about 16.0% w/v, between about 16.0 to about 17.0% w/v, between about 17.0 to about 18.0% w/v, between about 18.0 to about 19.0% w/v, between about 19.0 to about 20.0% w/v, between about 20.0 to about 21.0% w/v, between about 21.0 to about 22.0% w/v, between about 22.0 to about 23.0% w/v, between about 23.0 to about 24.0% w/v, between about 24.0 to about 25.0% w/v, between about 25.0 to about 26.0% w/v, between about 26.0 to about 27.0% w/v, between about 27.0 to about 28.0% w/v, between about 28.0 to about 29.0% w/v, between about 29.0 to about 30.0% w/v, of an acid.

The pH of the liquid pharmaceutical composition of the invention may be between about 4.5 to about 10 at about 25° C. According to some embodiments, the pH of the liquid pharmaceutical compositions is between about 4.5 to about 5 at about 25° C. According to some embodiments, the pH of the liquid pharmaceutical compositions is between about 5 to about 6 at about 25° C. According to some embodiments, the pH of the liquid pharmaceutical compositions is between about 6 to about 7 at about 25° C. According to some embodiments, the pH of the liquid pharmaceutical compositions is between about 7 to about 8 at about 25° C. According to some embodiments, the pH of the liquid pharmaceutical compositions is between about 8 to about 9 at about 25° C. According to some embodiments, the pH of the liquid pharmaceutical compositions is between about 9 to about 10 at about 25° C. According to some embodiments, the pH of the liquid pharmaceutical compositions is between about 4.5 to about 5.5 at about 25° C. According to some embodiments, the pH of the liquid pharmaceutical compositions is between about 5.5 to about 6.5 at about 25° C. According to some embodiments, the pH of the liquid pharmaceutical compositions is between about 6.5 to about 7.5 at about 25° C. According to some embodiments, the pH of the liquid pharmaceutical compositions is between about 7.5 to about 8.5 at about 25° C. According to some embodiments, the pH of the liquid pharmaceutical compositions is between about 8.5 to about 9.5 at about 25° C. According to some embodiments, the pH of the liquid pharmaceutical compositions is between about 9.5 to about 10 at about 25° C.

According to some embodiments, the liquid pharmaceutical composition further comprises a decarboxylase inhibitor. According to some embodiments, the decarboxylase inhibitor is selected from carbidopa, benserazide, methyldopa, 3′,4′,5,7-Tetrahydroxy-8-methoxyisoflavone, alpha-difluoromethyl-dopa, or any combination thereof. According to some embodiments, the decarboxylase inhibitor is carbidopa.

The liquid pharmaceutical composition of the invention may comprise between about 0.25 to about 3.0% w/v of a decarboxylase inhibitor, e.g., carbidopa. According to some embodiments, the liquid pharmaceutical compositions comprises between about 0.25 to about 0.5% w/v, between about 0.5 to about 0.75% w/v, between about 0.75 to about 1.0% w/v, between about 1.0 to about 1.25% w/v, between about 1.25 to about 1.5% w/v, between about 1.5 to about 1.75% w/v, between about 1.75 to about 2.0% w/v, between about 2.0 to about 2.25% w/v, between about 2.25 to about 2.5% w/v, between about 2.5 to about 2.75% w/v, between about 2.75 to about 3.0% w/v, between about 0.5 to about 1.0% w/v, between about 1.0 to about 1.5% w/v, between about 0.75 to about 1.4% w/v, between about 0.6 to about 0.9% w/v, between about 0.7 to about 0.8% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1.0% w/v, about 1.05% w/v, about 1.1% w/v, about 1.15% w/v, about 1.2% w/v, about 1.25% w/v, about 1.3% w/v, about 1.35% w/v, about 1.4% w/v, about 1.45% w/v, about 1.5% w/v, of a decarboxylase inhibitor, such as carbidopa.

According to some embodiments, the stabilizer comprises a buffer. According to some embodiments, the liquid pharmaceutical composition comprises a stabilizer and further comprises a buffer. According to some embodiments, the buffer is selected from citrate buffer, citric acid buffer, sodium acetate buffer, acetic acid buffer, tartaric acid buffer, phosphate buffer, succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, tromethamine (TRIS), or any combination thereof. The liquid pharmaceutical compositions may comprise between about 0.1 to about 30.0% w/v of a buffer. According to some embodiments, the liquid pharmaceutical composition comprises between about 0.1 to about 1.0% w/v, between about 1.0 to about 2.0% w/v, between about 2.0 to about 3.0% w/v, between about 3.0 to about 4.0% w/v, between about 4.0 to about 5.0% w/v, between about 5.0 to about 6.0% w/v, between about 6.0 to about 7.0% w/v, between about 8.0 to about 9.0% w/v, between about 9.0 to about 10.0% w/v, between about 10.0 to about 15.0% w/v, between about 15.0 to about 20.0% w/v, between about 20.0 to about 25.0% w/v, between about 25.0 to about 30.0% w/v of a buffer.

According to some embodiments, the liquid pharmaceutical composition further comprises an antioxidant. According to some embodiments, the antioxidant is selected from ascorbic acid or a salt thereof, a cysteine, a bisulfite or a salt thereof, glutathione, a tyrosinase inhibitor, a bivalent cation, such as a Cu⁺² chelator, butylated hydroxy toluene (BHT), beta hydroxy acid (BHA) tocopherol, gentisic acid, tocopherol, tocopherol derivative, such as tocopherol acetate or tocopherol succinate, thioglycerol, or any combination thereof.

According to some embodiments, the antioxidant is an ascorbic acid salt selected from sodium ascorbate, calcium ascorbate, potassium ascorbate, or any combination thereof. According to some embodiments, the antioxidant is a cysteine selected from L-cysteine, N-acetyl cysteine (NAC) or any combination thereof. According to some embodiments, the antioxidant is the bisulfite salt sodium metabisulfite. According to some embodiments, the antioxidant is the tyrosinase inhibitor captopril. According to some embodiments, the antioxidant is a Cu⁺² chelator is selected from Na₂-EDTA and Na₂-EDTA-Ca, or any combination thereof.

According to some embodiments, the antioxidant is selected from methimazole, quercetin, arbutin, aloesin, N-acetylglucosamine, retinoic acid, alpha-tocopheryl ferulate, Mg ascorbyl phosphate (MAP), substrate analogues, such as sodium benzoate, L-phenylalanine, dimercaptosuccinic acid, D-penicillamine, trientine-HCl, dimercaprol, clioquinol, sodium thiosulfate, triethylenetetramine, tetraethylenepentamine, curcumin, neocuproine, tannin, cuprizone, sulfite salts, such as sodium hydrogen sulfite or sodium metabisulfite, lipoic acid, CB4 (N-acetyl CysGlyProCys amide), CB3 (N-acetyl CysProCys amide), AD4 (N-acetyl cysteine amide), AD6 (N-acetylGluCysGly amide), AD7 (N-acetylCysGly amide), vitamin E, di-tert-butyl methyl phenol, tert-butyl-methoxyphenol, a polyphenol, a tocopherol, an ubiquinone, caffeic acid, or any combination thereof.

The liquid pharmaceutical compositions of the invention may comprise between about 0.05 to about 2.0% w/v of an antioxidant or a combination of antioxidants. According to some embodiments, the liquid pharmaceutical composition comprises between about 0.05 to about 0.1% w/v, about 0.1 to about 0.2% w/v, about 0.2 to about 0.3% w/v, about 0.3 to about 0.4% w/v, about 0.4 to about 0.5% w/v, about 0.5 to about 0.6% w/v, about 0.7 to about 0.8% w/v, about 0.8 to about 0.9% w/v, about 0.9 to about 1.0% w/v, about 1.0 to about 1.1% w/v, about 1.1 to about 1.2% w/v, about 1.2 to about 1.3% w/v, about 1.3 to about 1.4% w/v, about 1.4 to about 1.5% w/v, about 1.5 to about 1.6% w/v, about 1.6 to about 1.7% w/v, about 1.7 to about 1.8% w/v, about 1.8 to about 1.9% w/v, about 1.9 to about 2.0% w/v, about 0.75% w/v, about 0.8% w/v, about 0.85% w/v, about 0.9% w/v, about 0.95% w/v, about 1.0% w/v, about 1.05% w/v, about 1.1% w/v, about 1.15% w/v, about 1.2% w/v, of an antioxidant or a combination of antioxidants.

According to some embodiments, the liquid pharmaceutical composition comprises a combination of two antioxidants, wherein each antioxidant is present in an amount of between about 0% w/v and about 2% w/v and wherein the total amount of antioxidant is present in an amount of between about 0% w/v to about 2% w/v. According to some embodiments, the liquid pharmaceutical composition comprises between about 0.05 to about 0.1% w/v, about 0.1 to about 0.2% w/v, about 0.2 to about 0.3% w/v, about 0.3 to about 0.4% w/v, about 0.4 to about 0.5% w/v, about 0.5 to about 0.6% w/v, about 0.7 to about 0.8% w/v, about 0.8 to about 0.9% w/v, about 0.9 to about 1.0% w/v, about 1.0 to about 1.1% w/v, about 1.1 to about 1.2% w/v, about 1.2 to about 1.3% w/v, about 1.3 to about 1.4% w/v, about 1.4 to about 1.5% w/v, about 1.5 to about 1.6% w/v, about 1.6 to about 1.7% w/v, about 1.7 to about 1.8% w/v, about 1.8 to about 1.9% w/v, about 1.9 to about 2.0% w/v of a first antioxidant and between about 0.05 to about 0.1% w/v, about 0.1 to about 0.2% w/v, about 0.2 to about 0.3% w/v, about 0.3 to about 0.4% w/v, about 0.4 to about 0.5% w/v, about 0.5 to about 0.6% w/v, about 0.7 to about 0.8% w/v, about 0.8 to about 0.9% w/v, about 0.9 to about 1.0% w/v, about 1.0 to about 1.1% w/v, about 1.1 to about 1.2% w/v, about 1.2 to about 1.3% w/v, about 1.3 to about 1.4% w/v, about 1.4 to about 1.5% w/v, about 1.5 to about 1.6% w/v, about 1.6 to about 1.7% w/v, about 1.7 to about 1.8% w/v, about 1.8 to about 1.9% w/v, about 1.9 to about 2.0% w/v of a second antioxidant, wherein the combination of the first and second antioxidants is present in an amount of no more that about 2% w/v of the liquid pharmaceutical composition. In certain embodiments, the first and second antioxidants comprise N-acetyl cysteine (NAC) and ascorbic acid or a salt thereof. In certain embodiments, the liquid pharmaceutical composition comprises 0, about 0.25%, about 0.5%, about 0.75%, about 1%%, about 1.25%, about 1.5%, about 1.75%, or about 2% NAC and 0, about 0.25%, about 0.5%, about 0.75%, about 1%, about 1.25%, about 1.5%, about 1.75%, or about 2% ascorbic acid or a salt thereof, wherein the liquid pharmaceutical compositions comprises no more than 2% of the combination of NAC and ascorbic acid or a salt thereof. In certain embodiments, the liquid pharmaceutical composition comprises about 1% NAC and no ascorbic acid, about 1% NAC and about 1% ascorbic acid or a salt thereof, about 2% NAC and no ascorbic acid, or about 2% ascorbic acid or a salt thereof and no NAC.

According to some embodiments, the liquid pharmaceutical composition further comprises a catechol-O-methyltransferase (COMT) inhibitor. According to some embodiments, the COMT inhibitor is selected from entacapone, tolcapone, opicapone or any combination thereof. According to some embodiments, the liquid pharmaceutical composition comprises between about 0.1 to about 5.0% w/v of a COMT inhibitor. According to some embodiments, the liquid pharmaceutical composition comprises between about 0.1 to about 1.0% w/v of a COMT inhibitor. According to some embodiments, the liquid pharmaceutical composition comprises between about 1.0 to about 2.0% w/v of a COMT inhibitor. According to some embodiments, the liquid pharmaceutical composition comprises between about 2.0 to about 3.0% w/v of a COMT inhibitor. According to some embodiments, the liquid pharmaceutical composition comprises between about 3.0 to about 4.0% w/v of a COMT inhibitor. According to some embodiments, the liquid pharmaceutical composition comprises between about 4.0 to about 5.0% w/v of a COMT inhibitor. According to some embodiments, the liquid pharmaceutical composition may be administered concomitantly with a COMT inhibitor.

According to some embodiments, the liquid pharmaceutical composition further comprises a monoamine oxidase (MAO) inhibitor. The MAO inhibitor may be a MAO-A inhibitor or a MAO-B inhibitor. According to some embodiments, the liquid pharmaceutical composition comprises between about 0.1 to about 5.0% w/v of a MAO inhibitor. According to some embodiments, the liquid pharmaceutical composition comprises between about 0.1 to about 1.0% w/v of a MAO inhibitor. According to some embodiments, the liquid pharmaceutical composition comprises between about 1.0 to about 2.0% w/v of a MAO inhibitor. According to some embodiments, the liquid pharmaceutical composition comprises between about 2.0 to about 3.0% w/v of a MAO inhibitor. According to some embodiments, the liquid pharmaceutical composition comprises between about 3.0 to about 4.0% w/v of a MAO inhibitor. According to some embodiments, the liquid pharmaceutical composition comprises between about 4.0 to about 5.0% w/v of a MAO inhibitor. According to some embodiments, the MAO inhibitor is selected from moclobemide, rasagiline, selegiline, safinamide, or any combination thereof. According to some embodiments, the liquid pharmaceutical composition may be administered concomitantly with a MAO inhibitor.

According to some embodiments, the liquid pharmaceutical composition further comprises a surfactant. According to some embodiments, the surfactant is selected from Tween-80, Tween-60, Tween-40, Tween-20, Tween-65, Tween-85, Span 20, Span 40, Span 60, Span 80, Span 85, polyoxyl 35 castor oil (Cremophor EL), polyoxyethylene-660-hydroxystearate (macrogol 660), or Poloxamer 188 (Pluronic® F-68), or any combination thereof. The liquid pharmaceutical composition of the invention may include between about 0.1 to about 3.0% w/v of a surfactant or combination of two or more surfactants. According to some embodiments, the liquid pharmaceutical composition comprises between about 0.1 to about 0.2% w/v, between about 0.2 to about 0.3% w/v, between about 0.3 to about 0.4% w/v, between about 0.4 to about 0.5% w/v, between about 0.5 to about 0.6% w/v, between about 0.6 to about 0.7% w/v, between about 0.7 to about 0.8% w/v, between about 0.8 to about 0.9% w/v, between about 0.9 to about 1.0% w/v, between about 1.0 to about 1.5% w/v, between about 1.5 to about 2.0% w/v, between about 2.0 to about 2.5% w/v, between about 2.5 to about 3.0% w/v of a surfactant or combination of two or more surfactants.

The liquid pharmaceutical composition may further comprise an additional pharmaceutically acceptable excipient, such as N-methylpyrrolidone (NMP), polyvinylpyrrolidone (PVP), propylene glycol, a preservative, a pharmaceutically acceptable vehicle, a stabilizer, a dispersing agent, a suspending agent, an amino sugar, a calcium chelator, protease inhibitors, or any combination thereof. The liquid pharmaceutical composition of the invention may comprise between about 5.0 to about 80.0% w/v or an additional pharmaceutically acceptable excipient, e.g., a solvent, a buffer or any other co-solvent.

According to some embodiments, the liquid pharmaceutical composition of the invention comprises between about 5.0 to about 10.0% w/v, between about 10.0 to about 15.0% w/v, between about 15.0 to about 20.0% w/v, between about 20.0 to about 25.0% w/v, between about 25.0 to about 30.0% w/v, between about 30.0 to about 35.0% w/v, between about 35.0 to about 40.0% w/v, between about 40.0 to about 45.0% w/v, between about 45.0 to about 50.0% w/v, between about 50.0 to about 55.0% w/v, between about 55.0 to about 60.0% w/v, between about 60.0 to about 65.0% w/v, between about 65.0 to about 70.0% w/v, between about 70.0 to about 75.0% w/v, between about 75.0 to about 80.0% w/v of a solvent, a buffer or any other co-solvent.

It is noted that any one, or any combination, of any of the components disclosed herein may be added to the liquid pharmaceutical composition of the invention.

The liquid pharmaceutical compositions of the invention may be in the form of a solution, gel, cream, emulsion, or suspension. According to some embodiments, the liquid pharmaceutical compositions of the invention may be dried to provide a solid, e.g., by lyophilization, wherein the dried material, e.g., the lyophilizate, may be constituted to provide a liquid composition, e.g., by the addition of a solvent, e.g., water. Antioxidants, surfactants and the like may also be added when the dried composition is constituted. According to some embodiments, the dried composition is reconstituted using a dedicated solution comprising, e.g., a solvent, an antioxidant, a surfactant and any other required excipients. According to some embodiments, the liquid pharmaceutical composition of the invention is an aqueous composition.

The liquid pharmaceutical compositions of the invention may be formulated for any suitable route of administration, e.g., for parenteral administration, e.g., by bolus administration or continuous administration. The liquid pharmaceutical composition of the invention may be formulated for subcutaneous, transdermal, intradermal, transmucosal, intravenous, intraarterial, intramuscular, intraperitoneal, intratracheal, intrathecal, intraduodenal, intrapleural, intranasal, sublingual, buccal, intestinal, intraduodenally, rectal, intraocular, or oral administration. The compositions may also be formulated for inhalation, or for direct absorption through mucous membrane tissues.

Further embodiments of the invention are directed to a process for preparing a liquid pharmaceutical composition, wherein said process comprises:
mixing a levodopa-tyrosine (LD-Tyr) conjugate of formula (II):

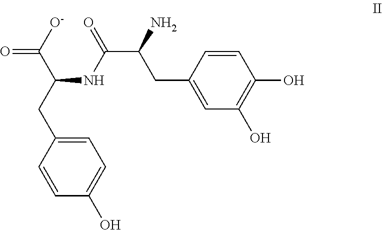 [SMILES: N[C@@H](Cc1ccc(O)c(O)c1)C(=O)N[C@@H](Cc1ccc(O)cc1)C(=O)[O-]]

in a pharmaceutically acceptable salt form with at least one solvent and/or stabilizer, thereby forming a solution, gel, cream, emulsion, or suspension; and adjusting the pH of the solution, gel, cream, emulsion, or suspension, to a physiologically acceptable pH value, thereby providing the liquid pharmaceutical composition.

According to some embodiments, the process comprises mixing an LD-Tyr compound of Formula (II) in a pharmaceutically acceptable salt form with at least one stabilizer, thereby forming a solution. According to some embodiments, the process comprises mixing an LD-Tyr compound of Formula (II) in a pharmaceutically acceptable solid salt form with at least one stabilizer. According to some embodiments, the process of the invention includes further mixing the LD-Tyr compound of Formula (II) with any additional active pharmaceutical ingredients and/or pharmaceutically acceptable excipients, as detailed regarding the liquid pharmaceutical composition of the invention. In certain embodiments, the mixing is performed without heating. In certain embodiments, the mixing is performed at room temperature.

According to some embodiments, the process comprises mixing a salt form of an LD-Tyr with at least one solvent and/or stabilizer, wherein the salt is a TFA salt, an HCl salt fumaric acid salt, lactate salt, maleic acid salt, gluceptic acid salt, phosphoric acid salt, sulfuric acid salt, HBr salt, nitric acid salt, acetic acid salt, propionic acid salt, hexanoic acid salt, cyclopentanepropionic acid salt, glycolic acid salt, pyruvic acid salt, lactic acid salt, hippuric acid salt, methanesulfonic acid salt, ascorbic acid salt, malonic acid salt, oxalic acid salt, maleic acid salt, tartaric acid salt, citric acid salt, succinic acid salt, benzoic acid salt, cinnamic acid salt, a sulfonic acid salt, lauryl sulfuric acid salt, gluconic acid salt, glutamic acid salt, hydroxynaphthoic acid salt, salicylic acid salt, stearic acid salt, muconic acid salt, an alkali metal salt, such as lithium salt, sodium salt or potassium salt, an alkaline earth metal salt, such as calcium salt or magnesium salt, an aluminum salt, an ethanolamine salt, diethanolamine salt, triethanolamine salt, N-methylglucamine salt, dicyclohexylamine salt, adipate salt, alginate salt, ascorbate salt, aspartate salt, benzenesulfonate salt, bisulfate salt, borate salt, butyrate salt, camphorate butyrate salt, camphorsulfonate butyrate salt, digluconate butyrate salt, dodecylsulfate butyrate salt, ethanesulfonate butyrate salt, glucoheptonate butyrate salt, glycerophosphate butyrate salt, gluconate butyrate salt, hemisulfate butyrate salt, heptanoate butyrate salt, hydroiodide butyrate salt, 2-hydroxy-ethanesulfonate butyrate salt, lactobionate butyrate salt, laurate butyrate salt, methanesulfonate butyrate salt, 2-naphthalenesulfonate butyrate salt, nicotinate butyrate salt, oleate butyrate salt, palmitate butyrate salt, pamoate butyrate salt, pectinate butyrate salt, persulfate butyrate salt, 3-phenylpropionate butyrate salt, phosphate butyrate salt, picrate butyrate salt, pivalate butyrate salt, tartrate butyrate salt, thiocyanate butyrate salt, p-toluenesulfonate butyrate salt, undecanoate butyrate salt, valerate salts, or any combination thereof.

Further embodiments of the invention are directed to a liquid pharmaceutical composition prepared according to the process of the invention.

Some embodiments of the invention are directed to a liquid pharmaceutical composition in which the LD-Tyr compound, an enantiomer, diastereomer, racemate, ion, zwitterion, pharmaceutically acceptable salt thereof, or any combination thereof has a solubility of between about 100 to about 1000 mg/L at a physiologically acceptable pH. According to some embodiments, the solubility of the LD-Tyr compound, an enantiomer, diastereomer, racemate, ion, zwitterion, pharmaceutically acceptable salt thereof, or any combination thereof, is between about 100 to about 200 mg/L, between about 200 to about 300 mg/L, between about 300 to about 400 mg/L, between about 400 to about 500 mg/L, between about 500 to about 600 mg/L, between about 600 to about 700 mg/L, between about 700 to about 800 mg/L, between about 800 to about 900 mg/L, between about 900 to about 1000 mg/L, at a physiologically acceptable pH.

Further embodiments of the invention are directed to a method of treating neurodegenerative conditions and/or conditions characterized by reduced levels of dopamine in the brain, wherein the method comprises administering a liquid pharmaceutical composition, wherein the liquid pharmaceutical composition comprises a stabilizer and a levodopa-tyrosine (LD-Tyr) conjugate of formula (II):

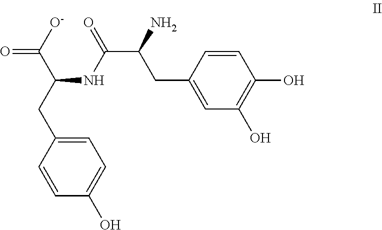 [SMILES: N[C@@H](Cc1ccc(O)c(O)c1)C(=O)N[C@@H](Cc1ccc(O)cc1)C(=O)[O-]]

an enantiomer, diastereomer, racemate, ion, zwitterion, pharmaceutically acceptable salt thereof, or any combination thereof.

According to some embodiments, neurodegenerative conditions and/or conditions characterized by reduced levels of dopamine in the brain are selected from Parkinson's disease, secondary parkinsonism, Huntington's disease, Parkinson's like syndrome, progressive supranuclear palsy (PSP), multiple system atrophy (MSA), amyotrophic lateral sclerosis (ALS), Shy-Drager syndrome, dystonia, Alzheimer's disease, Lewy body dementia (LBD), akinesia, bradykinesia, and hypokinesia, conditions resulting from brain injury, including carbon monoxide or manganese intoxication, conditions associated with a neurological disease or disorder, including alcoholism, opiate addiction, and erectile dysfunction. According to some embodiments, the neurodegenerative condition and/or condition characterized by reduced levels of dopamine in the brain is Parkinson's disease.

According to some embodiments, the method of the invention comprises administering the LD-Tyr compound of Formula (II), an enantiomer, diastereomer, racemate, ion, zwitterion, pharmaceutically acceptable salt thereof, or any combination thereof, or any combination of two or more LD-Tyr enantiomers, diastereomers, racemates, ions, zwitterions, pharmaceutically acceptable salts thereof, or any combination thereof, concomitantly with an additional active ingredient, such as a decarboxylase inhibitor, e.g., carbidopa, a COMT inhibitor, a MAO inhibitor, or any combination thereof. According to some embodiments, the LD-Tyr compound is administered together with a decarboxylase inhibitor, e.g., carbidopa, wherein the LD-Tyr compound and the decarboxylase inhibitor are administered in a single formulation.

According to some embodiments, the method of the invention comprises administering the liquid pharmaceutical composition substantially continuously. According to some embodiments, the liquid pharmaceutical composition is administered subcutaneously. According to some embodiments, the liquid pharmaceutical composition is administered subcutaneously via a designated pump device.

Embodiments of a designated pump may be, for example, any of the pump embodiments disclosed in U.S. 62/529,784, U.S. 62/576,362, PCT/IB2018/054962, U.S. Ser. No. 16/027,804, U.S. Ser. No. 16/027,710, U.S. Ser. No. 16/351,072, U.S. Ser. No. 16/351,076, U.S. Ser. No. 16/351,061, U.S. Ser. No. 29/655,583, U.S. Ser. No. 29/655,587, U.S. Ser. No. 29/655,589, U.S. Ser. No. 29/655,591, U.S. Ser. No. 29/655,592, U.S. Ser. No. 29/655,594, U.S. Ser. No. 29/655,597, U.S. 62/851,903, and U.S. Ser. No. 29/723,714, all of which are incorporated herein by reference in their entirety.

According to some embodiments, the method of the invention comprises administering the liquid pharmaceutical composition at one site, two sites, or three or more sites, wherein the position of the sites may be changed at any appropriate, possibly pre-determined, intervals. Once administered via a specific site, according to some embodiments, the administration via the same site, or the vicinity of that site, may be only after a, possibly predefined, period of time. According to some embodiments, the position of any one of the sites is changed after 12, 24, 36, 48, 60 or 72 hours. According to some embodiments, the position of the site is changed after 4, 5, 6 or 7 days. According to some embodiments, the position of the site is changed after two, three or four weeks. According to some embodiments, the position of the site is changed when required or desired, e.g., according to subjective data received from the patient and/or according to objective data received, e.g., from sensors located at, or in the vicinity of, the injection site(s).

According to some embodiments, the administrated volume and/or the administration rate is identical in all or at least two of the sites. According to other embodiments, the administration rate and/or administrated volume differ from site to site. Each site may be controlled independently or otherwise, all sites may be controlled dependently on one another.

According to some embodiments, the method of the invention comprises subcutaneously administrating between about 1 to about 15 ml of the liquid pharmaceutical composition of the invention over the course of 24 hours. According to some embodiments, the method of invention comprises subcutaneously administrating between about 1 to about 2, between about 2 to about 3, between about 3 to about 4, between about 4 to about 5, between about 5 to about 6, between about 6 to about 7, between about 7 to about 8, between about 8 to about 9, between about 9 to about 10, between about 10 to about 11, between about 11 to about 12, between about 12 to about 13, between about 13 to about 14, between about 14 to about 15 ml over the course of 24 hours.

It is noted that the administration rate may be constant over the course of 24 hours or may change over the course of 24 hours. For instance, according to some embodiments, there may be a certain rate for high activity/day hours and a different rate for low activity/night hours. The high activity/day hours may be, e.g., about 15, about 16, about 17, about 18 or about 19 hours, while the low activity night hours may be about 9, about 8, about 7, about 6 or about 5 hours, respectively. According to some embodiments, the high activity/day rate is implemented for about 18 hours, while the low activity/night rate is implemented for about 6 hours. According to some embodiments, the high activity/day rate is implemented for about 16 hours, while the low activity/night rate is implemented for about 8 hours.

According to some embodiments, the administration rate is constant over the course of 24 hours. According to some embodiments, the liquid pharmaceutical formulation is administered for a certain period of time in each 24 hours, e.g., 8 hours a day, 9 hours a day, 10 hours a day, 11 hours a day, 12 hours a day, 13 hours a day, 14 hours a day, 15 hours a day, 16 hours a day, 17 hours a day, 18 hours a day, 19 hours a day, 20 hours a day, 21 hours a day, 22 hours a day, or 23 hours a day. According to some embodiments, the number of hours of administration per day may be constant over the course of a certain number of days, e.g., 7 days, 14 days, 21 days, 28 days, two months, three months, four months, five months, six months, seven months, eight months, nine months, ten months, eleven months, one year, two years, three years, four years, or more.

According to some embodiments, the number of hours of administration per day may vary from day to day, according to the patient's condition, aa caregiver's or physician's decision, input from sensors, and the like. It is further noted that while only whole hours are specifically mentioned, any parts of hours, days, months, etc., are possible for administration, e.g., 16.5 hours a day, 7.5 days, and the like.

The administration rate may be between about 0.01 mL/site/hour to about 1 mL/site/hour. According to some embodiments, the administration rate is between about 0.01-0.02 mL/site/hour. According to some embodiments, the administration rate is between about 0.02-0.03 mL/site/hour. According to some embodiments, the administration rate is between about 0.03-0.04 mL/site/hour. According to some embodiments, the administration rate is between about 0.04-0.05 mL/site/hour. According to some embodiments, the administration rate is between about 0.05-0.06 mL/site/hour. According to some embodiments, the administration rate is between about 0.06-0.07 mL/site/hour. According to some embodiments, the administration rate is between about 0.07-0.08 mL/site/hour. According to some embodiments, the administration rate is between about 0.08-0.09 mL/site/hour. According to some embodiments, the administration rate is between about 0.09-0.1 mL/site/hour. According to some embodiments, the administration rate is between about 0.1-0.15 mL/site/hour. According to some embodiments, the administration rate is between about 0.15-0.2 mL/site/hour. According to some embodiments, the administration rate is between about 0.2-0.25 mL/site/hour. According to some embodiments, the administration rate is between about 0.25-0.3 mL/site/hour. According to some embodiments, the administration rate is between about 0.3-0.35 mL/site/hour. According to some embodiments, the administration rate is between about 0.35-0.4 mL/site/hour. According to some embodiments, the administration rate is between about 0.4-0.45 mL/site/hour. According to some embodiments, the administration rate is between about 0.45-0.5 mL/site/hour. According to some embodiments, the administration rate is between about 0.5-0.55 mL/site/hour. According to some embodiments, the administration rate is between about 0.55-0.6 mL/site/hour. According to some embodiments, the administration rate is between about 0.6-0.65 mL/site/hour. According to some embodiments, the administration rate is between about 0.65-0.7 mL/site/hour. According to some embodiments, the administration rate is between about 0.7-0.75 mL/site/hour. According to some embodiments, the administration rate is between about 0.75-0.8 mL/site/hour. According to some embodiments, the administration rate is between about 0.8-0.85 mL/site/hour. According to some embodiments, the administration rate is between about 0.85-0.9 mL/site/hour. According to some embodiments, the administration rate is between about 0.9-0.95 mL/site/hour. According to some embodiments, the administration rate is between about 0.95-1.0 mL/site/hour.

According to some embodiments, the administration rate in the low activity/night hours is between about 0.01-0.15 mL/site/hour. According to some embodiments, the administration rate in the low activity/night hours is between about 0.01-0.02 mL/site/hour. According to some embodiments, the administration rate in the low activity/night hours is between about 0.02-0.03 mL/site/hour. According to some embodiments, the administration rate in the low activity/night hours is between about 0.03-0.04 mL/site/hour. According to some embodiments, the administration rate in the low activity/night hours is between about 0.04-0.05 mL/site/hour. According to some embodiments, the administration rate in the low activity/night hours is between about 0.05-0.06 mL/site/hour. According to some embodiments, the administration rate in the low activity/night hours is between about 0.06-0.07 mL/site/hour. According to some embodiments, the administration rate in the low activity/night hours is between about 0.07-0.08 mL/site/hour. According to some embodiments, the administration rate in the low activity/night hours is between about 0.08-0.09 mL/site/hour. According to some embodiments, the administration rate in the low activity/night hours is between about 0.09-0.1 mL/site/hour. According to some embodiments, the administration rate in the low activity/night hours is between about 0.1-0.11 mL/site/hour. According to some embodiments, the administration rate in the low activity/night hours is between about 0.11-0.12 mL/site/hour. According to some embodiments, the administration rate in the low activity/night hours is between about 0.12-0.13 mL/site/hour. According to some embodiments, the administration rate in the low activity/night hours is between about 0.13-0.14 mL/site/hour. According to some embodiments, the administration rate in the low activity/night hours is between about 0.14-0.15 mL/site/hour. According to some embodiments, the administration rate in the low activity/night hours is about 0.04 mL/site/hour.

According to some embodiments, the administration rate in the high activity/day hours is between about 0.15-1.0 mL/site/hour. According to some embodiments, the administration rate in the high activity/day hours is between about 0.15-0.2 mL/site/hour. According to some embodiments, the administration rate in the high activity/day hours is between about 0.2-0.25 mL/site/hour. According to some embodiments, the administration rate in the high activity/day hours is between about 0.25-0.3 mL/site/hour. According to some embodiments, the administration rate in the high activity/day hours is between about 0.3-0.35 mL/site/hour. According to some embodiments, the administration rate in the high activity/day hours is between about 0.35-0.4 mL/site/hour. According to some embodiments, the administration rate in the high activity/day hours is between about 0.4-0.45 mL/site/hour. According to some embodiments, the administration rate in the high activity/day hours is between about 0.45-0.5 mL/site/hour. According to some embodiments, the administration rate in the high activity/day hours is between about 0.5-0.55 mL/site/hour. According to some embodiments, the administration rate in the high activity/day hours is between about 0.55-0.6 mL/site/hour. According to some embodiments, the administration rate in the high activity/day hours is between about 0.6-0.65 mL/site/hour. According to some embodiments, the administration rate in the high activity/day hours is between about 0.65-0.7 mL/site/hour. According to some embodiments, the administration rate in the high activity/day hours is between about 0.7-0.75 mL/site/hour. According to some embodiments, the administration rate in the high activity/day hours is between about 0.75-0.8 mL/site/hour. According to some embodiments, the administration rate in the high activity/day hours is between about 0.8-0.85 mL/site/hour. According to some embodiments, the administration rate in the high activity/day hours is between about 0.85-0.9 mL/site/hour. According to some embodiments, the administration rate in the high activity/day hours is between about 0.9-0.95 mL/site/hour. According to some embodiments, the administration rate in the high activity/day hours is between about 0.95-1.0 mL/site/hour. According to some embodiments, the administration rate in the high activity/day hours is about 0.32 mL/site/hour.

It is further noted that the administrated volume and/or administration rate may be constant throughout the treatment, or may vary during different hours of the day, between different days, weeks or months of treatment, and the like. According to some embodiments, the patient is monitored, e.g., independently, by a caretaker, or electronically, e.g., by sensors, possibly found in a dedicated device, e.g., a watch-like device, the administration pump, and the like. According to such embodiments, the administration volume and/or rate are determined according to data received from such monitoring.

Some embodiments are directed to a method for administering a bolus subcutaneous injection of the liquid pharmaceutical composition of the invention. According to some embodiments, the bolus injection comprises between about 0.5 to about 2.0 mL/Kg of the liquid pharmaceutical composition. According to some embodiments, the bolus injection comprises between about 0.5 to about 0.75 mL/Kg of the liquid pharmaceutical composition. According to some embodiments, the bolus injection comprises between about 0.75 to about 1.0 mL/Kg of the liquid pharmaceutical composition. According to some embodiments, the bolus injection comprises between about 1.0 to about 1.25 mL/Kg of the liquid pharmaceutical composition. According to some embodiments, the bolus injection comprises between about 1.25 to about 1.5 mL/Kg of the liquid pharmaceutical composition. According to some embodiments, the bolus injection comprises between about 1.5 to about 1.75 mL/Kg of the liquid pharmaceutical composition. According to some embodiments, the bolus injection comprises between about 1.75 to about 2.0 mL/Kg of the liquid pharmaceutical composition. According to some embodiments, the bolus injection comprises between about 0.75 to about 1.25 mL/Kg of the liquid pharmaceutical composition. According to some embodiments, the bolus injection comprises about 1.0 mL/Kg of the liquid pharmaceutical composition.

The bolus subcutaneous injection may be administered at any time point in relation to any possible continuous subcutaneous administrations, e.g., prior to, during, or after the continuous administration.

According to some embodiments, the administered dose may be doubled, tripled or more, by using more than one pump, more than one injection site for each pump, and the like.

According to some embodiments, the liquid pharmaceutical compositions are administered for a defined period of time, e.g., days, weeks, months, or years. According to some embodiments, the liquid pharmaceutical compositions are administered endlessly, for the treatment of a chronic condition.

Further embodiments of the invention are directed to a liquid pharmaceutical composition for use in the treatment of neurodegenerative conditions and/or conditions characterized by reduced levels of dopamine in the brain, wherein the liquid pharmaceutical composition comprises a stabilizer and a levodopa-tyrosine (LD-Tyr) conjugate of formula (II):

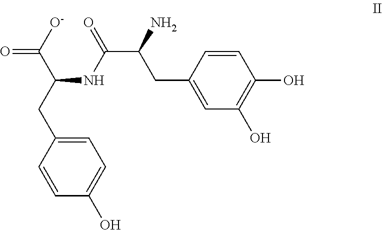 [SMILES: N[C@@H](Cc1ccc(O)c(O)c1)C(=O)N[C@@H](Cc1ccc(O)cc1)C(=O)[O-]]

an enantiomer, diastereomer, racemate, ion, zwitterion, pharmaceutically acceptable salt thereof, or any combination thereof.

According to some embodiments, the liquid pharmaceutical composition is for use in the treatment of Parkinson's disease, secondary parkinsonism, Huntington's disease, Parkinson's like syndrome, progressive supranuclear palsy (PSP), multiple system atrophy (MSA), amyotrophic lateral sclerosis (ALS), Shy-Drager syndrome, dystonia, Alzheimer's disease, Lewy body dementia (LBD), akinesia, bradykinesia, and hypokinesia, conditions resulting from brain injury, including carbon monoxide or manganese intoxication, conditions associated with a neurological disease or disorder, including alcoholism, opiate addiction, and erectile dysfunction. Certain embodiments of the invention are directed to the liquid pharmaceutical composition of the invention in the treatment of Parkinson's disease.

The composition for use according to the invention may include any of the additional materials, the amounts of any of the materials, as detailed herein regarding the embodiments of the composition of the invention. Further, the form, pH, and the like, of the compositions for use according to the invention may be as detailed herein regarding the embodiments of the composition of the invention. In addition, the composition of the invention may be used together with a COMT inhibitor, MAO inhibitor, or any other active ingredient, as detailed herein.

Industrial Applicability: In the present invention, the levodopa prodrug compound and the stabilized formulations described herein are useful for the prevention or treatment of neurodegenerative diseases and/or diseases or symptoms caused by a decrease in dopamine concentration in the brain, such as Parkinson's disease and related symptoms. Therefore, the present invention has a high utility value in the pharmaceutical industry.

Unless explicitly stated, the method embodiments described herein are not constrained to a particular order or sequence. Additionally, some of the described method embodiments or elements thereof can occur or be performed simultaneously, at the same point in time, or concurrently.

It is appreciated that certain features of the invention may also be provided in combination in a single embodiment. Conversely, various elements of the invention that are, for brevity, described in the context of a single embodiment, may also be provided separately or in any suitable sub-combination or as suitable in any other described embodiment of the invention. Further, certain features described in the context of various embodiments are not to be considered essential features of those embodiments, unless the embodiment is inoperative without those elements.

Various embodiments and aspects of the present invention as delineated hereinabove and as claimed in the claims section below may be supported by the following examples; however, they are not to be limited by the examples.

### EXAMPLES

### Part I: Prodrugs

### Example 1: Production of (2S)-2-[[(2S)-2-aminopropanoyl]amino]-3-(3-hydroxy-4-phosphonooxyphenyl) propanoic acid

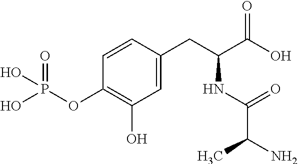 [SMILES: C[C@H](N)C(=O)N[C@@H](Cc1ccc(OP(=O)(O)O)c(O)c1)C(=O)O]

(2S)-3-[4-bis (phenylmethoxy)phosphoryloxy-3-phenylmethoxyphenyl]-2-[[(2S)-2-(phenylmethoxycarbonylamino)propanoyl]amino] benzyl propanoate (551 mg) was dissolved in a mixed solvent of ethanol (2 mL) and tetrahydrofuran (2 mL), and palladium/carbon (hydrous) (69 mg) was added, and the mixture was stirred under a hydrogen atmosphere at room temperature for 7 hours. The reaction mixture was filtered through a membrane filter (cellulose acetate) to remove insoluble matter. The insoluble matter was washed with water/ethanol (2:1, 12 mL), and the filtrate was distilled under a reduced pressure until the filtrate was reduced to about 1 mL, and freeze-drying was performed, and then, the title compound (214 mg, yield: 100%) as a white powder was obtained.

MS (ESI); m/z 349.1 [M+H]+

### Examples 2-19 and 120-131

The corresponding starting compounds were respectively treated in the same manner as in Example 1 to obtain the compounds shown in Table 2 below. Some of the compounds shown in Table 2 below can be obtained in the same manner as above example.

TABLE 2

| Example | Structural formula | Physical property values and the like |
|---|---|---|
| 2 | 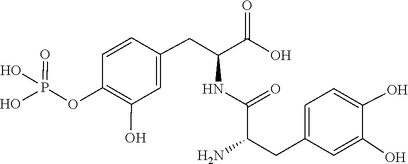 [SMILES: N[C@@H](Cc1ccc(O)c(O)c1)C(=O)N[C@@H](Cc1ccc(OP(=O)(O)O)c(O)c1)C(=O)O] | MS (ESI); m/z 457.4 [M + H]+ |
| 3 | 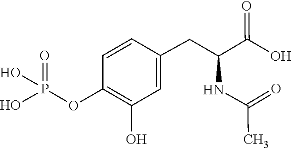 [SMILES: CC(=O)N[C@@H](Cc1ccc(OP(=O)(O)O)c(O)c1)C(=O)O] | MS (ESI); m/z 320.1 [M + H]+ |
| 4 | 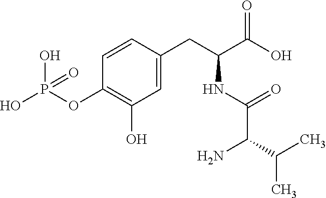 [SMILES: CC(C)[C@H](N)C(=O)N[C@@H](Cc1ccc(OP(=O)(O)O)c(O)c1)C(=O)O] | MS (ESI); m/z 375.2 [M − H]− |
| 5 | 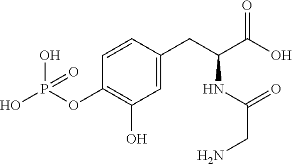 [SMILES: NCC(=O)N[C@@H](Cc1ccc(OP(=O)(O)O)c(O)c1)C(=O)O] | MS (ESI); m/z 335.3 [M + H]+ |
| 6 | 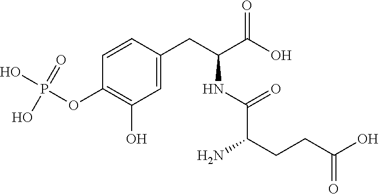 [SMILES: N[C@@H](CCC(=O)O)C(=O)N[C@@H](Cc1ccc(OP(=O)(O)O)c(O)c1)C(=O)O] | MS (ESI); m/z 405.0 [M − H]− |
| 7 | 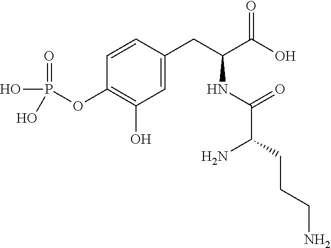 [SMILES: NCCC[C@H](N)C(=O)N[C@@H](Cc1ccc(OP(=O)(O)O)c(O)c1)C(=O)O] | MS (ESI); m/z 392.3 [M + H]+ |
| 8 | 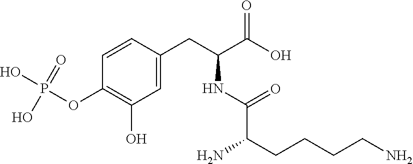 [SMILES: NCCCC[C@H](N)C(=O)N[C@@H](Cc1ccc(OP(=O)(O)O)c(O)c1)C(=O)O] | MS (ESI); m/z 406.3 [M + H]+ |
| 9 | 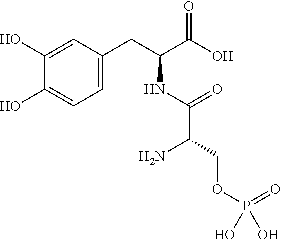 [SMILES: N[C@@H](COP(=O)(O)O)C(=O)N[C@@H](Cc1ccc(O)c(O)c1)C(=O)O] | MS (ESI); m/z 365.1 [M + H]+ |
| 10 | 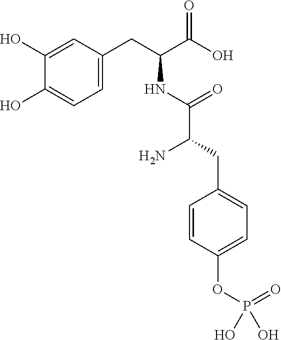 [SMILES: N[C@@H](Cc1ccc(OP(=O)(O)O)cc1)C(=O)N[C@@H](Cc1ccc(O)c(O)c1)C(=O)O] | MS (ESI); m/z 441.1 [M + H]+ |
| 11 | 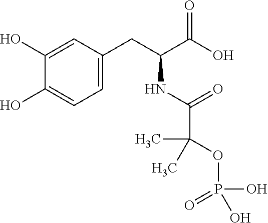 [SMILES: CC(C)(OP(=O)(O)O)C(=O)N[C@@H](Cc1ccc(O)c(O)c1)C(=O)O] | MS (ESI); m/z 364.1 [M + H]+ |
| 12 | 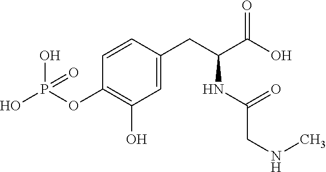 [SMILES: CNCC(=O)N[C@@H](Cc1ccc(OP(=O)(O)O)c(O)c1)C(=O)O] | MS (ESI); m/z 349.1 [M + H]+ |
| 13 | 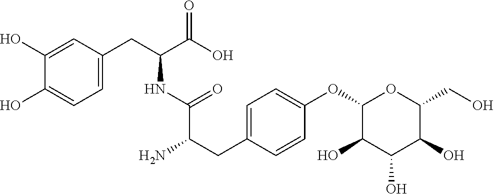 [SMILES: N[C@@H](Cc1ccc(O[C@@H]2O[C@H](CO)[C@@H](O)[C@H](O)[C@H]2O)cc1)C(=O)N[C@@H](Cc1ccc(O)c(O)c1)C(=O)O] | MS (ESI); m/z 523.0 [M + H]+ |
| 14 | 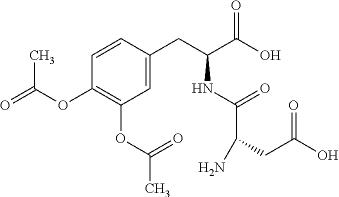 [SMILES: CC(=O)Oc1ccc(C[C@H](NC(=O)[C@@H](N)CC(=O)O)C(=O)O)cc1OC(C)=O] | MS (ESI); m/z 397.1 [M + H]+ |
| 15 | 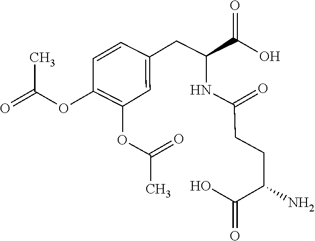 [SMILES: CC(=O)Oc1ccc(C[C@H](NC(=O)CC[C@H](N)C(=O)O)C(=O)O)cc1OC(C)=O] | MS (ESI); m/z 411.1 [M + H]+ |
| 16 | 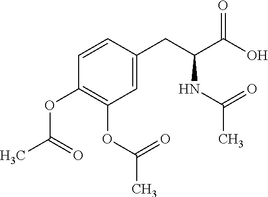 [SMILES: CC(=O)N[C@@H](Cc1ccc(OC(C)=O)c(OC(C)=O)c1)C(=O)O] | MS (ESI); m/z 324.1 [M + H]+ |
| 17 | 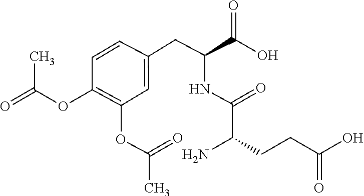 [SMILES: CC(=O)Oc1ccc(C[C@H](NC(=O)[C@@H](N)CCC(=O)O)C(=O)O)cc1OC(C)=O] | MS (ESI); m/z 411.1 [M + H]+ |
| 18 | 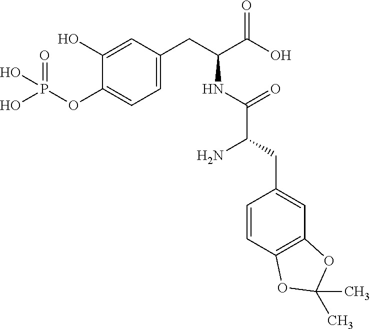 [SMILES: CC1(C)Oc2ccc(C[C@H](N)C(=O)N[C@@H](Cc3ccc(OP(=O)(O)O)c(O)c3)C(=O)O)cc2O1] | MS (ESI); m/z 495.0 [M − H]− |
| 19 | 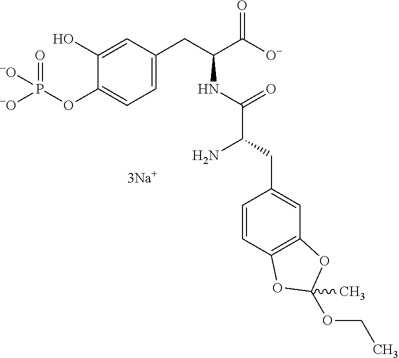 | MS (ESI); m/z 527.2 [M + H]+ |
| 101 | 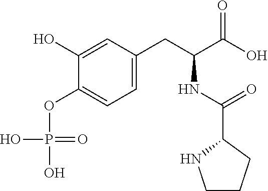 [SMILES: O=C(O)[C@H](Cc1ccc(OP(=O)(O)O)c(O)c1)NC(=O)[C@@H]1CCCN1] | MS(ESI);m/z 375.0[M + H]+ |
| 102 | 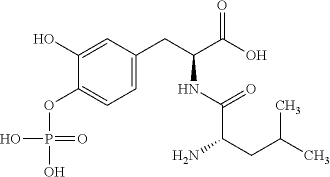 [SMILES: CC(C)C[C@H](N)C(=O)N[C@@H](Cc1ccc(OP(=O)(O)O)c(O)c1)C(=O)O] | MS(ESI);m/z 391.1[M + H]+ |
| 103 | 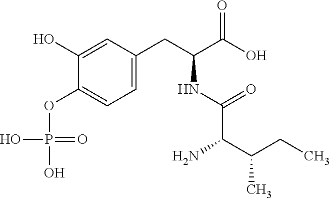 [SMILES: CC[C@H](C)[C@H](N)C(=O)N[C@@H](Cc1ccc(OP(=O)(O)O)c(O)c1)C(=O)O] | MS(ESI);m/z 391.1[M + H]+ |
| 104 | 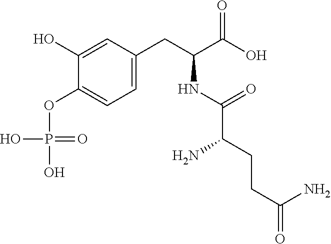 [SMILES: NC(=O)CC[C@H](N)C(=O)N[C@@H](Cc1ccc(OP(=O)(O)O)c(O)c1)C(=O)O] | MS(ESI);m/z 406.1[M + H]+ |
| 105 | 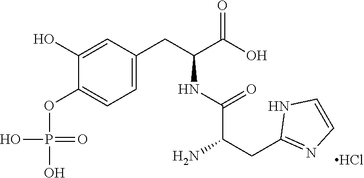 [SMILES: N[C@@H](Cc1ncc[nH]1)C(=O)N[C@@H](Cc1ccc(OP(=O)(O)O)c(O)c1)C(=O)O ; Cl] | MS(ESI);m/z 415.1[M + H]+ |
| 106 | 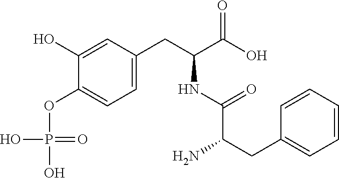 [SMILES: N[C@@H](Cc1ccccc1)C(=O)N[C@@H](Cc1ccc(OP(=O)(O)O)c(O)c1)C(=O)O] | MS(ESI);m/z 425.1[M + H]+ |
| 107 | 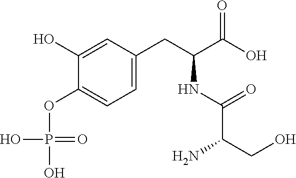 [SMILES: N[C@@H](CO)C(=O)N[C@@H](Cc1ccc(OP(=O)(O)O)c(O)c1)C(=O)O] | MS(ESI);m/z 365.0[M + H]+ |
| 108 | 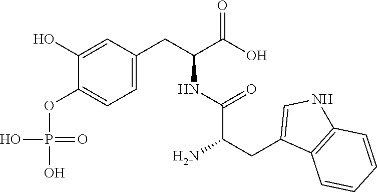 [SMILES: N[C@@H](Cc1c[nH]c2ccccc12)C(=O)N[C@@H](Cc1ccc(OP(=O)(O)O)c(O)c1)C(=O)O] | MS(ESI);m/z 464.1 [M + H]+ |
| 109 | 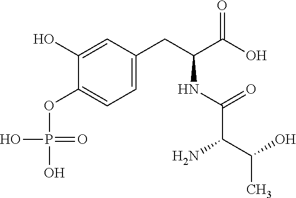 [SMILES: C[C@@H](O)[C@H](N)C(=O)N[C@@H](Cc1ccc(OP(=O)(O)O)c(O)c1)C(=O)O] | MS(ESI);m/z 379.0[M + H]+ |
| 110 | 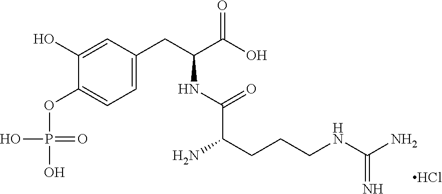 [SMILES: N=C(N)NCCC[C@H](N)C(=O)N[C@@H](Cc1ccc(OP(=O)(O)O)c(O)c1)C(=O)O ; Cl] | MS(ESI);m/z 434.1[M + H]+ |
| 111 | 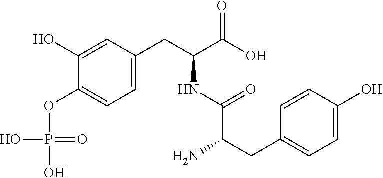 [SMILES: N[C@@H](Cc1ccc(O)cc1)C(=O)N[C@@H](Cc1ccc(OP(=O)(O)O)c(O)c1)C(=O)O] | MS(ESI);m/z 441.1 [M + H]+ |
| 112 | 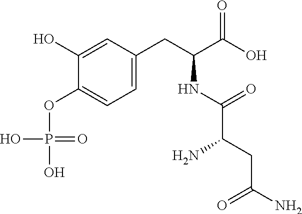 [SMILES: NC(=O)C[C@H](N)C(=O)N[C@@H](Cc1ccc(OP(=O)(O)O)c(O)c1)C(=O)O] | MS(ESI);m/z 392.0[M + H]+ |

### Example 20: Production of (2S)-2-[[(2S)-2-amino-5-carbamimidamide pentanoyl]amino]-3-(3,4-dihydroxyphenyl) propanoic acid; hydrochloride

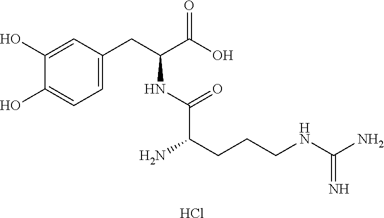 [SMILES: N=C(N)NCCC[C@H](N)C(=O)N[C@@H](Cc1ccc(O)c(O)c1)C(=O)O ; Cl]

(2S)-3-[3,4-bis(phenylmethoxy)phenyl]-2-[[(2S)-5-[(N-Nitrocarbamimideyl)amino]-2-(phenylmethoxycarbonylamino)pentanoyl]amino] benzyl propanoate (257 mg) was dissolved in a mixed solvent of tetrahydrofuran (2 mL), 2-propanol (3 mL) and 2M hydrochloric acid (0.80 mL), and palladium/carbon (hydrous) (341 mg) was added, and the mixture was stirred under a hydrogen atmosphere at room temperature for 24 hours. 2-propanol (4 mL) and water (6 mL) were added to the reaction mixture, and insoluble matter was removed using a membrane filter. The insoluble matter was washed with water (6 mL), 2-propanol (25 mL) was added, and the mixture was distilled under a reduced pressure. Diisopropyl ether was added to the residue, and a precipitated solid was collected by filtration and was dried under a reduced pressure, and thereby, the title compound (144 mg, yield: 100%) as a yellow-brown powder was obtained.

MS (ESI); m/z 354.2 [M+H]+

### Examples 21-28

The compounds shown in Table 3 below can be obtained in the same manner as above example.

TABLE 3

| Example | Structural formula |
|---|---|
| 21 | 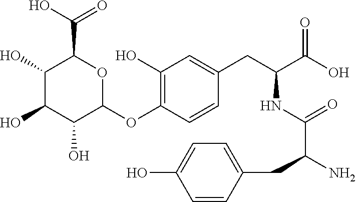 [SMILES: N[C@@H](Cc1ccc(O)cc1)C(=O)N[C@@H](Cc1ccc(OC2O[C@H](C(=O)O)[C@@H](O)[C@H](O)[C@H]2O)c(O)c1)C(=O)O] |
| 22 | 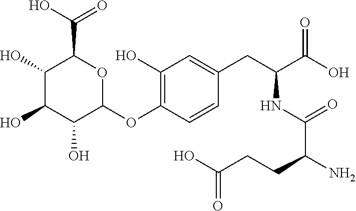 [SMILES: N[C@@H](CCC(=O)O)C(=O)N[C@@H](Cc1ccc(OC2O[C@H](C(=O)O)[C@@H](O)[C@H](O)[C@H]2O)c(O)c1)C(=O)O] |
| 23 | 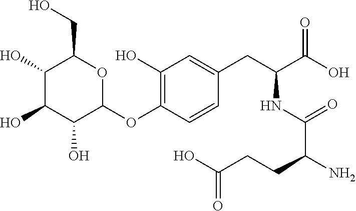 [SMILES: N[C@@H](CCC(=O)O)C(=O)N[C@@H](Cc1ccc(OC2O[C@H](CO)[C@@H](O)[C@H](O)[C@H]2O)c(O)c1)C(=O)O] |
| 24 | 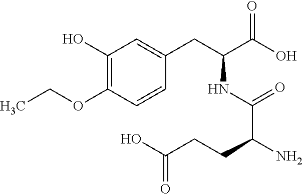 [SMILES: CCOc1ccc(C[C@H](NC(=O)[C@@H](N)CCC(=O)O)C(=O)O)cc1O] |
| 25 | 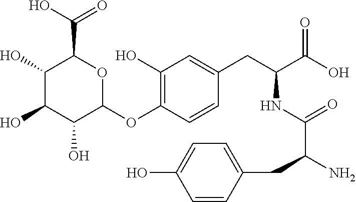 [SMILES: N[C@@H](Cc1ccc(O)cc1)C(=O)N[C@@H](Cc1ccc(OC2O[C@H](C(=O)O)[C@@H](O)[C@H](O)[C@H]2O)c(O)c1)C(=O)O] |
| 26 | 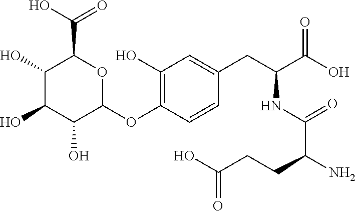 [SMILES: N[C@@H](CCC(=O)O)C(=O)N[C@@H](Cc1ccc(OC2O[C@H](C(=O)O)[C@@H](O)[C@H](O)[C@H]2O)c(O)c1)C(=O)O] |
| 27 | 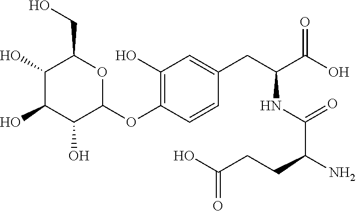 [SMILES: N[C@@H](CCC(=O)O)C(=O)N[C@@H](Cc1ccc(OC2O[C@H](CO)[C@@H](O)[C@H](O)[C@H]2O)c(O)c1)C(=O)O] |
| 28 | 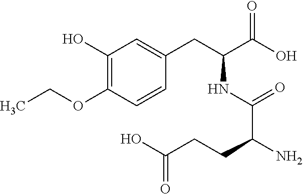 [SMILES: CCOc1ccc(C[C@H](NC(=O)[C@@H](N)CCC(=O)O)C(=O)O)cc1O] |

### Reference Example 1: Production of (2S)-3-[4-bis(phenylmethoxy)phosphoryloxy-3-phenylmethoxyphenyl]-2-[[(2S)-3-(2-ethoxy-2-methyl-1,3-benzodioxol-5)-1)-2-(phenylmethoxycarbonylamino)propanol]amino] proanoic acid

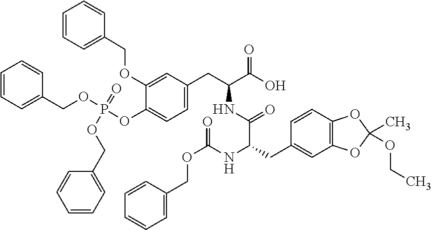 [SMILES: CCOC1(C)Oc2ccc(C[C@H](NC(=O)OCc3ccccc3)C(=O)N[C@@H](Cc3ccc(OP(=O)(OCc4ccccc4)OCc4ccccc4)c(OCc4ccccc4)c3)C(=O)O)cc2O1]

(2S)-3-[4-bis(phenylmethoxy)phosphoryloxy-3-phenylmethoxyphenyl]-2-[[(2S)-3-(2-ethoxy-2-methyl-1,3-benzodioxol-5-yl)-2-(phenylmethoxycarbonylamino)propanoyl]amino]benzyl propanoate (208 mg) was dissolved in a mixed solvent of water (0.41 mL) and tetrahydrofuran (1.0 mL), and lithium hydroxide monohydrate (9.4 mg) was added under an ice bath, and then, the mixture was stirred at room temperature for 2 hours. After adding ethyl acetate to the reaction mixture, a saturated aqueous citric acid solution was added until the pH of the mixture reached 6, and extraction with ethyl acetate was performed. An organic layer was dried over magnesium sulfate and insoluble matter was filtered, and then, the solvent was distilled away under a reduced pressure, and the residue was purified by silica gel column chromatography (solvent: hexane/(ethyl acetate)=70/30-0/100, (ethyl acetate)/methanol=100/0-85/15), and thereby, the title compound (179 mg, yield: 94%) as a colorless viscous substance was obtained.

MS (ESI); m/z 929.3 [M−H]−

### Reference Example 2: Production of (2S)-3-[4-bis(phenylmethoxy)phosphoryloxy-3-phenylmethoxyphenyl]-2-[[(2S)-2-(phenylmethoxycarbonylamino)propanoyl]amino] benzyl propanoate

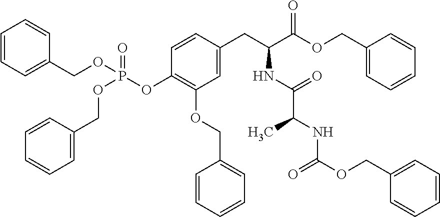 [SMILES: C[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1]

1H-tetrazole (72 mg) and dibenzyl N,N-diisopropylphosphoroamidite (0.38 mL) were added under ice-cooling to a mixture of (2S)-3-(4-hydroxy-3-phenylmethoxyphenyl)-2-[[(2S)-2-(phenylmethoxycarbonylamino)propanoyl]amino] benzyl propanoate (387 mg), dichloromethane (4 mL) and acetonitrile (1.6 mL), and the mixture was stirred at room temperature for 3 hours. The reaction mixture was ice-cooled, a tert-butyl hydroperoxide decane solution (5.5 M) (0.18 mL) was added, and the mixture was stirred at room temperature for 1 hour. The solvent of the reaction mixture was distilled away under reduced pressure, and then, toluene was added, and insoluble matter was removed with Phase-separator®. The filtrate was purified by silica gel column chromatography (solvent: hexane/(ethyl acetate)=67/33-40/60), and thereby, the title compound (551 mg, yield: 90%) as a colorless viscous substance was obtained.

MS (ESI); m/z 843.7 [M+H]+

### Reference Examples 3-16

The corresponding starting compounds were respectively treated in the same manner as in Reference Example 2 to obtain the compounds shown in Table 4 below.

TABLE 4

| Reference | | Physical property |
|---|---|---|
| Example | Structural formula | values and the like |
| 3 | 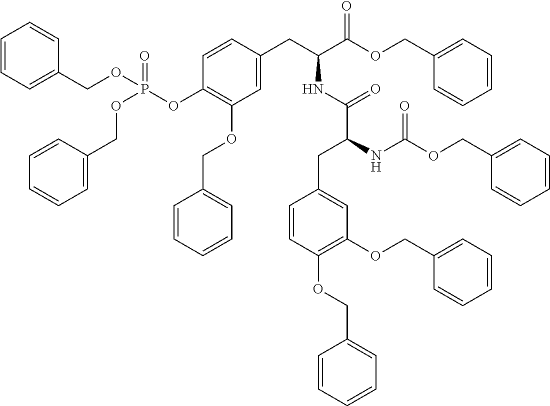 [SMILES: O=C(N[C@@H](Cc1ccc(OCc2ccccc2)c(OCc2ccccc2)c1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS (ESI); m/z 1148.6 [M + H + NH2]+ |
| 4 | 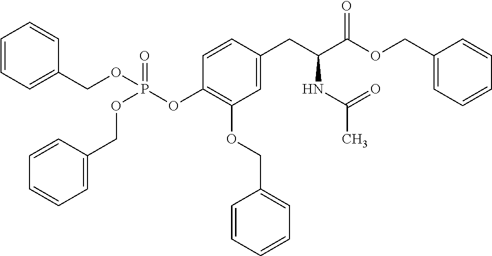 [SMILES: CC(=O)N[C@@H](Cc1ccc(OP(=O)(OCc2ccccc2)OCc2ccccc2)c(OCc2ccccc2)c1)C(=O)OCc1ccccc1] | MS (ESI); m/z 680.3 [M + H]+ |
| 5 | 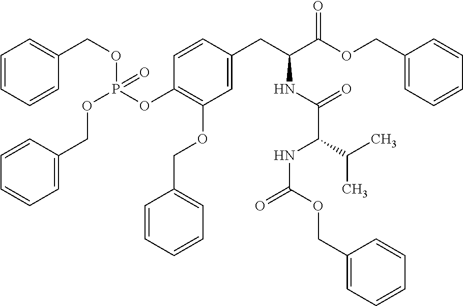 | MS (ESI); m/z 915.7 [M − H + HCO2H]− |
| 6 | 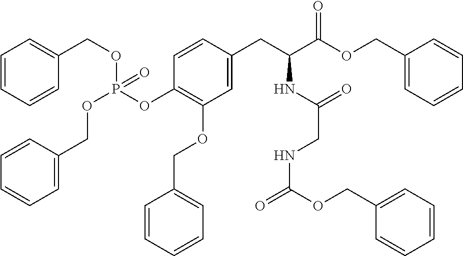 [SMILES: O=C(CNC(=O)OCc1ccccc1)N[C@@H](Cc1ccc(OP(=O)(OCc2ccccc2)OCc2ccccc2)c(OCc2ccccc2)c1)C(=O)OCc1ccccc1] | MS (ESI); m/z 873.6 [M − H + HCO2H]− |
| 7 | 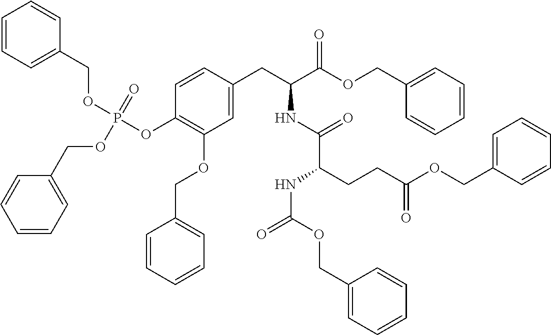 [SMILES: O=C(CC[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS (ESI); m/z 989.7 [M − H]− |
| 8 | 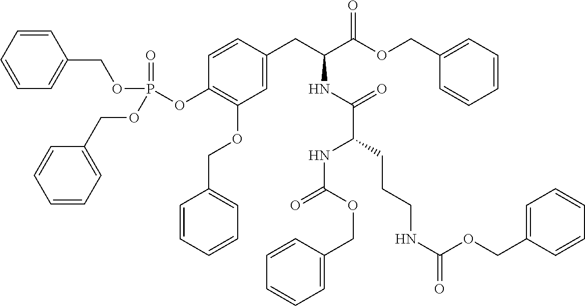 [SMILES: O=C(NCCC[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS (ESI); m/z 1018.4 [M − H]− |
| 9 | 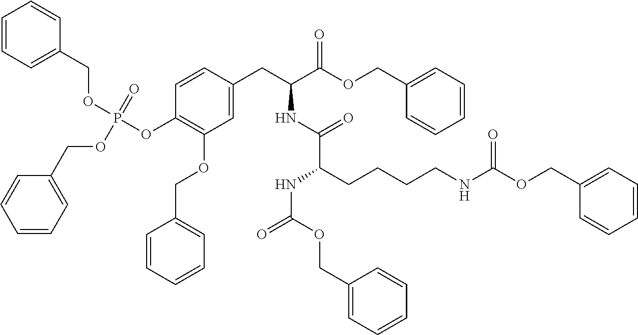 [SMILES: O=C(NCCCC[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS (ESI); m/z 1034.8 [M + H]+ |
| 10 | 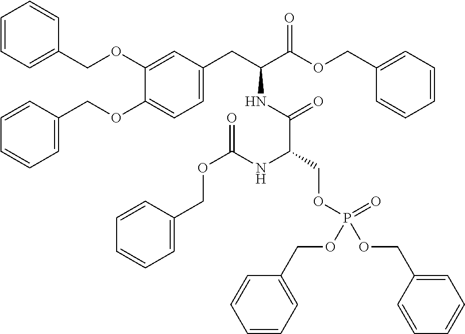 [SMILES: O=C(N[C@@H](COP(=O)(OCc1ccccc1)OCc1ccccc1)C(=O)N[C@@H](Cc1ccc(OCc2ccccc2)c(OCc2ccccc2)c1)C(=O)OCc1ccccc1)OCc1ccccc1] | MS (ESI); m/z 949.2 [M + H]+ |
| 11 | 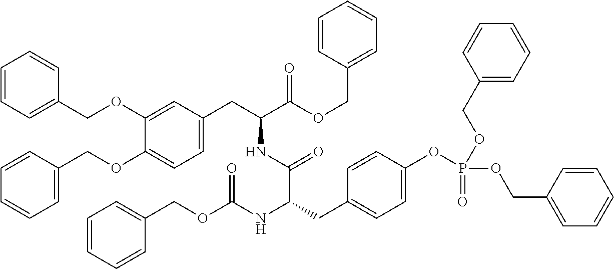 [SMILES: O=C(N[C@@H](Cc1ccc(OP(=O)(OCc2ccccc2)OCc2ccccc2)cc1)C(=O)N[C@@H](Cc1ccc(OCc2ccccc2)c(OCc2ccccc2)c1)C(=O)OCc1ccccc1)OCc1ccccc1] | MS (ESI); m/z 1025.7 [M + H]+ |
| 12 | 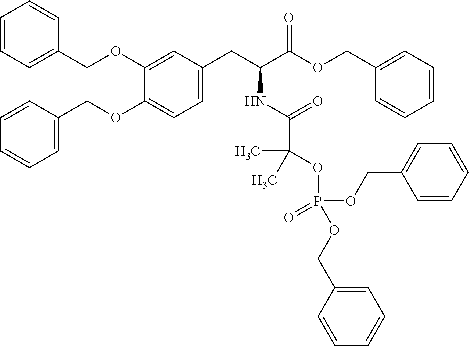 [SMILES: CC(C)(OP(=O)(OCc1ccccc1)OCc1ccccc1)C(=O)N[C@@H](Cc1ccc(OCc2ccccc2)c(OCc2ccccc2)c1)C(=O)OCc1ccccc1] | MS (ESI); m/z 814.4 [M + H]+ |
| 13 | 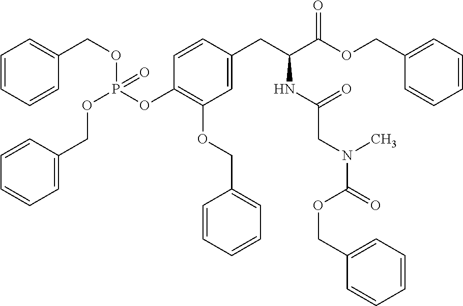 [SMILES: CN(CC(=O)N[C@@H](Cc1ccc(OP(=O)(OCc2ccccc2)OCc2ccccc2)c(OCc2ccccc2)c1)C(=O)OCc1ccccc1)C(=O)OCc1ccccc1] | MS (ESI); m/z 843.7 [M + H]+ |
| 14 | 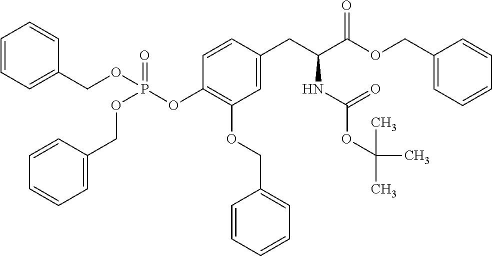 [SMILES: CC(C)(C)OC(=O)N[C@@H](Cc1ccc(OP(=O)(OCc2ccccc2)OCc2ccccc2)c(OCc2ccccc2)c1)C(=O)OCc1ccccc1] | MS (ESI); m/z 638.6 [M + H − Boc]+ |
| 15 | 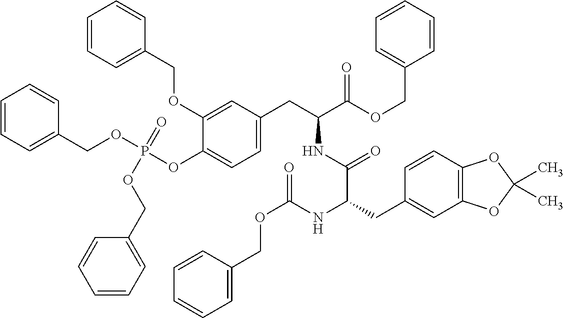 [SMILES: CC1(C)Oc2ccc(C[C@H](NC(=O)OCc3ccccc3)C(=O)N[C@@H](Cc3ccc(OP(=O)(OCc4ccccc4)OCc4ccccc4)c(OCc4ccccc4)c3)C(=O)OCc3ccccc3)cc2O1] | MS (ESI); m/z 989.5 [M − H]− |
| 16 | 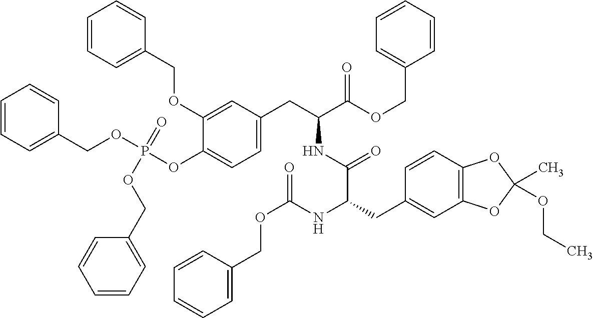 [SMILES: CCOC1(C)Oc2ccc(C[C@H](NC(=O)OCc3ccccc3)C(=O)N[C@@H](Cc3ccc(OP(=O)(OCc4ccccc4)OCc4ccccc4)c(OCc4ccccc4)c3)C(=O)OCc3ccccc3)cc2O1] | MS (ESI); m/z 1019.3 [M − H]− |

### Reference Example 101

### Production of benzyl (2S)-3-[4-bis(phenylmethoxy)phosphoryloxy-3-phenylmethoxyphenyl]-2-[[(2S)-4-methyl-2-(phenylmethoxycarbonylamino)pentanoyl]amino] propanoato

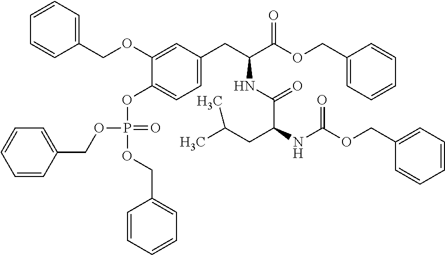 [SMILES: CC(C)C[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1]

1,8-diazabicyclo[5.4.0]undec-7-ene (223 uL) and tetrabenzyl diphosphate (805 mg) were added to a mixture of benzyl (2S)-3-(4-hydroxy-3-phenylmethoxyphenyl)-2-[[(2S)-4-methyl-2-(phenylmethoxycarbonylamino)pentanoyl]amino]propanoato (623 mg) and acetonitrile (5 mL) under ice cooling, and the temperature was gradually raised to room temperature, and the mixture was stirred for 21 hours. 1,8-diazabicyclo[5.4.0]undec-7-ene (74 uL) and tetrabenzyl diphosphate (266 mg) were added to the mixture, and the mixture was stirred for 3.5 hours. A saturated aqueous sodium hydrogen carbonate solution was added to the reaction mixture, and extraction with ethyl acetate was performed. The organic layer was dried over magnesium sulfate, and the solvent was removed under reduced pressure. The obtained residue was purified by silica gel column chromatography (solvent: hexane/(ethyl acetate)=67/33 to 40/60), and thereby, the title compound (702 mg, 80% yield) as a colorless mucus was obtained.

MS (ESI); m/z 885.3 [M+H]+

### Reference Examples 102 to 112

The corresponding starting compounds were respectively treated in the same manner as in Reference Example 101 to obtain the compounds shown in Table 8-5 below. Some of the compounds shown in Table 8-5 below can be obtained in the same manner as above example.

TABLE 8-5

| Reference | | Physical property |
|---|---|---|
| Example | Structural formula | values and the like |
| 102 | 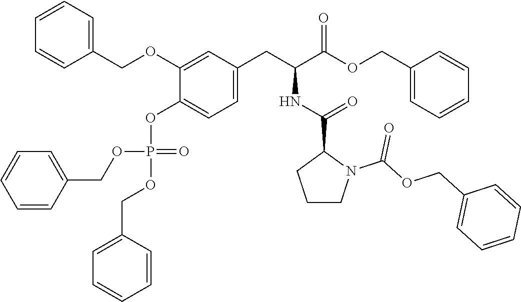 [SMILES: O=C(OCc1ccccc1)[C@H](Cc1ccc(OP(=O)(OCc2ccccc2)OCc2ccccc2)c(OCc2ccccc2)c1)NC(=O)[C@@H]1CCCN1C(=O)OCc1ccccc1] | MS(ESI);m/z 867.2[M − H]− |
| 103 | 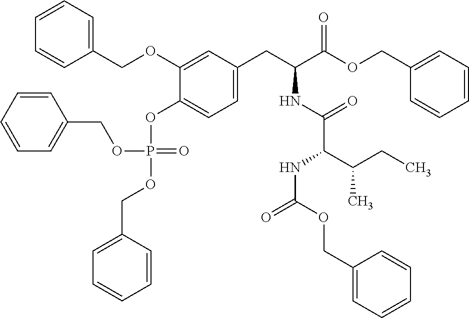 [SMILES: CC[C@H](C)[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1] | MS(ESI);m/z 885.2[M + H]+ |
| 104 | 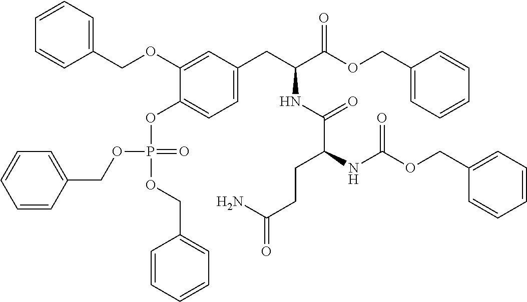 [SMILES: NC(=O)CC[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1] | MS(ESI);m/z 900.2[M + H]+ |
| 105 | 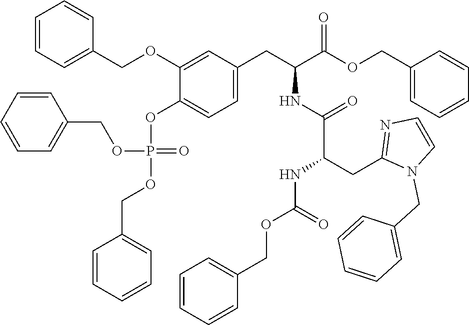 [SMILES: O=C(N[C@@H](Cc1nccn1Cc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS(ESI);m/z 999.2[M + H]+ |
| 106 | 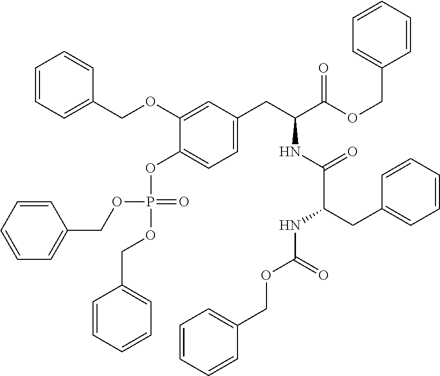 [SMILES: O=C(N[C@@H](Cc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS(ESI);m/z 919.2[M + H]+ |
| 107 | 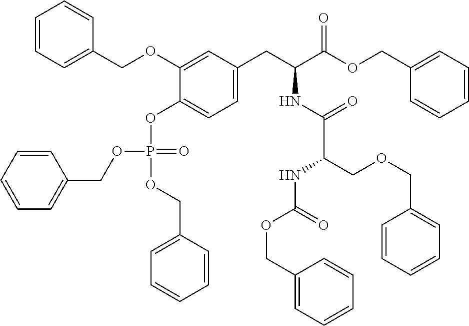 [SMILES: O=C(N[C@@H](COCc1ccccc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS(ESI);m/z 949.2[M + H]+ |
| 108 | 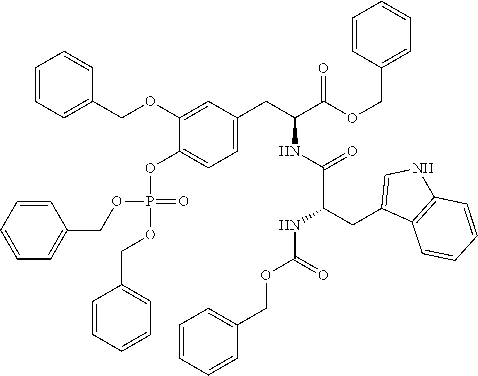 [SMILES: O=C(N[C@@H](Cc1c[nH]c2ccccc12)C(=O)N[C@@H](Cc1ccc(OP(=O)(OCc2ccccc2)OCc2ccccc2)c(OCc2ccccc2)c1)C(=O)OCc1ccccc1)OCc1ccccc1] | |
| 109 | 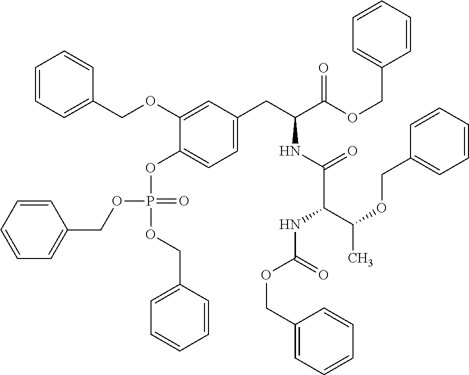 [SMILES: C[C@@H](OCc1ccccc1)[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1] | MS(ESI);m/z 963.3[M + H]+ |
| 110 | 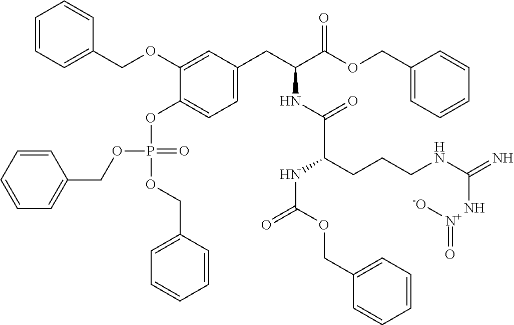 | MS(ESI);m/z 973.2[M + H]+ |
| 111 | 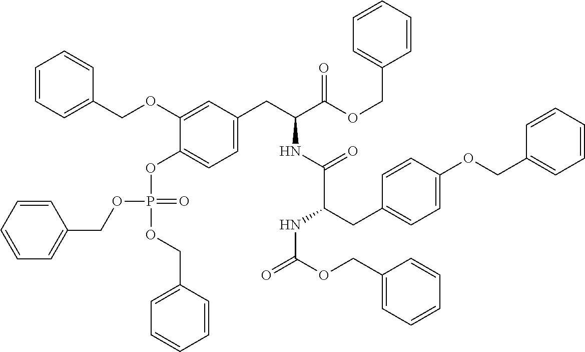 [SMILES: O=C(N[C@@H](Cc1ccc(OCc2ccccc2)cc1)C(=O)N[C@@H](Cc1ccc(OP(=O)(OCc2ccccc2)OCc2ccccc2)c(OCc2ccccc2)c1)C(=O)OCc1ccccc1)OCc1ccccc1] | MS(ESI);m/z 1025.3[M + H]+ |
| 112 | 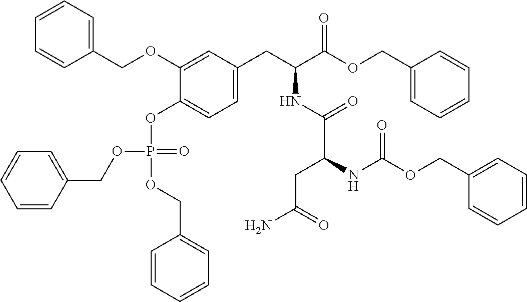 [SMILES: NC(=O)C[C@H](NC(=O)OCc1ccccc1)C(=O)N[C@@H](Cc1ccc(OP(=O)(OCc2ccccc2)OCc2ccccc2)c(OCc2ccccc2)c1)C(=O)OCc1ccccc1] | MS(ESI);m/z 886.2[M + H]+ |

### Reference Example 17: Production of (2S)-3-(4-hydroxy-3-phenylmethoxyphenyl)-2-[[(2S)-2-(phenylmethoxycarbonylamino)propanoyl]amino] benzyl propanoate

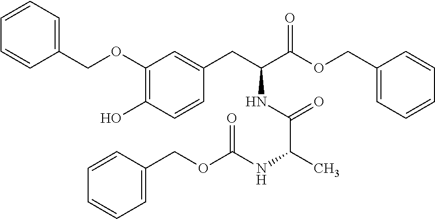 [SMILES: C[C@H](NC(=O)OCc1ccccc1)C(=O)N[C@@H](Cc1ccc(O)c(OCc2ccccc2)c1)C(=O)OCc1ccccc1]

(2S)-2-(benzyloxycarbonylamino) propanoic acid (1.02 g), 1-hydroxy-7-azabenzotriazole (HOAt) (712 mg), 1-ethyl-3-(3-dimethylaminopropyl) carbodiimide hydrochloride (WSCI) (1.03 g), and N,N-diisopropylethylamine (0.750 mL) were added to a mixture of (2S)-2-amino-3-(4-hydroxy-3-phenylmethoxyphenyl) benzyl propanoate; hydrochloride (1.87 g) and N,N-dimethylformamide (18 mL), and the mixture was stirred at room temperature for 18 hours. A saturated aqueous sodium hydrogen carbonate solution and water were added to the reaction mixture, and extraction with ethyl acetate was performed. An organic layer is washed with water and a saturated solution of sodium chloride and was dried over magnesium sulfate. Insoluble matter was filtered, and the solvent was distilled away under a reduced pressure. The residue was purified by silica gel column chromatography (solvent: hexane/(ethyl acetate)=67/33-45/55), and thereby, the title compound (2.56 g, yield: 99%) as a white powder was obtained.

MS (ESI); m/z 583.6 [M+H]+

### Reference Examples 18-34

The corresponding starting compounds were respectively treated in the same manner as in Reference Example 17 to obtain the compounds shown in Table 5 below.

TABLE 5

| Reference | | Physical property |
|---|---|---|
| Example | Structural formula | values and the like |
| 18 | 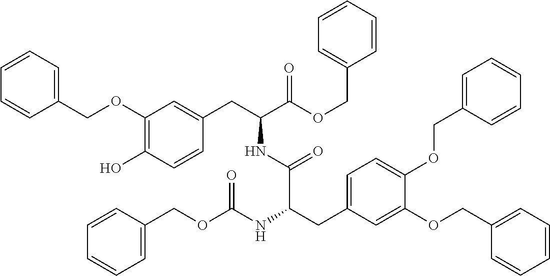 [SMILES: O=C(N[C@@H](Cc1ccc(OCc2ccccc2)c(OCc2ccccc2)c1)C(=O)N[C@@H](Cc1ccc(O)c(OCc2ccccc2)c1)C(=O)OCc1ccccc1)OCc1ccccc1] | MS (ESI); m/z 871.8 [M + H]+ |
| 19 | 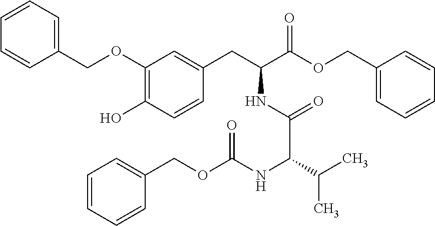 [SMILES: CC(C)[C@H](NC(=O)OCc1ccccc1)C(=O)N[C@@H](Cc1ccc(O)c(OCc2ccccc2)c1)C(=O)OCc1ccccc1] | MS (ESI); m/z 611.6 [M + H]+ |
| 20 | 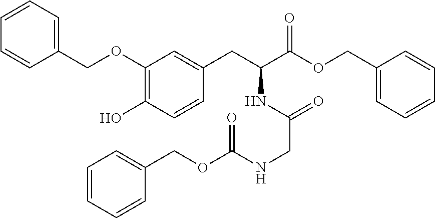 [SMILES: O=C(CNC(=O)OCc1ccccc1)N[C@@H](Cc1ccc(O)c(OCc2ccccc2)c1)C(=O)OCc1ccccc1] | MS (ESI); m/z 569.5 [M + H]+ |
| 21 | 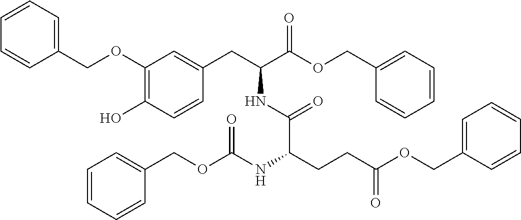 [SMILES: O=C(CC[C@H](NC(=O)OCc1ccccc1)C(=O)N[C@@H](Cc1ccc(O)c(OCc2ccccc2)c1)C(=O)OCc1ccccc1)OCc1ccccc1] | MS (ESI); m/z 731.4 [M + H]+ |
| 22 | 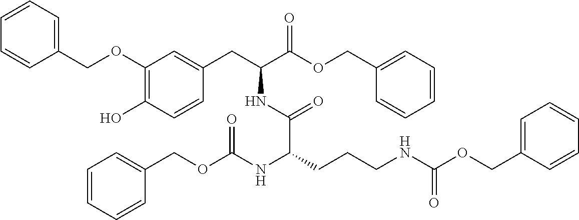 [SMILES: O=C(NCCC[C@H](NC(=O)OCc1ccccc1)C(=O)N[C@@H](Cc1ccc(O)c(OCc2ccccc2)c1)C(=O)OCc1ccccc1)OCc1ccccc1] | MS (ESI); m/z 760.2 [M + H]+ |
| 23 | 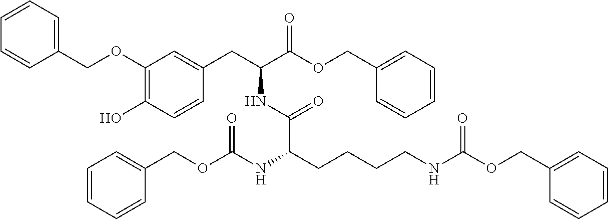 [SMILES: O=C(NCCCC[C@H](NC(=O)OCc1ccccc1)C(=O)N[C@@H](Cc1ccc(O)c(OCc2ccccc2)c1)C(=O)OCc1ccccc1)OCc1ccccc1] | MS (ESI); m/z 774.7 [M + H]+ |
| 24 | 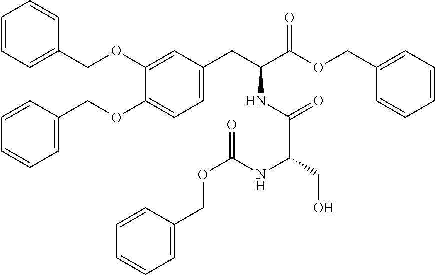 [SMILES: O=C(N[C@@H](CO)C(=O)N[C@@H](Cc1ccc(OCc2ccccc2)c(OCc2ccccc2)c1)C(=O)OCc1ccccc1)OCc1ccccc1] | MS (ESI); m/z 689.5 [M + H]+ |
| 25 | 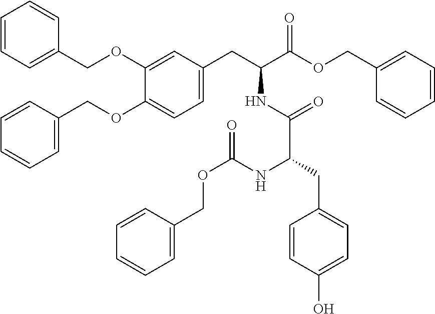 [SMILES: O=C(N[C@@H](Cc1ccc(O)cc1)C(=O)N[C@@H](Cc1ccc(OCc2ccccc2)c(OCc2ccccc2)c1)C(=O)OCc1ccccc1)OCc1ccccc1] | MS (ESI); m/z 765.3 [M + H]+ |
| 26 | 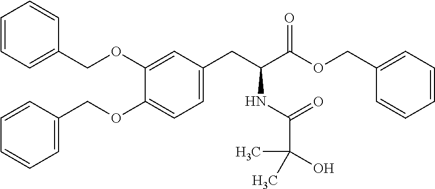 [SMILES: CC(C)(O)C(=O)N[C@@H](Cc1ccc(OCc2ccccc2)c(OCc2ccccc2)c1)C(=O)OCc1ccccc1] | MS (ESI); m/z 554.2 [M + H]+ |
| 27 | 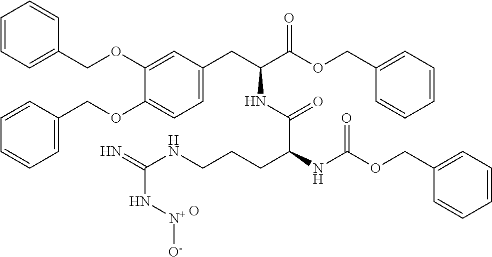 | MS (ESI); m/z 803.7 [M + H]+ |
| 28 | 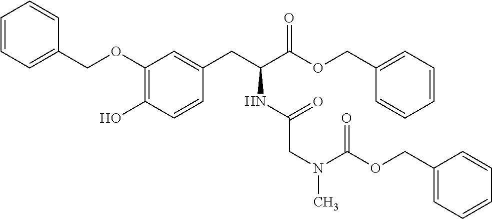 [SMILES: CN(CC(=O)N[C@@H](Cc1ccc(O)c(OCc2ccccc2)c1)C(=O)OCc1ccccc1)C(=O)OCc1ccccc1] | MS (ESI); m/z 581.3 [M − H]− |
| 29 | 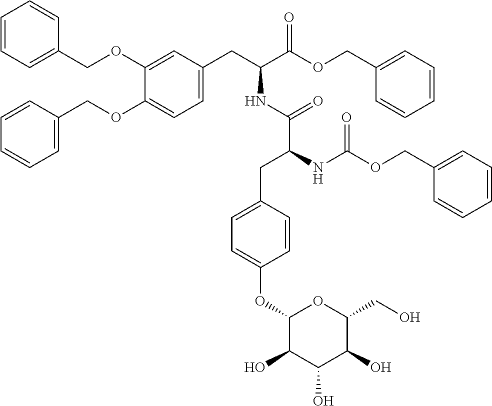 [SMILES: O=C(N[C@@H](Cc1ccc(O[C@@H]2O[C@H](CO)[C@@H](O)[C@H](O)[C@H]2O)cc1)C(=O)N[C@@H](Cc1ccc(OCc2ccccc2)c(OCc2ccccc2)c1)C(=O)OCc1ccccc1)OCc1ccccc1] | MS (ESI); m/z 927.7 [M + H]+ |
| 30 | 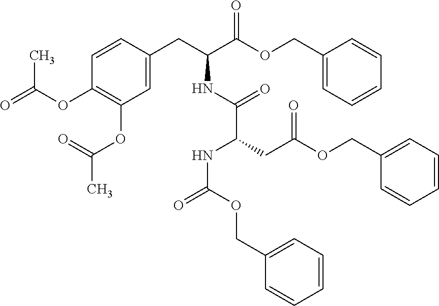 [SMILES: CC(=O)Oc1ccc(C[C@H](NC(=O)[C@H](CC(=O)OCc2ccccc2)NC(=O)OCc2ccccc2)C(=O)OCc2ccccc2)cc1OC(C)=O] | MS (ESI); m/z 711.4 [M + H]+ |
| 31 | 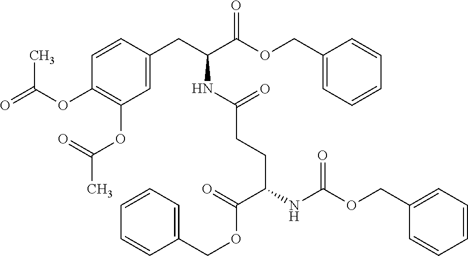 [SMILES: CC(=O)Oc1ccc(C[C@H](NC(=O)CC[C@H](NC(=O)OCc2ccccc2)C(=O)OCc2ccccc2)C(=O)OCc2ccccc2)cc1OC(C)=O] | MS (ESI); m/z 725.5 [M + H]+ |
| 32 | 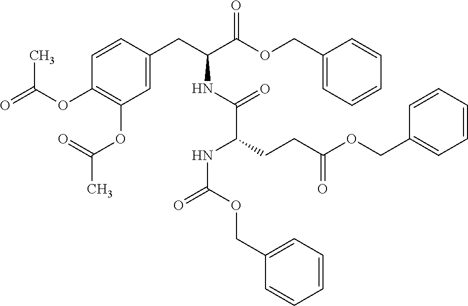 [SMILES: CC(=O)Oc1ccc(C[C@H](NC(=O)[C@H](CCC(=O)OCc2ccccc2)NC(=O)OCc2ccccc2)C(=O)OCc2ccccc2)cc1OC(C)=O] | MS (ESI); m/z 725.4 [M + H]+ |
| 33 | 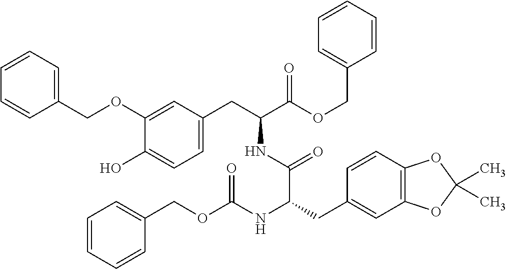 [SMILES: CC1(C)Oc2ccc(C[C@H](NC(=O)OCc3ccccc3)C(=O)N[C@@H](Cc3ccc(O)c(OCc4ccccc4)c3)C(=O)OCc3ccccc3)cc2O1] | MS (ESI); m/z 729.3 [M − H]− |
| 34 | 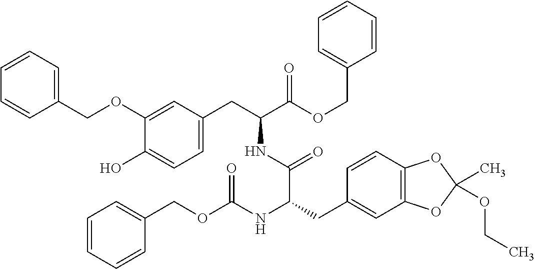 [SMILES: CCOC1(C)Oc2ccc(C[C@H](NC(=O)OCc3ccccc3)C(=O)N[C@@H](Cc3ccc(O)c(OCc4ccccc4)c3)C(=O)OCc3ccccc3)cc2O1] | MS (ESI); m/z 761.0 [M + H]+ |
| 113 | 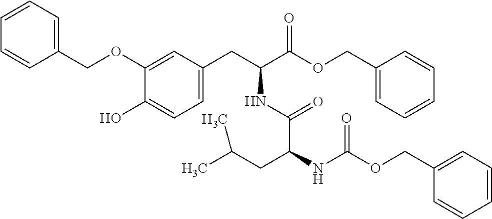 [SMILES: CC(C)C[C@H](NC(=O)OCc1ccccc1)C(=O)N[C@@H](Cc1ccc(O)c(OCc2ccccc2)c1)C(=O)OCc1ccccc1] | MS(ESI);m/z 625.3[M + H]+ |
| 114 | 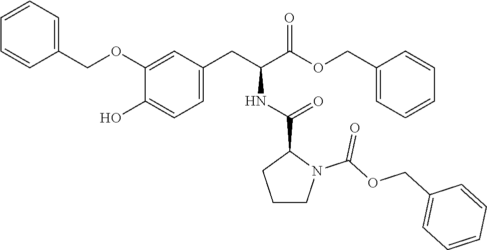 [SMILES: O=C(OCc1ccccc1)[C@H](Cc1ccc(O)c(OCc2ccccc2)c1)NC(=O)[C@@H]1CCCN1C(=O)OCc1ccccc1] | MS(ESI);m/z 609.2[M + H]+ |
| 115 | 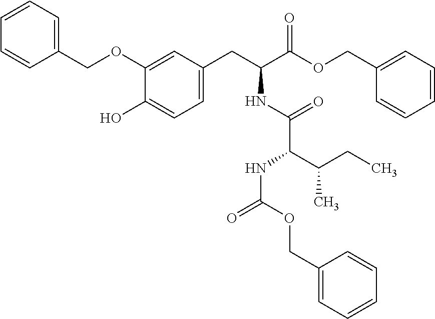 [SMILES: CC[C@H](C)[C@H](NC(=O)OCc1ccccc1)C(=O)N[C@@H](Cc1ccc(O)c(OCc2ccccc2)c1)C(=O)OCc1ccccc1] | MS(ESI);m/z 624.9[M + H]+ |
| 116 | 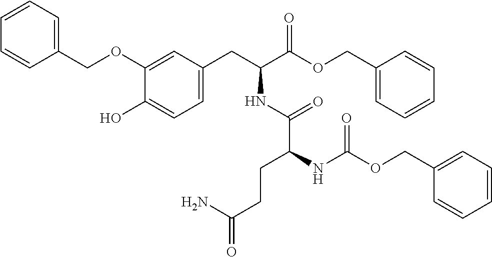 [SMILES: NC(=O)CC[C@H](NC(=O)OCc1ccccc1)C(=O)N[C@@H](Cc1ccc(O)c(OCc2ccccc2)c1)C(=O)OCc1ccccc1] | MS(ESI);m/z 640.2[M + H]+ |
| 117 | 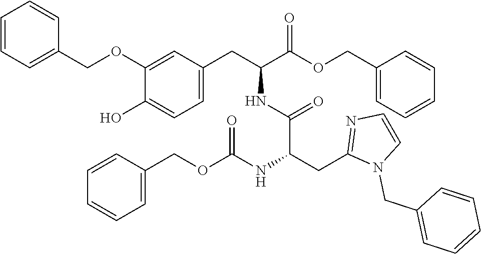 [SMILES: O=C(N[C@@H](Cc1nccn1Cc1ccccc1)C(=O)N[C@@H](Cc1ccc(O)c(OCc2ccccc2)c1)C(=O)OCc1ccccc1)OCc1ccccc1] | MS(ESI);m/z 739.3[M + H]+ |
| 118 | 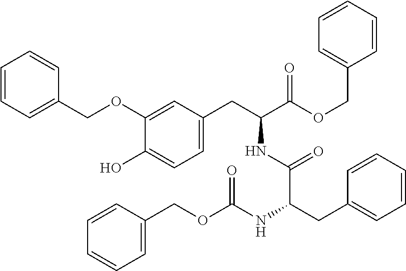 [SMILES: O=C(N[C@@H](Cc1ccccc1)C(=O)N[C@@H](Cc1ccc(O)c(OCc2ccccc2)c1)C(=O)OCc1ccccc1)OCc1ccccc1] | MS(ESI);m/z 659.2[M + H]+ |
| 119 | 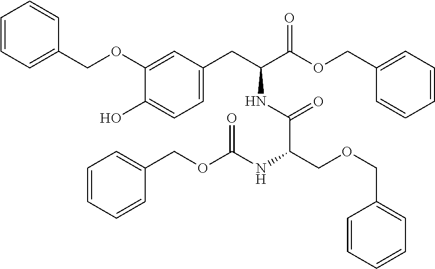 [SMILES: O=C(N[C@@H](COCc1ccccc1)C(=O)N[C@@H](Cc1ccc(O)c(OCc2ccccc2)c1)C(=O)OCc1ccccc1)OCc1ccccc1] | MS(ESI);m/z 689.3[M + H]+ |
| 120 | 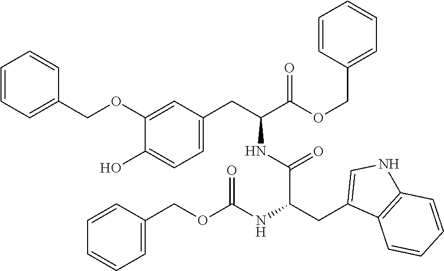 [SMILES: O=C(N[C@@H](Cc1c[nH]c2ccccc12)C(=O)N[C@@H](Cc1ccc(O)c(OCc2ccccc2)c1)C(=O)OCc1ccccc1)OCc1ccccc1] | MS(ESI);m/z 698.2[M + H]+ |
| 121 | 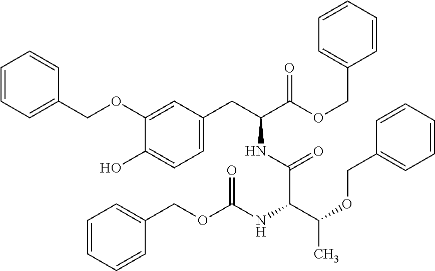 [SMILES: C[C@@H](OCc1ccccc1)[C@H](NC(=O)OCc1ccccc1)C(=O)N[C@@H](Cc1ccc(O)c(OCc2ccccc2)c1)C(=O)OCc1ccccc1] | MS(ESI);m/z 703.3[M + H]+ |
| 122 | 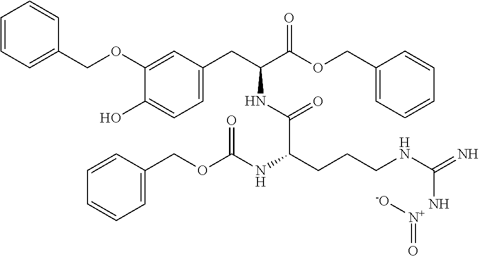 | MS(ESI);m/z 713.3[M + H]+ |
| 123 | 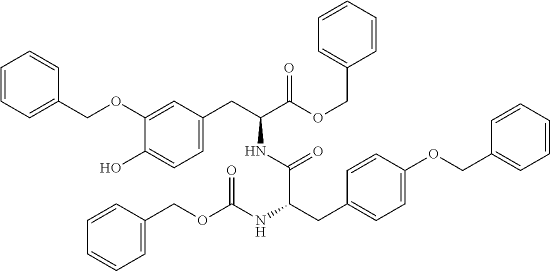 [SMILES: O=C(N[C@@H](Cc1ccc(OCc2ccccc2)cc1)C(=O)N[C@@H](Cc1ccc(O)c(OCc2ccccc2)c1)C(=O)OCc1ccccc1)OCc1ccccc1] | MS(ESI);m/z 765.3[M + H]+ |
| 124 | 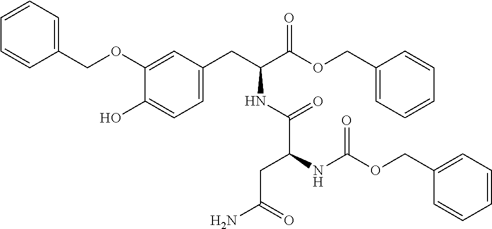 [SMILES: NC(=O)C[C@H](NC(=O)OCc1ccccc1)C(=O)N[C@@H](Cc1ccc(O)c(OCc2ccccc2)c1)C(=O)OCc1ccccc1] | MS(ESI);m/z 626.1[M + H]+ |

### Reference Example 35: Production of (2S)-2-acetamido-3-(3,4-diacetyloxyphenyl) benzyl propanoate

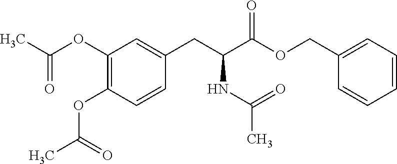 [SMILES: CC(=O)N[C@@H](Cc1ccc(OC(C)=O)c(OC(C)=O)c1)C(=O)OCc1ccccc1]

Acetic anhydride (0.291 mL) was added under ice-cooling to a mixture of (2S)-2-amino-3-(3,4-dihydroxyphenyl) benzyl propanoate; hydrochloride (253 mg) and pyridine (1 mL), and the mixture was stirred at room temperature for 18 hours. A saturated aqueous sodium hydrogen carbonate solution was added to the reaction mixture, and extraction with chloroform was performed. An organic layer was washed with a saturated solution of sodium chloride and was dried over sodium sulfate, and insoluble matter was filtered, and then, the solvent was distilled away under a reduced pressure. The obtained residue was purified by silica gel column chromatography (solvent: hexane/(ethyl acetate)=80/20-0/100), and thereby, the title compound (280 mg, yield: 72%) as a colorless viscous substance was obtained.

MS (ESI); m/z 414.2 [M+H]+

### Reference Examples 36 and 37

The corresponding starting compounds were respectively treated in the same manner as in Reference Example 35 to obtain the compounds shown in Table 6 below.

TABLE 6

| Reference | | Physical property values and the |
|---|---|---|
| Example | Structural formula | like |
| 36 | 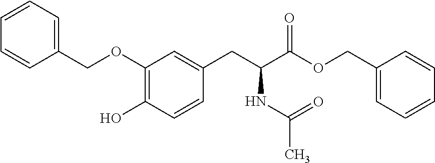 [SMILES: CC(=O)N[C@@H](Cc1ccc(O)c(OCc2ccccc2)c1)C(=O)OCc1ccccc1] | MS (ESI); m/z 420.5 [M + H]+ |
| 37 | 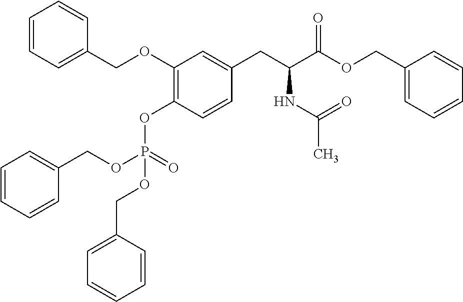 [SMILES: CC(=O)N[C@@H](Cc1ccc(OP(=O)(OCc2ccccc2)OCc2ccccc2)c(OCc2ccccc2)c1)C(=O)OCc1ccccc1] | MS (ESI); m/z 680.3 [M + H]+ |

### Reference Example 38: Production of (2S)-2-amino-3-[3,4-bis(phenylmethoxy)phenyl]benzyl propanoate; hydrochloride

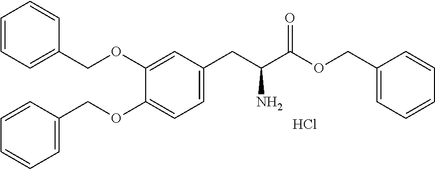 [SMILES: N[C@@H](Cc1ccc(OCc2ccccc2)c(OCc2ccccc2)c1)C(=O)OCc1ccccc1 ; Cl]

A 4M hydrogen chloride dioxane solution (43 mL) was added under ice cooling to (2S)-3-[3,4-bis(phenylmethoxy)phenyl]-2-[(2-methylpropan-2-yl)oxycarbonylamino] benzyl propanoate (13.2 g), and the mixture was stirred at room temperature for 3 hours. A 4M hydrogen chloride ethyl acetate solution (3 mL) was added, and the mixture was stirred for 2 hours. The solvent of the reaction mixture was distilled away under a reduced pressure. The residue was suspended in diisopropyl ether, and a precipitated solid was collected by filtration and was dried under a reduced pressure, and thereby, the title compound (10.4 mg, yield: 96%) as a white powder was obtained.

MS (ESI); m/z 468.3 [M+H]+

### Reference Example 39

The corresponding starting compounds were respectively treated in the same manner as in Reference Example 33 to obtain the compounds shown in Table 7 below.

TABLE 7

| Reference | | Physical property values and the |
|---|---|---|
| Example | Structural formula | like |
| 39 | 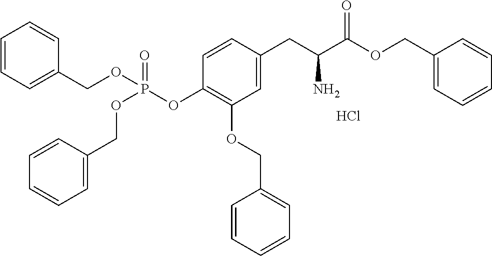 [SMILES: N[C@@H](Cc1ccc(OP(=O)(OCc2ccccc2)OCc2ccccc2)c(OCc2ccccc2)c1)C(=O)OCc1ccccc1 ; Cl] | MS (ESI); m/z 638.3 [M + H]+ |

### Reference Example 40: Production of (2S)-2-amino-3-(3-benzyloxy-4-hydroxyphenyl) benzyl propanoate; hydrochloride

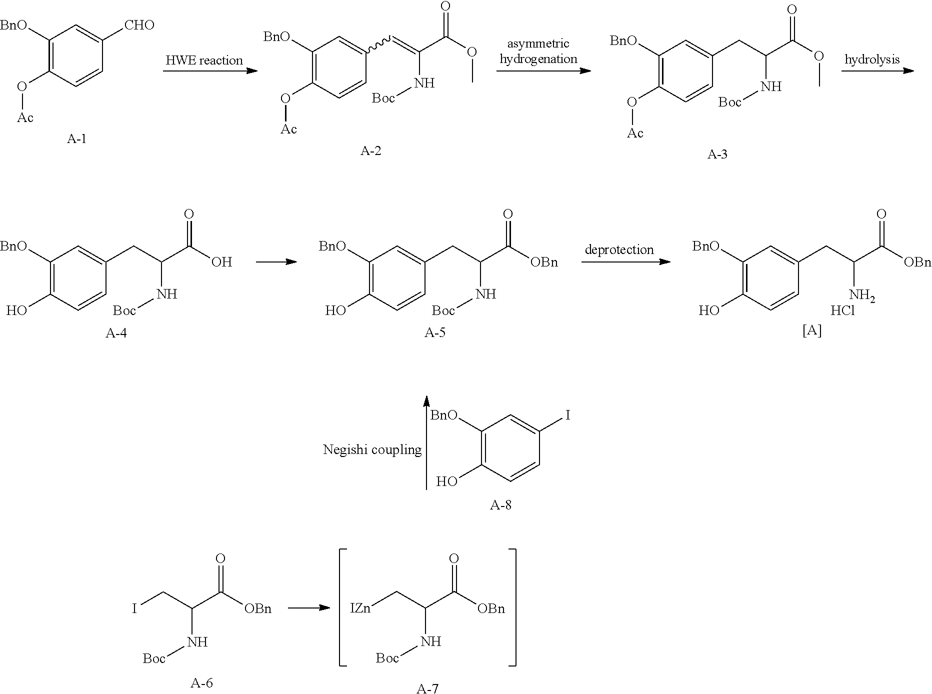

(1) N-(tert-butoxycarbonyl)-phosphonoglycine trimethyl ester (10 g) and 1,1,3,3-tetramethylguanidine (5 mL) were added under ice-cooling to a mixture of the compound A-1 (8.31 g) and dichloromethane (90 mL), and the mixture was stirred at room temperature for 24 hours. A saturated aqueous sodium hydrogen carbonate solution and water were added to the reaction mixture, and extraction with ethyl acetate was performed. An organic layer was passed through silica gel column chromatography (solvent: ethyl acetate), and the solvent of the filtrate was distilled away under a reduced pressure. The residue was suspended in ethanol, and a precipitated solid was collected by filtration and was dried under a reduced pressure, and the compound A-2 (10.7 g, yield: 79%) as a white powder was obtained.

MS (ESI); m/z 440.3 [M−H]−

(2) (+)-1,2-bis((2S,5S)-2,5-diethylphosphorano)benzene(1,5-cyclooctadiene)rhodium(I) tetrafluoroborate ((S,S)-Et-DUPHOS-Rh) (144 mg) was added to a mixture of the compound A-2 (9.65 g) and tetrahydrofuran (80 mL), and the mixture was stirred under a pressurized hydrogen atmosphere (800 kPa) at 35° C. for 3 hours. The reaction mixture was passed through silica gel column chromatography (solvent: hexane/(ethyl acetate)=50/50), and the solvent of the filtrate was distilled away under a reduced pressure. The residue was suspended in ethanol, and a precipitated solid was collected by filtration and was dried under a reduced pressure, and the compound A-3 (9.00 g, yield: 93%) as a white powder was obtained.

MS (ESI); m/z 442.2 [M−H]−

(3) The compound A-3 (7.52 g) was dissolved in a mixed solvent of tetrahydrofuran (40 mL), methanol (20 mL) and distilled water (15 mL), and a 4M aqueous lithium hydroxide solution (20 mL) was added under ice-cooling, and the mixture was stirred at 0° C. for 7 hours. 1M hydrochloric acid (60 mL) was added to the reaction mixture, and extraction with ethyl acetate (100 mL) was performed. An organic layer was washed with a saturated solution of sodium chloride, and was dried over magnesium sulfate, and insoluble matter was filtered, and the solvent was distilled away under a reduced pressure, and thereby, the compound A-4 (7.52 g, 88 wt %, yield: 100%) as a yellow viscous substance was obtained.

MS (ESI); m/z 386.2 [M−H]−

(4)-1 Cesium carbonate (3.97 g) and benzyl bromide (2.40 mL) were added at room temperature to a mixture of the compound A-4 (7.46 g, 88 wt %) and N,N-dimethylformamide (4 mL), and the mixture was stirred at the same temperature for 4 hours. A saturated solution of sodium chloride and water were added to the reaction mixture, and extraction with ethyl acetate was performed. An organic layer was washed with water and a saturated solution of sodium chloride, and the solvent was distilled away under a reduced pressure. The obtained residue was purified by silica gel column chromatography (solvent: hexane/(ethyl acetate)=80/20-67/33), and thereby, the compound A-5 (8.81 g, 90 wt %, 98%) as a white powder was obtained.

MS (ESI); m/z 476.2 [M−H]−

(4)-2 (Another synthesis method of the compound A-5) Iodine (153 mg) was added to a mixture of activated zinc (923 mg) and N,N-dimethylformamide (7 mL) at 5° C. under a nitrogen atmosphere. The temperature was raised to 20° C. and the mixture was stirred for 10 minutes. The reaction mixture was cooled again to 6° C., and the compound A-6 (1890 mg) was added in portions at 20° C. or lower, and the mixture was stirred at 20° C. for 30 minutes, and thereby, a solution of the compound A-7 was obtained.

Tris(dibenzylideneacetone)dipalladium(0)-chloroform adduct (31 mg), 2-dicyclohexylphosphino-2′,6′-dimethoxybiphenyldicyclohexyl(2′,6′-dimethoxy-[1,1′-biphenyl]-2-yl) phosphine (30 mg), and the compound A-8 (1309 mg) were sequentially added, and the mixture was stirred at room temperature for 16 hours. Hexane/(ethyl acetate) (1:1) was added to the reaction mixture, and insoluble matter was removed by celite filtration. The insoluble matter was washed with hexane/(ethyl acetate) (1:1) and water, and the filtrate was washed sequentially with a saturated aqueous ammonium chloride solution and a saturated solution of sodium chloride. An organic layer was dried over anhydrous magnesium sulfate, and insoluble matter was filtered, and then, the solvent was distilled away under a reduced pressure. The obtained residue was purified by silica gel column chromatography (solvent: hexane/(ethyl acetate)=80/20-67/33), and thereby, the compound A-5 (1.84 g, yield: 90%) as a yellow viscous substance was obtained.

MS (ESI); m/z 378.2 [M+H-Boc]+

(5) A 4M hydrogen chloride dioxane solution (6 mL) was added under ice-cooling to a mixture of the compound A-5 (1.63 g) and 1,4-dioxane (15 mL), and the mixture was stirred at room temperature for 1 hour. A 4M hydrogen chloride dioxane solution (6 mL) was added, and the mixture was stirred at room temperature for 16 hours. The reaction mixture was concentrated under a reduced pressure until the volume thereof was about 1/10. The residue was suspended in ethyl acetate, and a precipitated solid was collected by filtration and was dried under a reduced pressure, and thereby, the compound [a-1] (1288 mg, yield: 90%) as a white powder was obtained.

MS (ESI); m/z 378.4 [M+H]+

### Reference Example 41: Production of (2S)-2-amino-3-(3,4-diacetoxyphenyl) benzyl Propanoate; hydrochloride

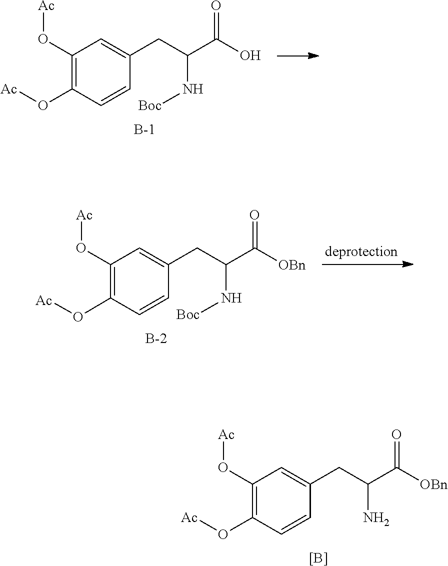

(1) Cesium carbonate (1.43 g) and benzyl bromide (0.58 mL) were added to a mixture of the compound B-1 (2.0 g) and N,N-dimethylformamide (19 mL), and the mixture was stirred at room temperature for 2 hours. A saturated solution of sodium chloride and water were added to the reaction mixture, and extraction with ethyl acetate was performed. An organic layer was washed with water and a saturated solution of sodium chloride and was dried over magnesium sulfate, and insoluble matter was filtered, and then, the solvent was distilled away under a reduced pressure. The obtained residue was purified by silica gel column chromatography, and thereby, the Compound B-2 (1.68 g, yield: 75%) as a colorless viscous substance was obtained.

MS (ESI); m/z 372.1 [M+H-Boc]+

(2) A 4M hydrogen chloride ethyl acetate solution (0.5 mL) was added under ice-cooling to a solution of the compound B-2 (515 mg) in ethyl acetate (5 mL), and the mixture was stirred at room temperature for 4 hours. A 4M hydrogen chloride ethyl acetate solution (3 mL) was added, and the mixture was stirred at room temperature for 2.5 hours. The solvent of the reaction mixture was distilled away under a reduced pressure. The residue was suspended in ethyl acetate, and a precipitated solid was collected by filtration and was dried under a reduced pressure, and thereby, the compound B (409 mg, yield: 98%) as a white powder was obtained.

MS (ESI); m/z 372.1 [M+H]+

### Reference Example 42: Production of (2S)-2-(benzyloxycarbonylamino)-3-(2,2-dimethyl-1,3-benzodioxol-5-yl) propanoic acid

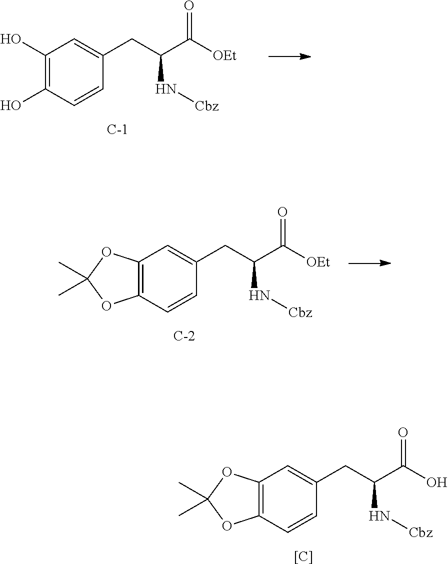

p-toluenesulfonic acid (175 mg) and 2,2-dimethoxypropane (12.5 mL) were added to a mixture of the compound C-1 (3.66 g) and toluene (102 mL), and the mixture was heated to reflux using a Dean-Stark apparatus for 14 hours. A saturated aqueous sodium hydrogen carbonate solution was added to the reaction mixture, and extraction with ethyl acetate was performed. An organic layer was dried over magnesium sulfate, and insoluble matter was filtered, and then, the solvent was distilled away under a reduced pressure, and a crude product of the compound C-2 as a yellow viscous substance was obtained.

The crude product of the compound C-2 was dissolved in a mixed solvent of methanol (25 mL), water (20 mL) and tetrahydrofuran (51 mL), and lithium hydroxide monohydrate (855 mg) was added under an ice bath, and the mixture was stirred at the same temperature for 40 minutes, and then, the mixture was stirred at room temperature for 1 hour. The reaction mixture was diluted with water and was washed with diethyl ether. Ethyl acetate was added to an aqueous layer, and a saturated aqueous citric acid solution was added until the pH of the mixture reached 6, and extraction with ethyl acetate was performed. An organic layer was dried over magnesium sulfate and insoluble matter was filtered, and then, the solvent was distilled away under a reduced pressure, and the residue was purified by silica gel column chromatography (solvent: ethyl acetate), and thereby, the compound C (2.40 g, 2-step yield: 63%) as a yellow viscous substance was obtained.

MS (ESI); m/z 370.2 [M−H]−

### Reference Example 43: Production of (2S)-2-(benzyloxycarbonylamino)-3-(2-ethoxy-2-methyl-1,3-benzodioxol-5-yl) propanoic acid

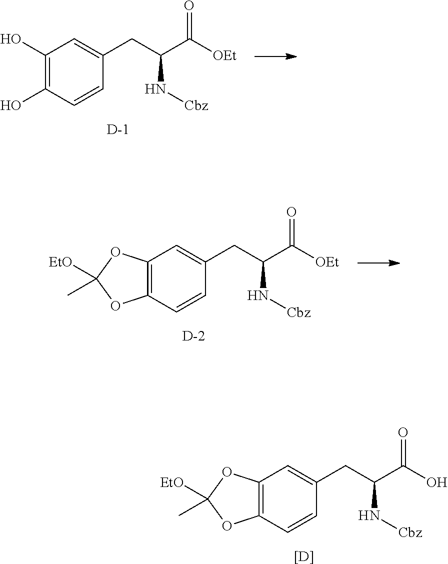

(1) The compound D-1 (1.35 g) was dissolved in 1-butyl-3-methylimidazolium hexafluorophosphate (7.51 mL), and triethyl orthoacetate (1.38 mL) was added, and the mixture was stirred at 80° C. for 3 hours. Water was added to the reaction mixture, and then, extraction with ethyl acetate was performed. An organic layer was dried over magnesium sulfate and insoluble matter was filtered, and then, the solvent was distilled away under a reduced pressure, and the residue was purified by silica gel column chromatography (solvent: hexane/(ethyl acetate)=85/15-70/30), and thereby, the compound D-2 (1.41 g, yield: 87%) as a colorless viscous substance was obtained.

MS (ESI); m/z 430.0 [M+H]+

(2) The compound D-2 (1.35 g) was dissolved in a mixed solvent of methanol (3.9 mL), water (6.3 mL) and tetrahydrofuran (6.3 mL), and lithium hydroxide monohydrate (264 mg) was added under an ice bath, and then, the mixture was stirred at room temperature for 10 hours. After adding ethyl acetate to the reaction mixture, a saturated aqueous ammonium chloride solution was added until the pH of the mixture reached 6, and extraction with ethyl acetate was performed. An organic layer was dried over magnesium sulfate and insoluble matter was filtered, and then, the solvent was distilled away under a reduced pressure, and the residue was purified by silica gel column chromatography (solvent: (ethyl acetate)/methanol=100/0-95/5), and thereby, the compound D (816 mg, yield: 65%) as a colorless solid was obtained.

MS (ESI); m/z 400.2 [M−H]−

### Experimental Example 1: Evaluation of In Vitro Conversion Efficiency Using Human Hepatocytes

Conversion efficiency from a prodrug to levodopa was evaluated with a metabolic study using human hepatocytes. A prodrug was incubated with human hepatocytes at 37° C. for 4 hours. A part of the reaction solution was sampled at each predetermined time and mixed with an organic solvent to stop the reaction. The reaction-stopped solution was centrifuged, and the obtained supernatant was analyzed using a liquid chromatography/tandem mass spectrometry method. The conversion efficiency to levodopa was evaluated as a levodopa production amount after 4 hours of incubation. Table 8 shows the levodopa production amounts of the compounds as some of the examples of the present invention.

TABLE 8

| Example | Levodopa production amount (nmol/L) |
|---|---|
| 1 | 862 |
| 2 | 1688 |
| 4 | 552 |
| 6 | 561 |
| 8 | 963 |
| 10 | 1083 |
| 17 | 574 |

As shown in the results of the above tests, it was confirmed that all the compounds produced levodopa. From these results, efficient levodopa production in vivo is expected, and it is considered to be particularly useful as a therapeutic medicament for Parkinson's disease.

### Experimental Example 2-1: Evaluation of Solution Stability

A phosphate buffer solution at pH 7.4 was added to each of the compounds to dissolve the each of the compounds, and when necessary, a NaOH solution was added to adjust the pH of the mixture to prepare a solution of about 1 mg/mL. After storing this solution at 25° C. for about 1 day, HPLC purity (area percentage, %) was measured. The HPLC purity of the solution immediately after the preparation, or the HPLC purity of the compound, was subtracted from the HPLC purity after storage at 25° C. for about 1 day to obtain a HPLC purity difference. Table 9 shows the solution stabilities of the compounds of some of the examples of the present invention. In Tables 9-1 and 9-2, “LDP-Ala” represents L-Tyrosine,N-L-alanyl-3-hydroxy-(9CI) and has the following structural formula:

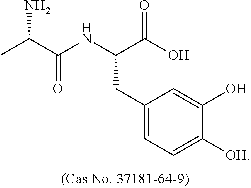 [SMILES: C[C@H](N)C(=O)N[C@@H](Cc1ccc(O)c(O)c1)C(=O)O]

TABLE 9-1

| Example | HPLC purity difference (%) 25° C., after about 1 day |
|---|---|
| 1 | −0.03 |
| 2 | −3.53 |
| 8 | 0.25 (after 1.9 days) |
| 10 | −0.67 |
| LDP-Ala | −3.80 |
| (Comparative | |
| Example) | |

### Experimental Example 2-2: Evaluation of Solution Stability

A phosphate buffer solution at pH 7.4 was added to each of the compounds to dissolve the each of the compounds, and when necessary, a NaOH solution was added to adjust the pH of the mixture to prepare a solution of about 1 mg/mL. After storing this solution at 60° C. for about 1 day, HPLC purity (area percentage, %) was measured. The HPLC purity of the solution immediately after the preparation, or the HPLC purity of the compound, was subtracted from the HPLC purity after storage at 60° C. for about 1 day to obtain a HPLC purity difference. Table 9-2 shows the solution stabilities of the compounds of some of the examples of the present invention.

TABLE 9-2

| Example | HPLC purity difference (%) 60° C., after about 1 day |
|---|---|
| 1 | −1.61 |
| LDP-Ala | −25.00 |
| (Comparative | |
| Example) | |

As shown in the results of the above tests, it was confirmed that the compounds of the present invention were stable in aqueous solutions near neutrality, and among them, the compounds of Example 1 and Example 8 were stable.

From the above results, the compounds of the present invention are expected to have high solution stability at pH 6-8, and are considered to be useful as a solution formulation.

### Experimental Example 3: Evaluation of Solubility

Water was added to each of the compounds to prepare a suspension, which was shaken at 25° C. for 24 hours, and then, filtration through a filter was performed to obtain a filtrate. A compound concentration in the filtrate was quantified using HPLC to obtain the solubility. Further, the pH of the filtrate was measured. Table 10-1 shows the solubilities of the compounds of some of the examples of the present invention.

TABLE 10-1

| Example | Solubility (mg/mL) | pH of filtrate |
|---|---|---|
| 1 | 15.6 | 2.6 |
| 2 | >31.1 | 2.6 |

As shown in the results of the above tests, it was confirmed that all the compounds showed good solubility at pH near the isoelectric point.

From the above results, the compounds are expected to have high solubility at pH 6-8, and are considered to be particularly useful as a solution formulation.

### (Preparation Method of Formulation)

### Preparation Method of LDP-Ala/CD Formulation

A NaOH solution was added to LDP-Ala and stirred under room temperature to dissolve LDP-Ala. Carbidopa and antioxidants (N-acetylcysteine and ascorbic acid) were added to the mixture and dissolved while adjusting pH by adding a NaOH solution as needed. Then, Polysorbate 80 and a NaOH solution were added to make the formulation composition in Table 10-2, and the volume was adjusted with ultrapure water as appropriate. The above solution was filtered through a 0.22 μm filter and filled into a vial while the headspace was replaced with nitrogen.

TABLE 10-2

| Components (%) | LDP-Ala/CD Formulation |
|---|---|
| LDP-Ala | 40 |
| Carbidopa | 1.3 |
| N-Acetylcysteine | 2.0 |
| Ascorbic acid | 2.0 |
| NaOH | q.s. |
| Polysorbate 80 | 0.3 |
| pH | 9.0 |

### Preparation Method of LDP-Lys/CD Formulation

A KOH solution and Meglumine were added to LDP-Lys and stirred under room temperature to dissolve LDP-Lys. Carbidopa and antioxidants (N-acetylcysteine and ascorbic acid) were added to the mixture and dissolved while adjusting pH by adding a KOH solution as needed. Then, Polysorbate 80 and a KOH solution were added to make the formulation composition in Table 10-3, and the volume was adjusted with ultrapure water as appropriate. The above solution was filtered through a 0.22 μm filter and filled into a vial while the headspace was replaced with nitrogen.

TABLE 10-3

| Components (%) | LDP-Lys/CD formulation |
|---|---|
| LDP-Lys | 33 |
| Carbidopa | 1.3 |
| N-Acetylcysteine | 2.0 |
| Ascorbic acid | 2.0 |
| KOH | q.s. |
| Meglumine | q.s. |
| Polysorbate 80 | 0.3 |
| pH | 9.0 |

### (Results of Stability Evaluation of LDP-Ala and LDP-Lys Formulations)

The formulations prepared as shown in Table 10-2 and Table 10-3 were kept refrigerated (2-8° C.) for 2 weeks. Afterwards, the appearance of each of the formulations was visually checked and evaluated for the presence of precipitates. HPLC was also used to determine the quantification and purity of LDP-Ala and LDP-Lys.

In both formulations shown in Table 10-2 and Table 10-3, no precipitation was observed after 2 weeks of refrigeration, and no significant change was observed in the quantitative values, indicating that the formulations were considered stable up to 2 weeks of refrigeration. The stability evaluation results of the formulations shown in Table 10-2 and Table 10-3 are shown in Table 10-4 and Table 10-5, respectively.

TABLE 10-4

| Appearance | Initial | 5° C. 2 W |
|---|---|---|
| LDP-Ala/CD formulation | No precipitation | No precipitation |
| LDP-Lys/CD formulation | No precipitation | No precipitation |

TABLE 10-5

| Quantitative value (purity %) | Initial | 5° C. 2 W |
|---|---|---|
| LDP-Ala/CD formulation | 416.3(91.8%) | 418.3(91.8%) |
| LDP-Lys/CD formulation | 334.2(90.8%) | 327.3(91.4%) |

### Part II: LD-Tyr Formulations

### Experimental Example 4—Long-Term Stability of 30% LD-Tyrosine/0.75% Carbidopa at 2-8° C.

A formulation comprising 300 mg/mL (30%) LD-tyrosine (LD-Tyr) and 7.5 mg/mL (0.75%) carbidopa was prepared with 5.5% L-Arginine and 11.5% Tris. The formulation also included 0.5% ascorbic acid (Asc), 0.5% N-acetyl cysteine (NAC) and 0.3% Tween 80. The formulation was stored at 2-8° C. for the time indicated, and % recovery and % LD-Tyr-DKP formation (an impurity) were tested. Results are shown in Table 11. Percent recovery remained high over time. The DKP impurity accumulated over time.

TABLE 11
Stability of LD-Tyr and Carbidopa

| | LD-Tyrosine | | | Carbidopa | |
|---|---|---|---|---|---|
| | | % | % DKP | | % |
| Timepoint | mg/mL | Recovery | % area | mg/mL | Recovery |
| t = 0 | 297.62 | | 0.86 | 7.37 | |
| 1 week | 294.13 | 98.83 | 0.99 | 7.36 | 99.86 |
| 2 weeks | 304.25 | 102.23 | 1.19 | 7.30 | 99.05 |
| 1 month | 295.74 | 99.37 | 1.88 | 7.32 | 99.32 |
| 2 months | 302.47 | 101.63 | 2.60 | 7.32 | 99.32 |
| 3 months | 296.57 | 98.74 | 4.04 | 7.35 | 99.73 |

### Experimental Example 5—Solubility in Cosolvents

The solubility of the LD-tyrosine (LD-Tyr) was tested in various cosolvents, as indicated in Table 12. The formulation was stored at 2-8° C. or 25° C. for the time indicated, and stability (as measured by the presence of precipitation) and % DKP formation (an impurity) were tested. As shown in Table 12, the formulation with propylene glycol was stable for at least up to 1 month at 2-8° C. and at least up to 2 weeks at 25° C., and showed less DKP accumulation than the formulation comprising 5.5% L-Arginine and 11.5% Tris shown in Example 1. The formulation with PEG 300 was unstable at 2-8° C. and showed more DKP accumulation than the other formulations tested. Formulations comprising DMA at 5%, 10% and 15% were also prepared. Significant levels of DKP were detected at T=0 for all concentrations.

TABLE 12
Cosolvents

| | | | | | | Physical |
|---|---|---|---|---|---|---|
| | Max. | | % DKP | % DKP | Stability | Stability |
| | Conc. | % DKP | 2 wks at | 2 wks at | 1 month at | 2 weeks at |
| Cosolvent | (mg/ml) | (t = 0) | 2-8° C. | 25° C. | 2-8° C. | 25° C. |
| Ethanol | >10 | NA | NA | NA | NA | NA |
| Propylene | 81.98 | 0.38 | 0.64 | 2.51 | No evidence of | No evidence of |
| glycol | | | | | precipitation | precipitation |
| PEG 300 | 49.11 | 1.58 | 2.82 | 11.71 | Precipitated | No evidence of |
| | | | | | after 2 weeks | precipitation |
| Dimethyl- | 84.37 | 41.39 | NA | NA | NA | NA |
| acetamide | | | | | | |
| (DMA) | | | | | | |

### Experimental Example 6—Effect of Various Additives on Stability of a 30% LD-Tyrosine-L-Arginine Based Formulation

Formulations were prepared with 300 mg/mL LD-Tyr, 5 mg/mL (0.5%) NAC, 5 mg/mL (0.5%) ascorbic acid, 18.1% L-Arg, and the additive shown in Table 13. PVP K17 stands for polyvinylpyrrolidone low molecular weight (PVP K17). All formulations were physically stable for at least two weeks at 2-8° C. and 25° C. As shown in Table 13, none of the additives tested showed ability to inhibit or prevent DKP formation.

TABLE 13
Additives

| | | | | | | | Physical | Physical |
|---|---|---|---|---|---|---|---|---|
| | | | | | | | Stability | Stability |
| | | % DKP | % DKP (2-8° C.) | | % DKP (25° C.) | | 2 weeks/ | 2 weeks/ |
| Ingredient | pH | (t = 0) | 1 week | 2 weeks | 1 week | 2 weeks | 2-8° C. | 25° C. |
| 18.1% L- | 8.44 | 0.16 | 0.33 | 0.53 | 1.22 | 2.40 | No evidence of | No evidence of |
| Arginine | | | | | | | precipitation | precipitation |
| 18.1% L- | 8.24 | 0.18 | 0.39 | 0.58 | 1.28 | 2.66 | No evidence of | No evidence of |
| Arg + 0.5% | | | | | | | precipitation | precipitation |
| MgCl₂ | | | | | | | | |
| 18.1% L- | 8.36 | 0.17 | 0.36 | 0.54 | 1.19 | 2.47 | No evidence of | No evidence of |
| Arg + 4% | | | | | | | precipitation | precipitation |
| PVP K17 | | | | | | | | |
| 18.1% L- | 8.35 | 0.18 | 0.38 | 0.55 | 1.22 | 2.52 | No evidence of | No evidence of |
| Arg + 0.5% | | | | | | | precipitation | precipitation |
| MgCl₂ + 4% | | | | | | | | |
| PVP K17 | | | | | | | | |
| 18.1% L- | 8.28 | 0.17 | 0.35 | 0.57 | 1.24 | 2.59 | No evidence of | No evidence of |
| Arg + 5% | | | | | | | precipitation | precipitation |
| Mannitol | | | | | | | | |

### Experimental Example 7—Solubility and Physical Stability in Bases (Organic and Inorganic)

In this Example, LD-Tyrosine was added in portions to an 0.8M solution of counter ion and antioxidants, while stirring. The L-arginine formulation was heated while stirring, while the other formulations were not. pH was not adjusted. Solubility was confirmed following filtration by HPLC. Physical stability was measured after 2 weeks and 1 month of storage at 2-8° C. or 25° C. As shown in Table 14, solubilizing LD-Tyrosine in ethanolamine, diethylamine, and ammonium hydroxide resulted in physical stability (no evidence of precipitation) at 2-8° C. and at 25° C. up to at least one month. Solubility and stability were dependent upon the particular base used and not only upon pH.

TABLE 14
Solubility and Physical Stability as a Function of Bases

| | | | Physical Stability | | Physical Stability | |
|---|---|---|---|---|---|---|
| 0.8M bases | Solubility | | 2-8° C. | | 25° C. | |
| in WFI | (mg/ml) | pH | 2 weeks | 1 month | 2 weeks | 1 month |
| L-Arginine | 290 | 7.95 | No evidence of | Precipitated | No evidence of | Precipitated |
| | | | precipitation | | precipitation | |
| Meglumine | 280 | 8.02 | Precipitated | Precipitated | No evidence of | Precipitated |
| | | | | | precipitation | |
| Ethylenediamine | 330 | 8.0 | Precipitated | Precipitated | Precipitated | Precipitated |
| Ethanolamine | 135 | 9.34 | No evidence of | No evidence of | No evidence of | No evidence of |
| | | | precipitation | precipitation | precipitation | precipitation |
| Diethylamine | 185 | 9.26 | No evidence of | No evidence of | No evidence of | No evidence of |
| | | | precipitation | precipitation | precipitation | precipitation |
| Sodium | 130 | 9.96 | Precipitated | Precipitated | No evidence of | No evidence of |
| Hydroxide | | | | | precipitation | precipitation |
| Ammonium | 135 | 9.7 | No evidence of | No evidence of | No evidence of | No evidence of |
| Hydroxide | | | precipitation | precipitation | precipitation | precipitation |

In addition to stability, impurity (DKP) formation was measured in each formulation. As shown in Table 15, DKP formation was lowest in formulations using sodium hydroxide at 25° C. DKP formation was slightly higher in diethylamine, ethanolamine, and ammonium hydroxide, respectively.

TABLE 15
DKP Formation as a Function of Bases

| | | | % | % DKP | % DKP | % DKP | % DKP |
|---|---|---|---|---|---|---|---|
| 0.8M bases | Solubility | | DKP | 2 weeks/ | 1 month/ | 2 weeks/ | 1 month/ |
| in WFI | (mg/ml) | pH | t = 0 | 2-8° C. | 2-8° C. | 25° C. | 25° C. |
| L-Arginine | 290 | 7.95 | 0.34 | 0.66 | 1.53 | 3.04 | NA |
| Meglumine | 280 | 8.02 | 0.36 | NA | NA | 3.47 | NA |
| Ethylenediamine | 330 | 8.0 | 0.37 | NA | NA | NA | NA |
| Ethanolamine | 135 | 9.34 | 0.27 | 0.46 | 0.65 | 1.67 | 3.09 |
| Diethylamine | 185 | 9.26 | 0.23 | 0.41 | 0.60 | 1.50 | 2.83 |
| Sodium | 130 | 9.96 | 0.20 | NA | NA | 0.81 | 1.30 |
| Hydroxide | | | | | | | |
| Ammonium | 135 | 9.7 | 0.30 | 0.49 | 0.69 | 1.79 | 3.17 |
| Hydroxide | | | | | | | |

Formulations were prepared with 300 mg/mL LD-Tyr, 5 mg/mL (0.5%) NAC and 5 mg/mL (0.5%) ascorbic acid and either 17.2% Meglumine or 18.1% L-Arg. All formulations were physically stable for at least two weeks at 2-8° C. and 25° C. As shown in Table 16, a lower level of DKP was observed when L-Arg was used relative to Meglumine.

TABLE 16
Meglumine

| | | | | | | | Physical | Physical |
|---|---|---|---|---|---|---|---|---|
| | | % DKP | % DKP | % DKP | % DKP | % DKP | Stab 2 | Stab 2 |
| | | level | 1 wk at | 2 wks at | 1 wk at | 2 wks at | weeks at | weeks at |
| Ingredient | pH | (t = 0) | 2-8° C. | 2-8° C. | 25° C. | 25° C. | 2-8° C. | 25° C. |
| 17.2% | 8.29 | 0.18 | 0.43 | 0.69 | 1.62 | 3.12 | No evidence of | No evidence of |
| Meglumine | | | | | | | precipitation | precipitation |
| 18.1% L- | 8.44 | 0.16 | 0.33 | 0.53 | 1.22 | 2.4 | No evidence of | No evidence of |
| Arginine | | | | | | | precipitation | precipitation |

### Experimental Example 8—Effect of Various Amines and Inorganic Bases on the Stability of 30% LD-Tyrosine

In this Example, 300 mg/mL (30%) LD-Tyrosine formulations were prepared with the ingredients indicated in Table 17, as well as the antioxidants ascorbic acid (0.5%) and N-acetyl cysteine (NAC) (0.5%). No heating was used to prepare these formulations.

TABLE 17
Effect of ethylenediamine, ethanolamine, diethanolamine, and diethylamine on DKP

| | | | | | | | Physical | Physical |
|---|---|---|---|---|---|---|---|---|
| | | | % DKP | % DKP | % DKP | % DKP | Stability | Stability |
| | | % DKP | 1 week at | 2 weeks at | 1 week at | 2 week at | 2 week at | 2 weeks at |
| Ingredient | pH | (t = 0) | 2-8° C. | 2-8° C. | 25° C. | 25° C. | 2-8° C. | 25° C. |
| 4.25% | 8.35 | 0.19 | 0.58 | 0.88 | 2.7 | 4.97 | No evidence of | No evidence of |
| Ethylenediamine | | | | | | | precipitation | precipitation |
| 7.2% L-Arg + | 8.51 | 0.17 | 0.44 | 0.65 | 2.04 | 3.77 | No evidence of | No evidence of |
| 3% | | | | | | | precipitation | precipitation |
| Ethylenediamine | | | | | | | | |
| 7.2% L-Arg + | 8.82 | 0.17 | 0.35 | 0.50 | 1.38 | 2.67 | No evidence of | No evidence of |
| 4.5% | | | | | | | precipitation | precipitation |
| Ethanolamine | | | | | | | | |
| 7.2% L-Arg + | 8.65 | 0.17 | 0.35 | 0.49 | 1.4 | 2.72 | No evidence of | No evidence of |
| 7.7% | | | | | | | precipitation | precipitation |
| Diethanolamine | | | | | | | | |
| 7.2% L-Arg + | 8.91 | 0.16 | 0.3 | 0.4 | 1.06 | 2.01 | No evidence of | No evidence of |
| 5.4% | | | | | | | precipitation | precipitation |
| Diethylamine | | | | | | | | |

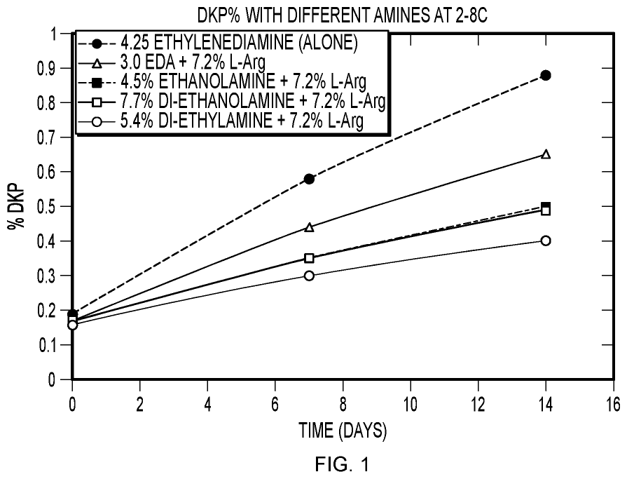

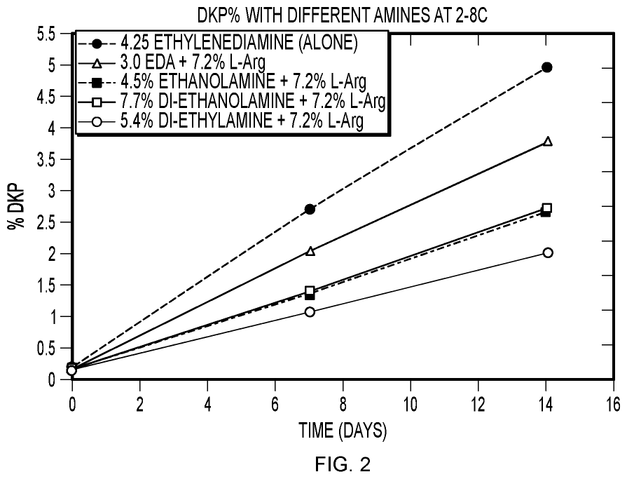

As shown in Table 17 and in FIGS. 1 and 2, the formulation comprising diethylamine and L-Arg resulted in the lowest DKP level formation, followed by diethanolamine and L-Arg and ethanolamine and L-Arg. Of the amines tested, the highest amount of DKP was seen in formulations comprising ethylenediamine and L-Arg and ethylenediamine alone (no L-Arg). Relative DKP level formation was similar at 2-8° C. and at 25° C.

Formulations were prepared with 300 mg/mL LD-Tyr, 5 mg/mL (0.5%) NAC and 5 mg/mL (0.5%) ascorbic acid and the combinations of bases indicated in Table 18. All formulations were physically stable for at least 70 days at 2-8° C.

TABLE 18
Tris (Tromethamine)

| | | | % DKP | % DKP | |
|---|---|---|---|---|---|
| | | % DKP | (45 days/ | 70 days/ | Physical Stability |
| Combination | pH | (t = 0) | 2-8° C.) | 2-8° C.) | (70 days/2-8° C.) |
| 11.5% Tris + | 8.27 | 0.22 | 2.44 | 3.34 | No evidence of |
| 5.5% L-Arg | | | | | precipitation |
| 7.2% Tris + | 8.33 | 0.22 | 2.06 | 2.84 | No evidence of |
| 10.2% L-Arg | | | | | precipitation |
| 3.6% Tris + | 8.37 | 0.22 | 1.78 | 2.43 | No evidence of |
| 15% L-Arg | | | | | precipitation |
| 0% Tris + | 8.44 | 0.24 | 1.68 | 2.25 | No evidence of |
| 18.1% L-Arg | | | | | precipitation |

Formulations were prepared with 300 mg/mL LD-Tyr, 5 mg/mL (0.5%) NAC, 5 mg/mL (0.5%) ascorbic acid, 7.2% L-Arg, and either sodium hydroxide or ammonium hydroxide as indicated in Table 19. As shown in Table 19, formulations comprising sodium hydroxide demonstrated physical instability.

TABLE 19
Sodium and Ammonium Hydroxide

| | | | | | Physical |
|---|---|---|---|---|---|
| | | | % DKP | Physical | Stability |
| | | % DKP | 1 week/ | Stability | 2 weeks/ |
| Ingredient | pH | (t = 0) | 2-8° C. | 25° C. | 2-8° C. |
| 7.2% L-Arg + | 8.99 | NA | NA | Precipitated | NA |
| 2.95% NaOH | | | | immediately | |
| 7.2% L-Arg + | 8.46 | NA | NA | Precipitated | NA |
| 2.0% NaOH | | | | the day after | |
| | | | | preparation | |
| 7.2% L-Arg + | 9.42 | NA | NA | Precipitated | NA |
| 2.6% NH₄OH | | | | the day after | |
| | | | | preparation | |
| 7.2% L-Arg + | 8.32 | 0.12 | 0.28 | No evidence of | No evidence |
| 1.5% NH₄OH | | | | precipitation | of |
| | | | | after one week | precipitation |

### Experimental Example 9—Effect of Ethanolamine on the Stability of 30% LD-Tyrosine

In this Example, 300 mg/mL (30%) LD-Tyrosine formulations were prepared with L-Arginine and ethanolamine alone and in combination at different concentrations as shown in Table 20, as well as the antioxidants ascorbic acid (0.5%) and N-acetyl cysteine (NAC) (0.5%). Only the formulation containing 18.1% L-Arg was heated during preparation.

TABLE 20
Ethanolamine

| Bases | | Molar ratio | | |
|---|---|---|---|---|
| (solubilizer | | (Total bases - | Solids | Density |
| ingredient) | pH | Antiox):LD-Tyr | (%) | (g/ml) |
| 18.1% L-Arginine | 8.44 | 1.17:1.0 | 49.0 | 1.136 |
| 7.2% L-Arginine + | 8.56 | 1.21:1.0 | 38.2 | 1.107 |
| 4.0% Ethanolamine | | | | |
| 5% L-Arginine + | 8.62 | 1.16:1.0 | 36.0 | 1.104 |
| 4.5% Ethanolamine | | | | |
| 5% L-Arginine + | 8.35 | 1.06:1.0 | 36.0 | 1.104 |
| 4% Ethanolamine | | | | |
| 5.75% Ethanolamine | 8.42 | 1.06:1.0 | 31.0 | 1.095 |
| (No L-Arginine) | | | | |

The formulations were then tested for DKP accumulation and physical stability at 2-8° C. and 25° C. over the course of 2 weeks. As shown in Table 21, formulations containing L-Arg alone had the lowest level of DKP accumulation, and the amount of DKP accumulation generally increased with increasing ratio of ethanolamine to L-Arg. No evidence of precipitation was seen in any formulations tested. Only the formulation containing 18.1% L-Arg was heated during preparation.

TABLE 21
DKP Formation in Ethanolamine

| Bases | | | % DKP | % DKP | Physical stab. | Physical stab. |
|---|---|---|---|---|---|---|
| (solubilizer | | % DKP | 2 weeks/ | 2 weeks/ | 2 weeks/ | 2 weeks/ |
| ingredient) | pH | T = 0 | 2-8° C. | 25° C. | 2-8° C. | 25° C. |
| 18.1% Arginine | 8.44 | 0.16 | 0.53 | 2.4 | No evidence of | No evidence of |
| | | | | | precipitation | precipitation |
| 7.2% Arg + | 8.56 | 0.21 | 0.58 | 3.04 | No evidence of | No evidence of |
| 4.0% Ethanolamine | | | | | precipitation | precipitation |
| 5% L-Arg + | 8.62 | 0.22 | 0.62 | 3.24 | No evidence of | No evidence of |
| 4.5% Ethanolamine | | | | | precipitation | precipitation |
| 5% L-Arg + | 8.35 | 0.18 | 0.60 | 3.37 | No evidence of | No evidence of |
| 4% Ethanolamine | | | | | precipitation | precipitation |
| 5.75% Ethanolamine | 8.42 | 0.16 | 0.68 | 3.62 | No evidence of | No evidence of |
| | | | | | precipitation | precipitation |

Table 22 presents additional 300 mg/mL (30%) LD-Tyrosine formulations prepared with L-Arginine and ethanolamine or diethylamine, as well as the antioxidants ascorbic acid (0.5%) and N-acetyl cysteine (NAC) (0.5%).

TABLE 22

| | | LD-Tyrosine assay (mg/ml) 2-8° C. | | | | | | | | |
|---|---|---|---|---|---|---|---|---|---|---|
| | | | | | | 1 mo % | DKP (% area) 2-8° C. | | | |
| Formulation | pH | t = 0 | 1 wk | 2 wks | 1 mo | recovery | t = 0 | 1 wk | 2 wks | 1 mo |
| 11.4% Arg + 4.0% | 8.93 | 295.57 | 302.70 | 287.59 | 295.57 | 100.0 | 0.2 | 0.30 | 0.40 | 0.78 |
| Ethanolamine | | | | | | | | | | |
| 5% Arg + 6.25% | 8.94 | 294.5 | 302.4 | 288.9 | 292.8 | 99.4 | 0.21 | 0.37 | 0.49 | 0.93 |
| Ethanolamine | | | | | | | | | | |
| 5% Arg + 7.0% | 9.11 | 292.2 | 300.9 | 288.2 | 292.5 | 100.1 | 0.23 | 0.35 | 0.46 | 0.87 |
| Ethanolamine | | | | | | | | | | |
| 7.2% Arg + 5.4% | 9.02 | ND | 300.93 | 290.75 | 295.76 | 98.3 | 10.2 | 0.26 | 0.36 | 0.7 |
| Diethylamine | | | | | | | | | | |

### Experimental Example 10—Blood/Plasma Ratio

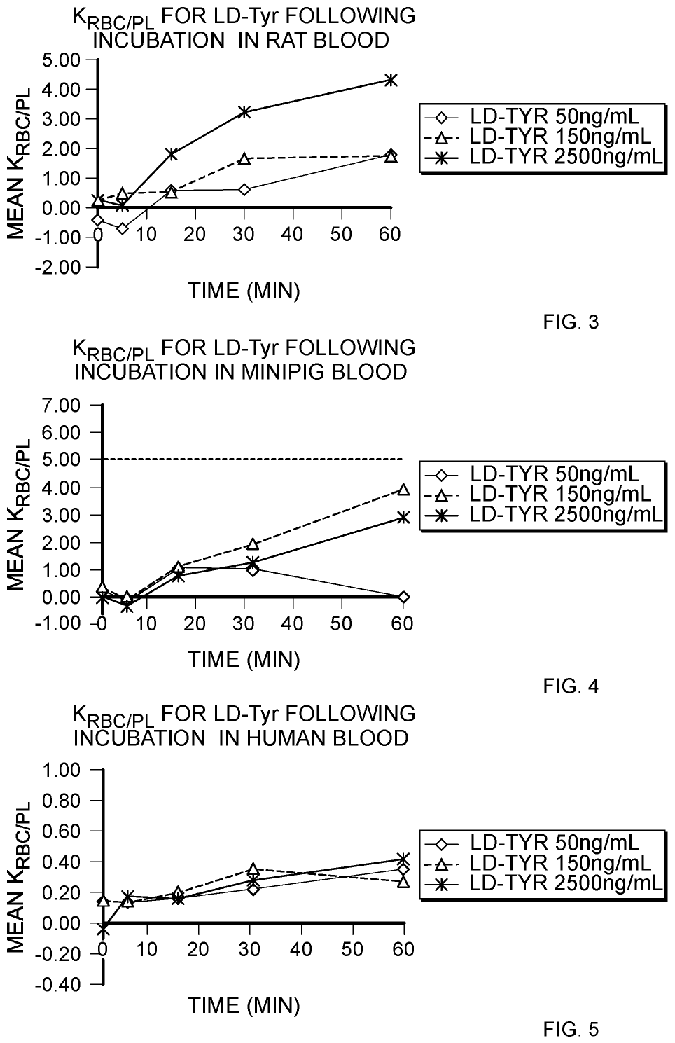

Blood/plasma ratio (R_{B}) of LD-Tyr at a concentration of 50, 150, and 2500 ng/mL, was investigated ex vivo in rat, minipig, and human blood, to obtain an understanding of partitioning into red blood cells (RBC). In rat and minipig blood, the values of the partition coefficient between RBC and plasma (K_{RBC/PL}) were below five, indicating limited RBC binding. In rat blood, K_{RBC/PL} increased with the time of incubation (see FIG. 3). In minipig blood, K_{RBC/PL} increased with the time of incubation for the two highest concentrations (150 and 2500 ng/mL), while for the lowest concentration (50 ng/mL), after reaching 1 at 15 and 30 minutes, the K_{RBC/PL} declined to 0.0 (see FIG. 4). In human blood, all K_{RBC/PL} measured were below one, indicating no RBC binding (see FIG. 5).

### Experimental Example 11—Safety Studies in Rats and Minipigs

In vivo non-clinical safety studies were conducted in both the rat and minipig. Additionally, an assessment of the local infusion site reaction of LD-Tyr was performed as part of a PK study in domestic pigs. A summary of the in vivo non-clinical safety results obtained in various non-clinical studies of LD-Tyr is presented in Table 23.

TABLE 23
Summary of In Vivo Non-Clinical Safety Results

| Study | Study | Species/ | | | |
|---|---|---|---|---|---|
| No. | Type | Strain | Dosing regimen | LD-Tyr Dose | Noteworthy findings |
| MTD Exp | Maximal | Rat/SD | Bolus SC | 90, 150, 300, | Mortality (1/4 males) |
| 001 | Tolerated | | | 625 mg/kg | and moderate irritation |
| | Dose | | | | at the injections site (all |
| | (MTD) | | | | animals) at the highest |
| | | | | | dose. |
| | | | | | MTD = 300 mg/kg |
| 0NMR6 | Local | Minipig/ | 24-hour SC | 120 mg/mL | LD-Tyr and its |
| | safety | Göttingen | infusion | at 4.5 mL | respective vehicle were |
| | | | (240 μL/h × 16 h + | (540 mg/day) | well tolerated with no |
| | | | 80 μL/h × 8 h) | | evidence of any adverse |
| | | | | | local effect and no |
| | | | | | cutaneous ulcers; |
| | | | | | minimal to mild |
| | | | | | subchronic/chronic |
| | | | | | inflammation in the |
| | | | | | subcutis. No systemic |
| | | | | | findings. |
| 0NMR10 | Local | Minipig/ | 24-hour SC | 130 mg/mL | LD-Tyr and its |
| | safety | Göttingen | infusion | at 6 mL (780 | respective vehicle were |
| | | | (315 μL/h × 18 h + | mg/day) | well tolerated with no |
| | | | 50 μL/h × 6 h) | | evidence of any adverse |
| | | | | | local effect and no |
| | | | | | cutaneous ulcers; |
| | | | | | minimal to moderate |
| | | | | | chronic inflammation in |
| | | | | | the subcutis. No |
| | | | | | systemic findings. |
| ND 2-20- | PK | Pig/ | 18-hour SC | 200 mg/mL | LD-Tyr was well |
| 102 | | domestic | infusion | at 9 mL | tolerated with no |
| | | | (500 μL/h) | (1800 | evidence of any adverse |
| | | | | mg/day) | local effect and no |
| | | | | | cutaneous ulcers; |
| | | | | | minimal to mild |
| | | | | | subacute inflammation |
| | | | | | in the subcutis. |
| E0515 | Local | Minipig/ | 24-hour SC | 440, 370, | LD-Tyr and its |
| | safety | Göttingen | infusion | 300, and 250 | respective vehicle were |
| | | | (320, 400, and | mg/mL at 6, | well tolerated with no |
| | | | 485 μL/h × 18 h + | 7.5, 6 and 6 | evidence of any adverse |
| | | | 50 μL/h × 6 h) | mL, | local effect and no |
| | | | | respectively | cutaneous ulcers; mild |
| | | | | (2640, 2775, | to moderate |
| | | | | 2700, and | subchronic/chronic |
| | | | | 2250 | inflammation in the |
| | | | | mg/day, | subcutis. No systemic |
| | | | | respectively) | findings. |

### Experimental Example 12—Effect of Various Additives on Stability of a 20% LD-Tyrosine-L-Arginine Based Formulation

Formulations were prepared with 200 mg/mL LD-Tyr, 5 mg/mL (0.5%) NAC, 5 mg/mL (0.5%) ascorbic acid, 15.5% L-Arg, and the additives as shown in Table 24. As shown in Table 24, the tested additives were able to limit the DKP formation at 2-8° C.; however, the amount of DKP at room temperature remained significant. It is further noted that the formulations comprising MgCl₂ demonstrated relatively high viscosity. The solubility of the LD-Tyr was DMA>Ethanol>PEG 300, and the viscosity of the formulations was Ethanol<DMA<PEG 300.

TABLE 24

| Ingredients (mg/mL) | F1 | F2 | F3 | F4 | F5 |
|---|---|---|---|---|---|
| LD-Tyr | 200 | 200 | 200 | 200 | 200 |
| Arg | 155 | 155 | 155 | 155 | 155 |
| NAC | 5 | 5 | 5 | 5 | 5 |
| Ascorbic acid | 5 | 5 | 5 | 5 | 5 |
| hydroxypropyl beta | 200 | | | | |
| cyclodextrin (HPBCD) | | | | | |
| sulfobutyl-ether beta | | 200 | | | |
| cyclodextrin (SBECD) | | | | | |
| Lysine | | | 200 | | |
| ethanol | | | | 155.5 | |
| PEG 300 | | | | | 226 |
| MgCl₂ | | | | | 100 |
| pH | 8.77 | 8.98 | 9.76 | 8.84 | 7.88 |
| DKP increase 1 M 2-8° | 0.15 | 0.15 | 0.16 | 0.21 | 0.12 |
| C. (% area) | | | | | |
| DKP increase 1 M 25° | 2.93 | 3 | 2.33 | 2.7 | 1.5 |
| C. (% area) | | | | | |

### Experimental Example 13—Effect of Ethanol and/or PEG 300 on Formulation Characteristics

The stability and the saturation of the formulations in Table 25 was assessed. F1 proved to be stable for 1 week at 5° C., even when increasing the amount of LD-Tyr to 400 mg/ml. F2-F4 on the other hand, did not remain stable when the amount of LD-Tyr was increased to 400 mg/ml. In order to maintain physical stability, L-Arginine was added to formulations F2-F4, and the pH was adjusted to the original level. After doing so, F2-F4 were able to remain stable for 1 week at 5° C., even when increasing the amount of LD-Tyr to 400 mg/ml.

TABLE 25

| Ingredient (mg/mL) | Role | F1 | F2 | F3 | F4 |
|---|---|---|---|---|---|
| Levodopa-Tyr | API | 300 | 300 | 300 | 300 |
| Carbidopa | API | 13 | 13 | 13 | 13 |
| L-Arginine | pH | 240 | 200 | 200 | 200 |
| | adjustment/Counter | | | | |
| | ion | | | | |
| Ethanol | Co-solvent | 155.5 | 116.6 | 0 | 116.6 |
| PEG 300 | Co-solvent | 0 | 0 | 100 | 50 |
| N-Acetyl Cysteine | Antioxidant | 10 | 10 | 10 | 10 |
| WFI | Solvent | QS | QS | QS | QS |
| pH | | 9.0 | 8.7 | 8.7 | 8.7 |

Visual inspection after one month also confirmed the physical stability of the formulations in Table 25 (the formulations remained clear, and no precipitations were evident). The stability was tested by HPLC as well, and the results showed ˜100% recovery after 5 days. In addition, one month stability HPLC tests, on formulations that were held at either 2-8° C. or 25° C. for one month showed that the stability was maintained under both conditions. The main difference between the formulations is in the amount of DKP formed over time, wherein at 2-8° C. the formation of DKP is inhibited, while at 25° C. it is not. In this respect it is noted that the amount of DKP formed in F1 is lower than that formed in any of F2-F4.

### Experimental Example 14—Pharmacokinetics of LD-Tyr in Domestic Pigs

A series of PK studies was completed in domestic pigs using several LD-Tyr formulations, as detailed in Table 26, and dosing regimens, as detailed in Table 27. The PK profile of LD-Tyr as well as the LD resulting therefrom was determined following 18-hour continuous SC infusion to female domestic pigs. Each formulation was administered at one infusion site per animal using a Crono-ND infusion pump (Canè SpA Medical Technology, Rivoli, Italy) connected to an infusion set (Accu-chek®, FlexLink, Roche).

Blood samples for the determination of LD-Tyr and LD plasma levels were collected from the pigs at the following timepoints:

0 (pre-dose), 30 min, 1 h, 2 h, 6 h, 8 h, 9 h, 10 h, 12 h, 14 h, 18 h, 18.5 h, 19 h, 21 h, 23 h and 26 h following the start of the infusion.

TABLE 26

| | | LD-Tyr | | |
|---|---|---|---|---|
| | LD-Tyr | 13% | | |
| | 13% | (ND-2- | | |
| | (ND-2-20- | 20-102 | LD-Tyr | LD-Tyr |
| | 101 Study) | Study) | 20% | 30% |
| Test Item (w/v %) | Formulation Composition (w/v %) | | | |
| Levodopa | 13 | 13 | 20 | 30 |
| Tyrosine | | | | |
| Carbidopa | 0.75 | 0.75 | 0.75 | 0.75 |
| monohydrate (dry | | | | |
| basis) | | | | |
| L-Arginine | 0.0 | 0.0 | 0.0 | 18.10 |
| NMP | 24.7 | 24.7 | 0 | |
| Polysorbate 80 | 0.3 | 0.3 | 0.3 | 0.30 |
| Ascorbic acid | 0.2 | 0.2 | 0.5 | 0.50 |
| N-Acetylcysteine | 0.0 | 0.2 | 0.5 | 0.50 |
| (NAC) | | | | |
| NaOH | 2.8 | 1.01 | 1.01 | 0.0 |
| pH | 7.59 | 7.61 | 8.6 | 8.36 |

A summary of the PK parameters, as well as the dosing regimen for each study, is detailed in Table 27.

TABLE 27
Dosing Regimen and Summary of the PK Profile of LD-Tyr and LD
Following Continuous Subcutaneous Infusion in the Domestic Pig

| | LD-Tyr | | | | PK parameters | | | |
|---|---|---|---|---|---|---|---|---|
| | Concentration | LD-Tyr | | | (CV %) | | | |
| | in Dosing | Daily | Infusion | | | | | AUC₀₋ₜ |
| | Solution | dose | volume | Dosing | | Cₘₐₓ | tₘₐₓ | (ng × |
| Study No. | (mg/mL) | (mg/day) | (mL) | regimen | Analyte | (ng/mL) | (h) | h/mL) |
| ND 2-20- | 130 | 780 | 6 | 18-hour SC | LD- | 99 | 10.4 | 1331 |
| 101 | | | | infusion at | Tyr | (93.5) | (55.1) | (89.2) |
| (n = 5) | | | | 335 μL/h | LD | 1915 | 17.0 | 31150 |
| | | | | | | (9.9) | (16.6) | (8.4) |
| ND 2-20- | 130 | 1170 | 9 | 18-hour SC | LD- | 143 | 12.0 | 1799 |
| 102 | | | | infusion at | Tyr | (NA) | (NA) | (NA) |
| (n = 2) | | | | 500 μ/h | LD | 1952 | 15.3 | 33167 |
| | | | | | | (NA) | (NA) | (NA) |
| ND 2-20- | 200 | 1800 | 9 | 18-hour SC | LD- | 312 | 11.3 | 4532 |
| 102 | | | | infusion at | Tyr | (NA) | (NA) | (NA) |
| (n = 3) | | | | 500 μL/h | LD | 3142 | 18.3 | 54295 |
| | | | | | | (NA) | (NA) | (NA) |
| ND 2-20- | 300 | 1800 | 6 | 18-hour SC | LD- | 300 | 14.0 | 3765 |
| 103 | | | | infusion at | Tyr | (38.6) | (17.5) | (39.7) |
| (n = 5) | | | | 335 μL/h | LD | 4765 | 17.6 | 74877 |
| | | | | | | (9.9) | (11.6) | (5.8) |
| ND 2-20- | 300 | 2700 | 9 | 18-hour SC | LD- | 403 | 11.4 | 5387 |
| 103 | | | | infusion at | Tyr | (53.4) | (11.8) | (59.4) |
| (n = 5) | | | | 500 μL/h | LD | 7843 | 17.4 | 121456 |
| | | | | | | (10.2) | (11.0) | (15.9) |

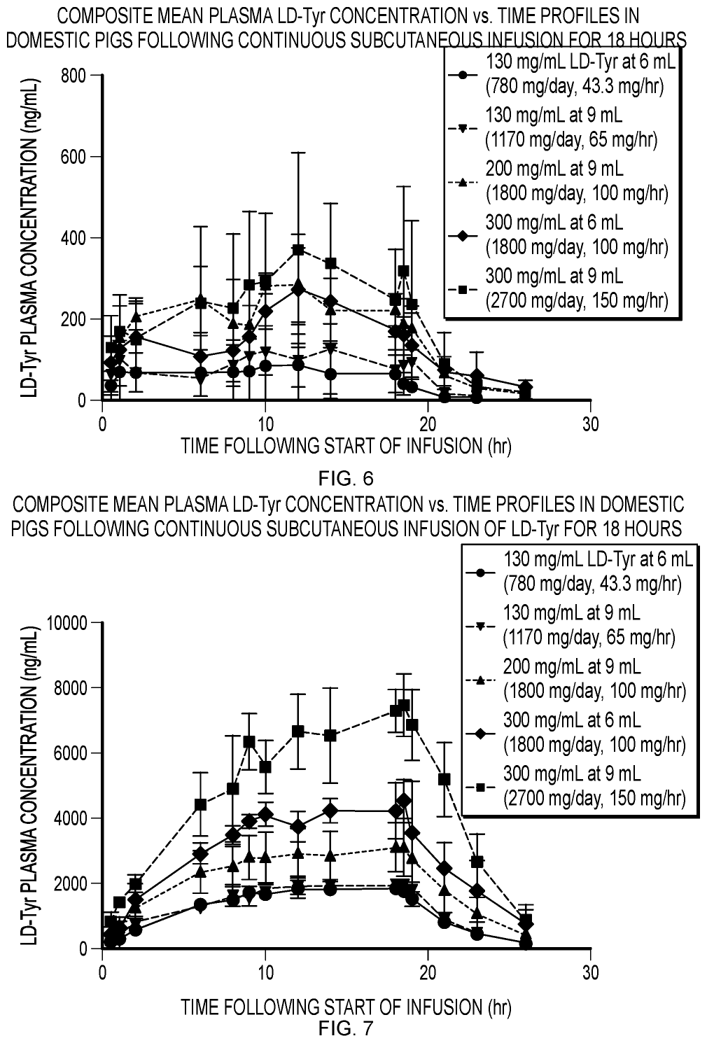

The composite mean concentrations of LD-Tyr and resulting LD, resulting from the studies detailed above, are depicted in FIGS. 6 and 7, respectively.

### Experimental Example 15—Pharmacokinetic and Local Site Reaction Studies on High Concentration LD-Tyr Formulations Comprising TRIS in Domestic Pigs

### Experimental Example 15A—1% CD

A series of PK and local-tox studies was completed in domestic pigs using several high concentration LD-Tyr formulations comprising 1% CD and varying amounts of TRIS, as detailed in Table 28, and dosing regimens, as detailed in Table 29. The PK profile of LD-Tyr as well as the LD resulting therefrom was determined following 18-hour continuous SC infusion to female domestic pigs. Each formulation was administered at one infusion site per animal using a Crono-ND infusion pump (Canè SpA Medical Technology, Rivoli, Italy) connected to an infusion set (Accu-chek®, FlexLink, Roche).

Blood samples for the determination of LD-Tyr and LD plasma levels were collected from the pigs at the following timepoints:

0 (pre-dose), 30 min, 1 h, 2 h, 6 h, 8 h, 9 h, 10 h, 12 h, 14 h, 18 h, 18.5 h, 19 h, 21 h, 23 h and 26 h following the start of the infusion.

TABLE 28

| Test Item (w/v %) | LD-Tyr 30% | LD-Tyr 37% | LD-Tyr 44% |
|---|---|---|---|
| LD-Tyrosine | 30 | 37 | 44 |
| Carbidopa | 1.00 | 1.00 | 1.00 |
| Polysorbate 80 | 0.30 | 0.30 | 0.30 |
| Ascorbic acid | 0.50 | 0.50 | 0.50 |
| N-Acetylcysteine (NAC) | 0.50 | 0.50 | 0.50 |
| Tromethamine (TRIS) | 11.50 | 13.00 | 15.00 |
| L-Arginine | 5.50 | 6.50 | 8.00 |
| WFI | 67.5 | 61.0 | 52.80 |
| Final pH | 8.17 | 8.20 | 8.14 |

TABLE 29

| | | LD-Tyr/ CD | Daily dose | | In- | In- |
|---|---|---|---|---|---|---|
| | | Nominal | (LD- | Infusion | fusion | fusion |
| | Test | concentration | Tyr/CD | rate | volume | duration |
| N= | item | (% w/v) | mg/day) | (μL/hr) | (mL) | (hour) |
| 5 | LD-Tyr | 30/1 | 2700/90 | 500 | 9 | 18 |
| 5 | LD-Tyr | 37/1 | 2775/75 | 415 | 7.5 | 18 |
| 5 | LD-Tyr | 44/1 | 2640/60 | 335 | 6 | 18 |

### PK Results

A summary of the PK parameters obtained in provided in Table 30.

TABLE 30

| | LD-Tyr/CD | | | | | | |
|---|---|---|---|---|---|---|---|
| | Nominal | Number | LD-Tyr/CD | | PK parameters | | |
| Test | Concentration | of | Daily dose | | Cₘₐₓ | tₘₐₓ | AUCₜ |
| Item | (% w/v) | animals* | (mg) | Analyte | (ng/mL) | (h) | (ng*h/mL) |
| LD-Tyr | 30/1 | n = 3 | 2700/90 | LD-Tyr | 144.4 | 11.33 | 1151.3 |
| | | | | LD | 3877.7 | 15.33 | 66682 |
| | 37/1 | n = 3 | 2755/75 | LD-Tyr | 203.9 | 23 | 666.7 |
| | | | | LD | 5418.4 | 17.33 | 83736 |
| | 44/1 | n = 1 | 2640/60 | LD-Tyr | 236.51 | 18 | 3499.9 |
| | | | | LD | 5129.5 | 18 | 73609 |

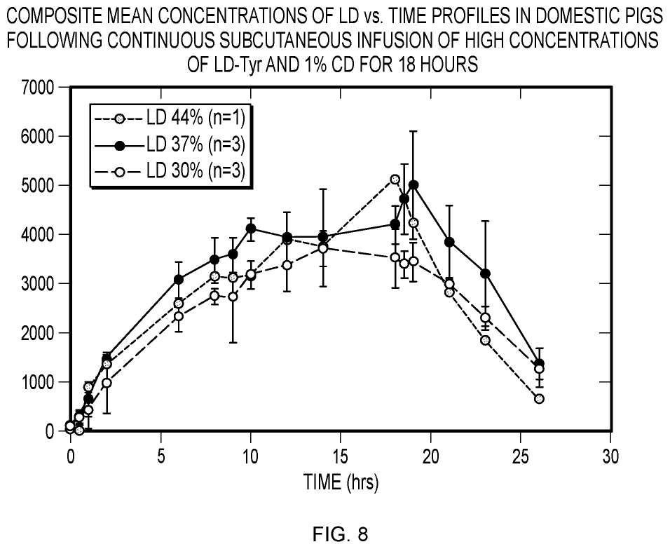

The composite mean concentrations of LD, resulting from the studies detailed above, are depicted in FIG. 8.

Based on the results and under the conditions of this study, it can be concluded that no significant difference in systemic exposure to LD was observed following infusion of LD-Tyr 30% at 9 mL, LD-Tyr 37% at 7.5 mL, or LD-Tyr 44% at 6 mL.

### Local Site Reaction

Infusion sites were examined for any local reaction immediately after pump removal. Any dermal reaction (e.g. erythema, edema/swelling) was recorded and scored according to a 5-point Draize scale for scoring skin irritation as provided in Table 31.

TABLE 31

| | Erythema and | Edema |
|---|---|---|
| Score | eschar formation | formation |
| 0 | No erythema | No edema |
| 1 | Very slight erythema | Very slight edema |
| | (barely perceptible) | (barely perceptible) |
| 2 | Well-defined erythema | Well defined edema |
| | | (edges of area |
| | | welldefined by |
| | | definite raising) |
| 3 | Moderate erythema | Moderate edema (raised |
| | | approximately 1 mm) |
| 4 | Severe erythema (beet redness) | Severe edema (raised |
| | to eschar formation | more than 1 mm |
| | preventing grading | andextending |
| | of erythema | beyond exposure area) |

The results showed that no significant local skin reactions were induced by any of the test formulations. Very slight to well-defined erythema and edema (grade 1-2) was observed immediately following pump removal, as shown in Table 32.

TABLE 32

| | Erythema and eschar | | | | | Edema formation | | | | |
|---|---|---|---|---|---|---|---|---|---|---|
| | formation score | | | | | score | | | | |
| Test Item | 0 | 1 | 2 | 3 | 4 | 0 | 1 | 2 | 3 | 4 |
| LD-Tyr 30% | 2 | 0 | 3 | 0 | 0 | 5 | 0 | 0 | 0 | 0 |
| LD-Tyr 37% | 2 | 3 | 0 | 0 | 0 | 2 | 1 | 2 | 0 | 0 |
| LD- Tyr 44% | 4 | 1 | 0 | 0 | 0 | 4 | 1 | 0 | 0 | 0 |

### Experimental Example 15B—0.5% CD

A series of PK and local-tox studies was completed in domestic pigs using several high concentration LD-Tyr formulations comprising 0.5% CD and varying amounts of TRIS, as detailed in Table 33, and dosing regimens, as detailed in Table 34. The PK profile of LD-Tyr as well as the LD resulting therefrom was determined following 18-hour continuous SC infusion to female domestic pigs. Each formulation was administered at one infusion site per animal using a Crono-ND infusion pump (Canè SpA Medical Technology, Rivoli, Italy) connected to an infusion set (Accu-chek®, FlexLink, Roche).

Blood samples for the determination of LD-Tyr and LD plasma levels were collected from the pigs at the following timepoints:

0 (pre-dose), 30 min, 1 h, 2 h, 6 h, 8 h, 9 h, 10 h, 12 h, 14 h, 18 h, 18.5 h, 19 h, 21 h, 23 h and 26 h following the start of the infusion.

TABLE 33

| Test Item (w/v %) | LD-Tyr 30% | LD-Tyr 37% | LD-Tyr 44% |
|---|---|---|---|
| LD-Tyrosine | 30 | 37 | 44 |
| Carbidopa | 0.50 | 0.50 | 0.50 |
| Polysorbate 80 | 0.30 | 0.30 | 0.30 |
| Ascorbic acid | 0.50 | 0.50 | 0.50 |
| N-Acetylcysteine (NAC) | 0.50 | 0.50 | 0.50 |
| L-Arginine | 5.50 | 6.50 | 8.00 |
| Tromethamine (TRIS) | 11.50 | 13.00 | 15.00 |
| WFI | 69.25 | 61.50 | 53.00 |
| Final pH | 8.25 | 8.13 | 8.17 |

TABLE 34

| | | LD-Tyr/CD | | | | |
|---|---|---|---|---|---|---|
| | | Nominal | Daily dose | Infusion | Infusion | Infusion |
| | Test | concentration | (LD-Tyr/ | rate | volume | duration |
| N= | item | (% w/v) | CDmg/day) | (μL/hr) | (mL) | (hour) |
| 5 | LD-Tyr | 37/0.5 | 2775/37.5 | 415 | 7.5 | 18 |
| 5 | LD-Tyr | 44/0.5 | 2640/30 | 335 | 6 | 18 |
| 5 | LD-Tyr | 30/0.5 | 2700/45 | 500 | 9 | 18 |

### PK Results

A summary of the PK parameters obtained in provided in Table 35.

TABLE 35

| | LD-Tyr/CD | | | | | | |
|---|---|---|---|---|---|---|---|
| | Nominal | LD-Tyr/CD | | Number | PK parameters | | |
| Test | Concentration | Daily dose | | of | Cmax | tmax | AUCt |
| Item | (% w/v) | (mg) | Analyte | animals* | (ng/mL) | (h) | (ng*h/mL) |
| LD-Tyr | 30/0.5 | 2775/37.5 | LD-Tyr | n = 2 | 444.8 | 10.0 | 4799.5 |
| | | | LD | | 3648.0 | 16.25 | 55206.5 |
| | 37/0.5 | 2640/30 | LD- Tyr | n = 3 | 490.6 | 15.0 | 5602.1 |
| | | | LD | | 3404.6 | 18.33 | 51791.0 |
| | 44/0.5 | 2700/45 | LD- Tyr | n = 1 | 49.6 | 2 | 534.3 |
| | | | LD | | 2020.7 | 14 | 33736.0 |

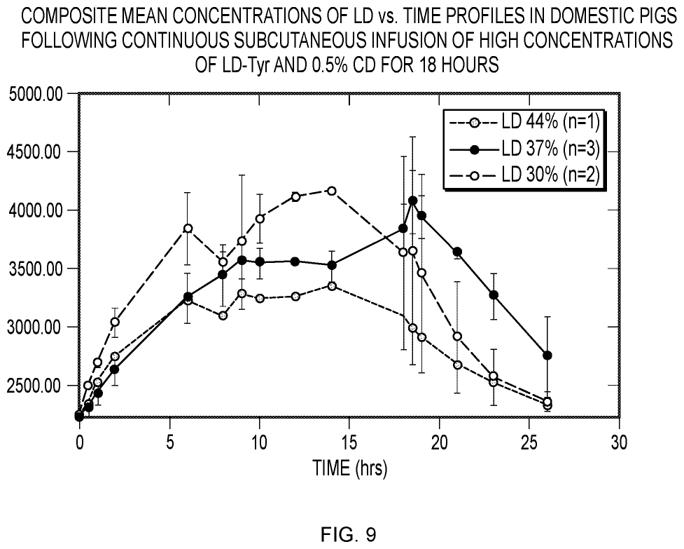

The composite mean concentrations of LD, resulting from the studies detailed above, are depicted in FIG. 9.

Based on the results and under the conditions of this study, it can be concluded that no significant difference in systemic exposure to LD was observed following infusion of LD-Tyr 30% at 9 mL, LD-Tyr 37% at 7.5 mL, or LD-Tyr 44% at 6 mL.

### Local Site Reaction

Infusion sites were examined for any local reaction immediately after pump removal. Any dermal reaction (e.g. erythema, edema/swelling) was recorded and scored according to a 5-point Draize scale for scoring skin irritation as provided in Table 31.

No significant local skin reactions were observed in most treated sites. The reactions that were observed immediately following pump removal, were very slight to moderate (grade 1-3) erythema and edema, as shown in Table 36.

TABLE 36

| | Erythema and | | | | | Edema formation | | | | |
|---|---|---|---|---|---|---|---|---|---|---|
| | escharformation score | | | | | score | | | | |
| Test Item | 0 | 1 | 2 | 3 | 4 | 0 | 1 | 2 | 3 | 4 |
| LD-Tyr 30% | 0 | 3 | 1 | 1 | 0 | 0 | 2 | 2 | 1 | 0 |
| LD-Tyr 37% | 2 | 1 | 1 | 1 | 0 | 0 | 3 | 1 | 1 | 0 |
| LD-Tyr 44% | 1 | 1 | 1 | 2 | 0 | 4 | 1 | 0 | 0 | 0 |

### Experimental Example 16—Pharmacokinetic and Local Site Reaction Studies in Domestic Pigs Administering Formulations with Varying Concentrations of LD-Tyr and CD

A series of PK and local site reaction studies was completed in domestic pigs using several concentrations LD-Tyr formulations comprising varying amounts of CD, as detailed in Table 37, and dosing regimens, as detailed in Table 38. The PK profile of LD-Tyr, as well as the LD resulting therefrom, was determined following 18-hour continuous SC infusion to female domestic pigs. Each formulation was administered at one infusion site per animal using a Crono-ND infusion pump (Canè SpA Medical Technology, Rivoli, Italy) connected to an infusion set (Accu-chek®, FlexLink, Roche).

Blood samples for the determination of LD-Tyr and LD plasma levels were collected from the pigs at the following timepoints:

0 (pre-dose), 30 min, 1 h, 2 h, 6 h, 8 h, 9 h, 10 h, 12 h, 14 h, 18 h, 18.5 h, 19 h, 21 h, 23 h and 26 h following the start of the infusion.

TABLE 37

| Test Item (w/v %) | LD-Tyr 30% | LD-Tyr 30% | LD-Tyr 44% |
|---|---|---|---|
| LD-Tyrosine base | 30 | 30 | 44 |
| Carbidopa | 0.50 | 1.50 | 1.0 |
| Polysorbate 80 | 0.30 | 0.30 | 0.30 |
| Ascorbic acid | 0.50 | 0.50 | 0.50 |
| N-Acetylcysteine (NAC) | 0.50 | 0.50 | 0.50 |
| L-Arginine | 18.10 | 18.10 | 25.70 |
| WFI | 65.33 | 65.14 | 53.51 |
| Final pH | 8.4 | 8.42 | 8.35 |

TABLE 38

| | | LD-Tyr/CD | | | | |
|---|---|---|---|---|---|---|
| | | Nominal | Daily dose | Infusion | Infusion | Infusion |
| | Test | concentration | (LD-Tyr/CD | rate | volume | duration |
| n= | item | (% w/v) | mg/day) | (μL/hr) | (mL) | (hour) |
| 5 | LD-Tyr | 30/0.5 | 2700/45 | 500 | 9 | 18 |
| 5 | LD-Tyr | 30/1.5 | 2700/135 | 500 | 9 | 18 |
| 5 | LD-Tyr | 44/1 | 2640/60 | 335 | 6 | 18 |

### PK Results

A summary of the PK parameters obtained in provided in Table 39.

TABLE 39

| | Nominal | | LD-Tyr/ | | | | |
|---|---|---|---|---|---|---|---|
| | Concentration | Number | CD Daily | | PK parameters | | |
| Test | LD-Tyr/CD | of | dose | | Cₘₐₓ | tₘₐₓ | AUCₜ |
| Item | (% w/v) | animals* | (mg) | Analyte | (ng/mL) | (h) | (ng*h/mL) |
| LD-Tyr | 30/0.5 | n = 5 | 2700/45 | LD-Tyr | 453.2 | 8.8 | 5454.0 |
| | | | | LD | 8027.0 | 12.8 | 116400.0 |
| | 30/1.5 | n = 5 | 2700/135 | LD-Tyr | 985.8 | 12.4 | 13517.0 |
| | | | | LD | 9287.8 | 18.9 | 142890.0 |
| | 44/1 | n = 4 | 2640/60 | LD-Tyr | 737.7 | 11.6 | 8131.2 |
| | | | | LD | 8406.4 | 15.1 | 127150.0 |

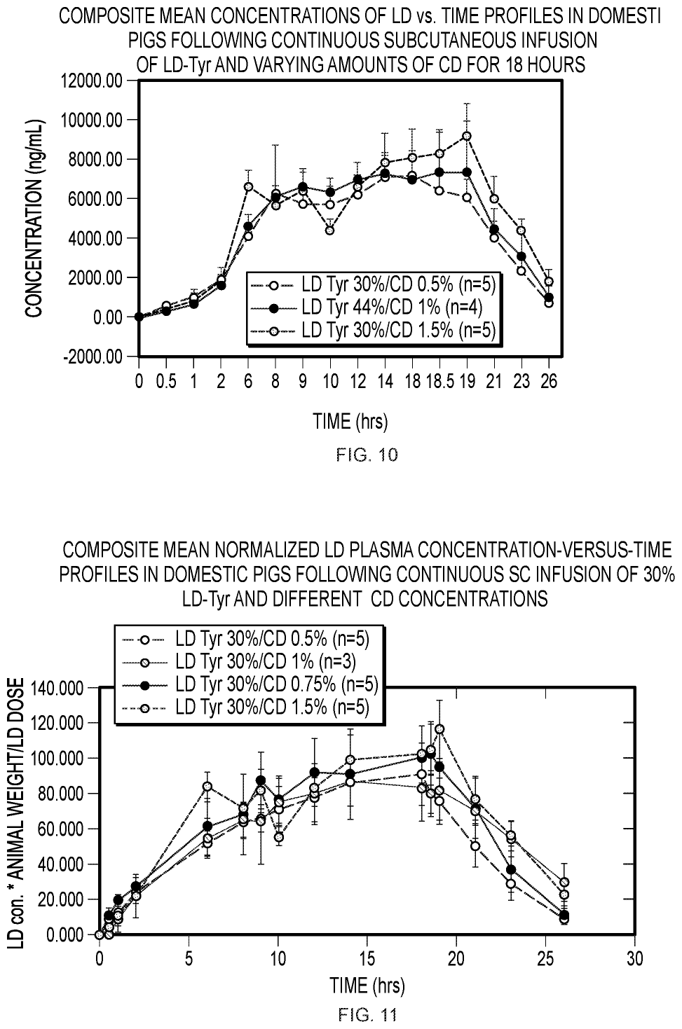

The composite mean concentrations of LD, resulting from the studies detailed above, are depicted in FIG. 10.

### Local Site Reaction

Infusion sites were examined for any local reaction immediately after pump removal. Any dermal reaction (e.g. erythema, edema/swelling) was recorded and scored according to a 5-point Draize scale for scoring skin irritation as provided in Table 31.

No significant local skin reactions were observed in all treated sites. The reactions that were observed immediately following pump removal, were very slight to well-defined erythema (grade 1-2), as shown in Table 40.

TABLE 40

| | Erythema and eschar | | | | | Edema formation | | | | |
|---|---|---|---|---|---|---|---|---|---|---|
| | formation score | | | | | score | | | | |
| Test Item | 0 | 1 | 2 | 3 | 4 | 0 | 1 | 2 | 3 | 4 |
| LD-Tyr 30%/CD 0.5% | 3 | 2 | 0 | 0 | 0 | 5 | 0 | 0 | 0 | 0 |
| LD-Tyr 30%/CD 1.5% | 3 | 2 | 0 | 0 | 0 | 5 | 0 | 0 | 0 | 0 |
| LD-Tyr 44%/CD 1% | 3 | 0 | 2 | 0 | 0 | 5 | 0 | 0 | 0 | 0 |

### Experimental Example 17—CD Titration

The effect of the dose of CD on LD systemic exposure was assessed by comparing the plasma concentrations of LD following continuous SC infusion of combinations of LD-Tyr 30% and different concentrations of CD (0.5-1.5%). The data were obtained from various preclinical studies (see Table 41) and were normalized according to animals' body weight. The composite mean normalized plasma concentrations of LD of are shown FIG. 11.

TABLE 41
LD-Tyr/CD Dose used for Assessment of CD Titration

| | | LD-Tyr/CD | |
|---|---|---|---|
| | Number of | Concentration | LD-Tyr/CD |
| Study No. | animals | (% w/v) | Daily dose (mg) |
| ND 2-20-106 | 5 | 30/0.5 | 2700/45 |
| ND 2-20-103 | 5 | 30/0.75 | 2700/67.5 |
| ND 2-20-104 | 3 | 30/1 | 2700/90 |
| ND 2-20-106 | 5 | 30/1.5 | 2700/135 |

It is noted that the results show that the LD plasma concentrations following infusion of LD-Tyr 30% with various CD concentrations were similar to one another, independent of the CD dose.

### Experimental Example 18—PK and Local Site Reaction Studies Following Bolus and Subcutaneous Administration of a 30% LD-Tyr Formulation

A series of PK and local-tox studies was completed in domestic pigs using several concentrations LD-Tyr formulations comprising varying amounts of CD, as detailed in Table 42, and dosing regimens, as detailed in Table 43. The PK profile of LD-Tyr, as well as the LD resulting therefrom, was determined following 18-hour continuous SC infusion to female domestic pigs. Each formulation was administered at one infusion site per animal using a Crono-ND infusion pump (Canè SpA Medical Technology, Rivoli, Italy) connected to an infusion set (Accu-chek®, FlexLink, Roche).

Blood samples for the determination of LD-Tyr and LD plasma levels were collected from the pigs at the following timepoints:

0 (pre-dose), 10 min (end of bolus), 15 min, 30 min, 45 min, 60 min, 75 min, 90 min, 120 min and 240 min following the beginning of the bolus administration.

TABLE 42

| Test Item (w/v %) | LD-Tyr 30% |
|---|---|
| LD-Tyrosine base | 30 |
| Carbidopa | 1.00 |
| Polysorbate 80 | 0.30 |
| Ascorbic acid | 0.50 |
| N-Acetylcysteine (NAC) | 0.50 |
| L-Arginine | 18.10 |
| WFI | 66.92 |
| Final pH | 8.42 |

TABLE 43

| | | LD-Tyr/CD | Bolus | | | Infusion | | | | Total |
|---|---|---|---|---|---|---|---|---|---|---|
| | | Nominal | | | LD-Tyr/ | | | | LD-Tyr/ | LD-Tyr/CD |
| | Test | Concentration | Volume | Duration | CD dose | Volume | Rate | Duration | CD dose | Daily Dose |
| n= | Item | (% w/v) | (mL) | (min) | (mg) | (mL) | (μL/hr) | (hr) | (mg) | (mg) |
| 5 | ND1601 | 30/1 | 0.7 | 10 | 210/7 | 1 | 485 | ~2 | 300/10 | 510/17 |
| 5 | ND1601 | 30/1 | 1.2 | 10 | 360/12 | 1 | 485 | ~2 | 300/10 | 660/22 |
| 5 | ND1601 | 30/1 | 1.7 | 10 | 510/17 | 1 | 485 | ~2 | 300/10 | 810/27 |

### PK Results

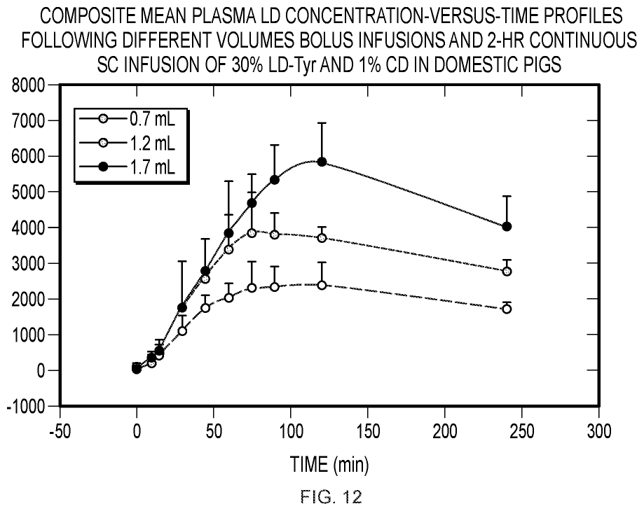

The composite mean PK parameters for LD-Tyr and resulting LD in domestic pigs are shown in Table 44. The composite mean plasma concentrations of LD-Tyr and resulting LD are depicted in FIG. 12.

TABLE 44
Composite PK Parameters following Bolus Infusion and 2-hr
continuous SC administration of LD-Tyr in Domestic Pigs

| | Nominal | | | | | | | |
|---|---|---|---|---|---|---|---|---|
| | Concentration | Bolus | LD-Tyr/CD | LD-Tyr/CD | | PK parameters | | |
| Test | LD/CD | Volume | bolus dose | Daily dose | | Cmax | tmax | AUCt |
| Item | (% w/v) | (mL) | (mg) | (mg) | Analyte | (ng/mL) | (h) | (ng*h/mL) |
| LD- | 30/1 | 0.7 | 210/7 | 510/17 | LD-Tyr | 572.9 | 1.3 | 1176.5 |
| Tyr | | | | | LD | 2574.6 | 2.2 | 7362.9 |
| | 30/1 | 1.2 | 360/12 | 660/22 | LD- Tyr | 933.3 | 0.9 | 1633.9 |
| | | | | | LD | 4264.6 | 1.4 | 11744.0 |
| | 30/1 | 1.7 | 510/17 | 810/27 | LD- Tyr | 1347.0 | 1.2 | 2432.2 |
| | | | | | LD | 5814.7 | 2 | 16638.0 |

Based on the results and under the conditions of this study, it can be concluded that increase of bolus injection volume from 0.7 mL to 1.2 mL and then to 1.7 mL, followed by a 2-hr continuous infusion at the same LD-Tyr/CD concentration (30%/1%), resulted in a dose-proportional increase in systemic exposure to LD.

The results also suggest that a bolus injection of LD-Tyr prior to continuous SC infusion may reduce LD tmax compared to continuous SC infusion of LD-Tyr without a bolus injection.

### Local Infusion Site Reactions

Infusion sites were examined for any local reaction immediately after pump removal. Any dermal reaction (e.g. erythema, edema/swelling) was recorded and scored according to a 5-point Draize scale for scoring skin irritation as provided in Table 31.

No significant local skin reactions were observed in all treated sites. The reactions that were observed immediately following pump removal, were very slight to well-defined edema (grade 1-2), as shown in Table 45.

TABLE 45

| | Erythema and eschar | | | | | Edema formation | | | | |
|---|---|---|---|---|---|---|---|---|---|---|
| | formation score | | | | | score | | | | |
| Test Item | 0 | 1 | 2 | 3 | 4 | 0 | 1 | 2 | 3 | 4 |
| LD-Tyr 30%/CD 1%, | 5 | 0 | 0 | 0 | 0 | 5 | 0 | 0 | 0 | 0 |
| 0.7 mL bolus | | | | | | | | | | |
| LD-Tyr 30%/CD 1%, | 5 | 0 | 0 | 0 | 0 | 0 | 4 | 1 | 0 | 0 |
| 1.2 mL bolus | | | | | | | | | | |
| LD-Tyr 30%/CD 1%, | 5 | 0 | 0 | 0 | 0 | 0 | 2 | 3 | 0 | 0 |
| 1.7 mL bolus | | | | | | | | | | |

### Experimental Example 19—Local Tolerability and Pathology Studies of Various Formulations Following 24 h SC Administration in Domestic Pigs

The local tolerability and pathology of injection sites following 24 h of SC administration in domestic pigs of the formulations and vehicles detailed in Table 46 according to the dosing regimen as detailed in Table 47 was tested.

TABLE 46

| Ingredient | Test Item | | | | | | | | | |
|---|---|---|---|---|---|---|---|---|---|---|
| (w/v %) | F1 | F2 | F3 | F4 | F5 | V1 | V2 | V3 | V4 | V5 |
| LD-Tyrosine | 30 | 30 | 30 | 30 | 30 | 0.00 | 0.00 | 0.00 | 0.00 | 0.00 |
| Carbidopa | 1.3 | 1.3 | 1.3 | 1.3 | 1.3 | 0.00 | 0.00 | 0.00 | 0.00 | 0.00 |
| monohydrate | | | | | | | | | | |
| L-Arginine | 22.60 | 12.40 | 5.20 | 8.00 | 7.20 | 22.60 | 12.40 | 5.20 | 8.00 | 7.20 |
| Ethanolamine | 0.00 | 4.00 | 6.50 | 0.00 | 0.00 | 0.00 | 4.00 | 6.50 | 0.00 | 0.00 |
| Diethanolamine | 0.00 | 0.00 | 0.00 | 9.00 | 0.00 | 0.00 | 0.00 | 0.00 | 9.00 | 0.00 |
| Diethylamine | 0.00 | 0.00 | 0.00 | 0.00 | 5.60 | 0.00 | 0.00 | 0.00 | 0.00 | 5.60 |
| Ascorbic acid | 0.50 | 0.50 | 0.50 | 0.50 | 0.50 | 0.50 | 0.50 | 0.50 | 0.50 | 0.50 |
| NAC | 0.50 | 0.50 | 0.50 | 0.50 | 0.50 | 0.50 | 0.50 | 0.50 | 0.50 | 0.50 |
| HCl 37% | 0.00 | | | | | HCl added in an amount to reach desired pH, | | | | |
| | | | | | | to be compared to corresponding formulation | | | | |
| WFI | 60.00 | 62.00 | 68.00 | 65.00 | 67.00 | 60.00 | 62.00 | 68.00 | 65.00 | 67.00 |
| Final pH | 8.66 | 8.88 | 8.92 | 8.72 | 8.75 | 8.81 | 8.77 | 8.77 | 8.85 | 8.71 |

TABLE 47

| | | LD-Tyr/CD | Infusion | Infusion | Infusion |
|---|---|---|---|---|---|
| Treatment | Formulation | Concentration | Volume | Rate | Duration |
| Code | Description | (mg/mL) | (mL) | (μL/h) | (h) |
| F1 | L-Arg | 300/13 | 9 | 485 × 18 h + | 24 |
| F2 | EthanolamineLD | | | 50 × 6 h | |
| F3 | EthanolamineHD | | | | |
| F4 | Diethylamine | | | | |
| F5 | Diethanolamine | | | | |
| V1 | L-Arg | 0 | | | |
| | Vehicle | | | | |
| V2 | EthanolamineLD | | | | |
| | Vehicle | | | | |
| V3 | EthanolamineHD | | | | |
| | Vehicle | | | | |
| V4 | Diethylamine | | | | |
| | Vehicle | | | | |
| V5 | Diethanolamine | | | | |
| | Vehicle | | | | |
| S1 | Saline | | | | |
| NG | Sham - Neria | 0 | N/A | N/A | N/A |
| | guard needle | | | | |
| | insertion | | | | |

### Results

Based on the histopathological results, it was concluded that ethanolamine, diethanolamine and diethylamine-based formulations (F2-F5 and V2-V5) were not tolerated following a 24-hr SC infusion at a volume of 9 mL. In contrast, the F1/V1 formulations, which are not amine-based, were tolerated.

### Experimental Example 20—Local Tolerability and Pathology Studies of Additional Formulations Following 24 h SC Administration in Domestic Pigs

The local tolerability and pathology of injection sites following 24 h of SC administration in domestic pigs of the formulations and vehicles detailed in Table 48 according to the dosing regimen as detailed in Table 49 was tested.

TABLE 48

| Ingredient | Test Item | | | | | | |
|---|---|---|---|---|---|---|---|
| (mg/mL) | F1 | F2 | F4 | F5 | F6 | F7 | F8 |
| Batch | NB165-36 | NB165-36 | NB165-36 | NB165-36 | NB165-36 | NB165-36 | NB165-36 |
| LD-Tyrosine | 300.00 | 300.00 | 300.00 | 300.00 | 300.00 | 300.00 | 300.00 |
| Carbidopa | 13.00 | 13.00 | 13.00 | 13.00 | 13.00 | 13.00 | 13.00 |
| monohydrate | | | | | | | |
| L-Arginine | 240.00 | 180.00 | 180.00 | 240.00 | 240.00 | 180.00 | 240.00 |
| Ethanol | 155.50 | 0.00 | 0.00 | 0.00 | 0.00 | 0.00 | 155.50 |
| DMA | 0.00 | 140.55 | 0.00 | 0.00 | 0.00 | 0.00 | 0.00 |
| PEG300 | 0.00 | 0.00 | 250.00 | 250.00 | 250.00 | 135.00 | 0.00 |
| MgCl₂ | 0.00 | 0.00 | 0.00 | 100.00 | 100.00 | 50.00 | 50.00 |
| Ascorbic acid | 5.00 | 5.00 | 5.00 | 5.00 | 5.00 | 5.00 | 5.00 |
| NAC | 5.00 | 5.00 | 5.00 | 5.00 | 5.00 | 5.00 | 5.00 |
| WFI q.s | 100.00 | 100.00 | 100.00 | 100.00 | 100.00 | 100.00 | 100.00 |
| Final pH | 8.92 | 8.74 | 8.53 | 7.38 | 7.26 | 7.64 | 7.91 |

TABLE 49

| | | LD- | | | |
|---|---|---|---|---|---|
| | LD-Tyr/CD | Tyr/CD | Infusion | Infusion | Infusion |
| Treatment | Concentration | daily dose | Volume | Rate | Duration |
| Code | (mg/mL) | (mg) | (mL) | (μL/h) | (h) |
| F1 | 300/13 | 2700/117 | 9 | 485 × | 24 |
| F2 | | | | 18 h + | |
| F4 | | | | 50 × 6 h | |
| F5 | | | | | |
| F6 | | | | | |
| F7 | | | | | |
| F8 | | | | | |
| S1 | 0 | 0 | | | |

### Results

Based on the histopathological results, it can be concluded that all tested formulations were well tolerated following 24 hr SC infusion at a volume of 9 mL. In view of additional chemical experiments (stability, etc.) it was decided to focus on formulations F1 and F4, where it is noted that F4 demonstrated better local tolerance than F1.

### Experimental Example 21—Local Tolerability Studies of Formulations Comprising PEG 300 and/or Ethanol, Following 24 h SC Administration in Domestic Pigs

The local tolerability and pathology of injection sites following 24 h of SC administration in domestic pigs of the formulations and vehicles detailed in Table 50 according to the dosing regimen as detailed in Table 51 was tested.

TABLE 50

| Ingredient | Test Item | | |
|---|---|---|---|
| (mg/mL) | F1 | F2 | F3 |
| LD-Tyrosine | 300.00 | 300.00 | 300.00 |
| Carbidopa | 13.00 | 13.00 | 13.00 |
| monohydrate | | | |
| L-Arginine | 200.00 | 200.00 | 200.00 |
| Ethanol | 116.64 | 0.00 | 116.64 |
| PEG300 | 0.00 | 112.50 | 56.25 |
| NAC | 10.00 | 10.00 | 10.00 |
| WFI q.s | 100.00 | 100.00 | 100.00 |
| Final pH | 8.53 | 8.57 | 8.59 |

TABLE 51

| | | LD- | | | |
|---|---|---|---|---|---|
| | | Tyr/CD | | | |
| | LD-Tyr/CD | daily | Infusion | Infusion | Infusion |
| Treatment | Concentration | dose | Volume | Rate | Duration |
| Code | (mg/mL) | (mg) | (mL) | (μL/h) | (h) |
| F1 | 300/13 | 2700/117 | 9 | 485 × | 24 |
| F2 | | | | 18 h + | |
| F3 | | | | 50 × 6h | |
| S1 | 0 | 0 | | | |

### Results

Infusion sites were examined for any local reaction immediately after pump removal. Any dermal reaction (e.g. erythema, edema/swelling) was recorded and scored according to a 5-point Draize scale for scoring skin irritation as provided in Table 31.

No significant local skin reactions were observed in all treated sites. The reactions that were observed immediately following pump removal, were very slight to moderate (grade 1-3) erythema or edema, as shown in Table 52.

TABLE 52
Incidence of Local Reactions at the Infusion
Site Immediately following Pumps Removal

| | Erythema and eschar | | | | | Edema formation | | | | |
|---|---|---|---|---|---|---|---|---|---|---|
| | formation score | | | | | score | | | | |
| Test Item | 0 | 1 | 2 | 3 | 4 | 0 | 1 | 2 | 3 | 4 |
| F1 | 8 | 2 | 0 | 0 | 0 | 2 | 4 | 4 | 0 | 0 |
| (n = 10) | | | | | | | | | | |
| F2 | 10 | 0 | 0 | 0 | 0 | 3 | 4 | 3 | 0 | 0 |
| (n = 10) | | | | | | | | | | |
| F3 | 7 | 2 | 0 | 1 | 0 | 3 | 3 | 4 | 0 | 0 |
| (n = 10) | | | | | | | | | | |

Further, based on the histopathological results and under the conditions of this study, it can be concluded that all three formulations (F1-F3) were well tolerated following 24 hr SC infusion at a volume of 9 mL.

### Experimental Example 22—Pharmacokinetics (PK) of Levodopa (LD) Following SC Administration of LD-Tyr in a Formulation Comprising Ethanol

The objective of this study is to determine the pharmacokinetics (PK) of LD-Tyr following an 18-hr continuous subcutaneous (SC) administration in domestic pigs of a formulation comprising 30& LD-Tyr and ethanol. Table 53 provides the LD-Tyr formulation tested in this study.

TABLE 53

| | Nominal |
|---|---|
| Formulation | concentration |
| Composition | (mg/ml) |
| LD-Tyrosine | 300.00 |
| Carbidopa | 13.00 |
| L-Arginine | 240.00 |
| EtOH | 155.50 |
| NAC | 10.00 |
| WFI | q.s |

Animals were dosed with 2700 or 5400 mg of LD-Tyr in the formulation of Table 53 in an 18-hr continuous subcutaneous administration in an infusion volume of 9 or 18 ml (9 mL×2 sites).

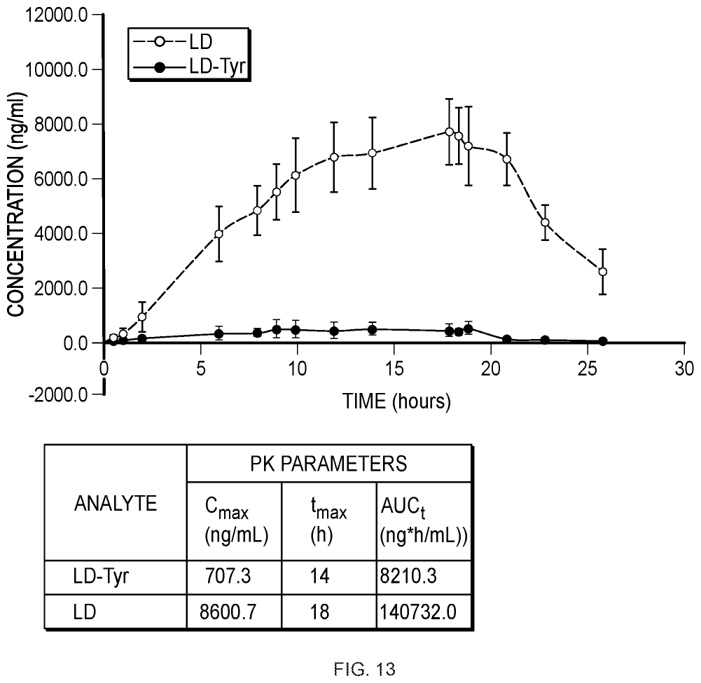

FIG. 13 shows the composite mean plasma LD-Tyr and LD concentration-versus-time profiles for the 2700 mg dosing regimen. PK parameters are also shown.

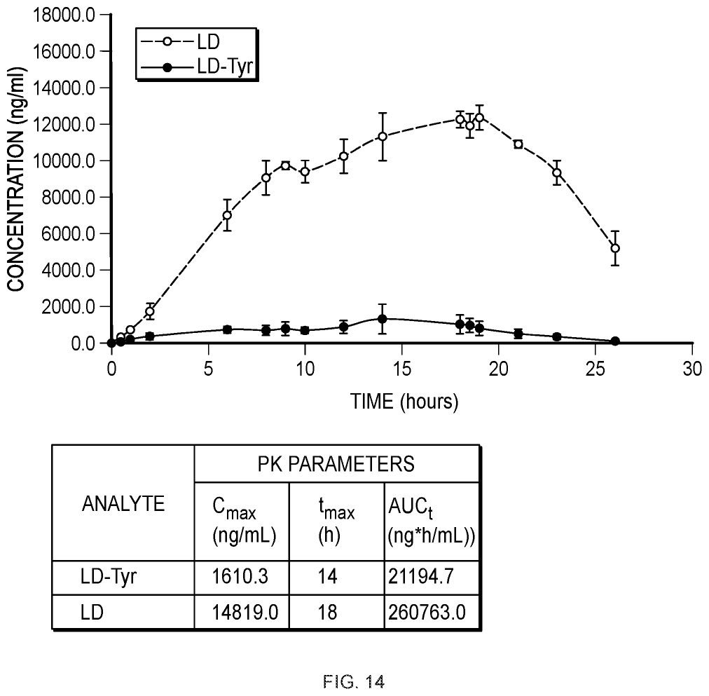

FIG. 14 shows the composite mean plasma LD-Tyr and LD concentration-versus-time profiles for the 5400 mg dosing regimen. PK parameters are also shown.

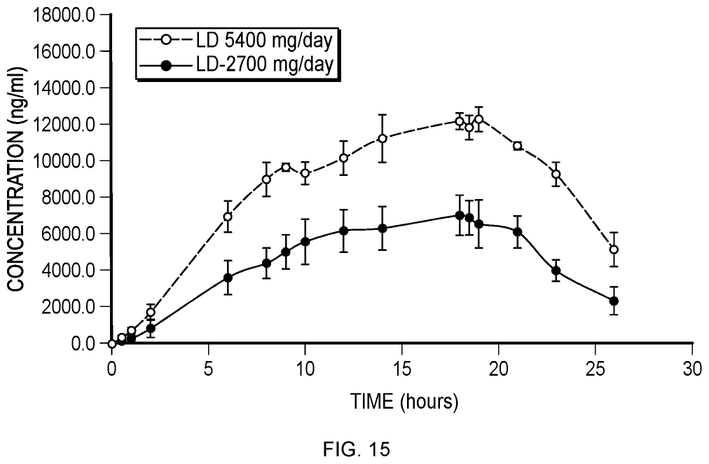

FIG. 15 shows the composite mean plasma LD concentration-versus-time profiles for the 2700 and 5400 mg dosing regimens.

Table 54 provides a summary of PK results. The results presented in FIG. 15, as well as in Table 54, show the dose proportionality of the LD-Tyr.

TABLE 54

| | | | LD-Tyr/ | | | | |
|---|---|---|---|---|---|---|---|
| | Mean | Number | CD Daily | | PK parameters | | |
| Test | Body | of | dose | | Cₘₐₓ | tₘₐₓ | AUCₜ |
| Item | Weight | animals* | (mg) | Analyte | (ng/mL) | (h) | (ng*h/mL) |
| LD- | 45.67 ± 1.04 | n = 5 | 2700/117 | LD-Tyr | 707.3 ± 282.7 | 14 | 8210.3 ± 4117.7 |
| Tyr | | | | LD | 8600.7 ± 1212.4 | 18 | 140732.0 ± 16821.7 |
| | 45.65 ± 0.77 | n = 3 | 5400/234 | LD-Tyr | 1610.3 ± 901.5 | 14 | 21194.7 ± 8567.5 |
| | | | | LD | 14819.0 ± 591.2 | 18 | 260763.0 ± 7323.3 |

### Experimental Example 23

In order to optimize antioxidants in LD-Tyr formulations, the following formulations were prepared, and assessed analytically.

TABLE 55
Preliminary Formulations

| Ingredient | | | | |
|---|---|---|---|---|
| (mg/ml) | Control | F1 | F2 | F3 |
| LD-Tyrosine | 300 | 300 | 300 | 300 |
| Carbidopa | 13 | 13 | 13 | 13 |
| NAC | 10 | 10 | 20 | 0 |
| Ascorbic | 0 | 10 | 0 | 20 |
| L-Arginine | 240 | 240 | 240 | 240 |
| Ethanol | 155.5 | 155.5 | 155.5 | 155.5 |

TABLE 56
Analytical Results

| | T = 0 | | 1 month at 2-8° C. | | 1 month at 25° C. | |
|---|---|---|---|---|---|---|
| | Main | | Main | | Main | |
| | Degradant | Total CD | Degradant | Total CD | Degradant | Total CD |
| Antioxidants | (MD) | impurities | (MD) | impurities | (MD) | impurities |
| 10 mg/ml NAC | 0.12 | 0.52 | 0.52 | 0.87 | 0.93 | 1.34 |
| (Control) | | | | | | |
| 10 NAC mg/ml + | <0.05 | <0.05 (0.02) | <0.05 (0.02) | 0.33 | 0.07 | 0.49 |
| 10 Asc mg/ml | | | | | | |
| 20 mg/ml NAC | 0.13 | 0.40 | 0.40 | 0.72 | 0.80 | 1.22 |
| 20 mg/ml Asc | 0.33 | 0.86 | 0.86 | 1.66 | 1.48 | 3.16 |

The results presented in Table 56 show that neither one of the antioxidants alone prevents impurities as well as the combination of them both does. Accordingly, final amount of each one of ascorbic acid and NAC will be between about 0.1% and about.

The following additional formulations detailed in Table 57 were also prepared, and their analytical values at time 0 were measured using two analytical methods, as detailed in Tables 58a and 58b.

TABLE 57

| Ingredient | | | | | |
|---|---|---|---|---|---|
| (mg/ml) | F1 | F2 | F3 | F4 | F5 |
| LD-Tyrosine | 300 | 300 | 300 | 300 | 300 |
| Carbidopa | 13 | 13 | 13 | 13 | 13 |
| NAC | 10 | 10 | 10 | 10 | 10 |
| Ascorbic acid | 0 | 2.5 | 5 | 7.5 | 10 |
| L-Arginine | 240 | 240 | 240 | 240 | 240 |
| Ethanol | 155.5 | 155.5 | 155.5 | 155.5 | 155.5 |

TABLE 58a

T = 0

| Concentration | | Total CD |
|---|---|---|
| (mg/ml) | MD | impurities |
| 10 NAC (Control) | 0.07 | 0.38 |
| 2.5 Asc + 10 NAC | ND | 0.30 |
| 5 Asc + 10 NAC | ND | 0.30 |
| 7.5 Asc + 10 NAC | ND | 0.30 |
| 10 Asc + 10 NAC | ND | 0.30 |

TABLE 58b

T = 0

| Concentration | | Total CD |
|---|---|---|
| (mg/ml) | MD | impurities |
| 10 NAC (Control) | 0.07 | 0.63 |
| 2.5 Asc + 10 NAC | ND | 0.55 |
| 5 Asc + 10 NAC | ND | 0.43 |
| 7.5 Asc + 10 NAC | ND | 0.44 |
| 10 Asc + 10 NAC | ND | 0.44 |

Once the amount of the ascorbic acid is optimized, the amount of NAC will be optimized as well. Contemplated formulations used to optimize the amount of NAC are provided in Table 59 below:

| Ingredient | | | | | |
|---|---|---|---|---|---|
| (mg/ml) | F1 | F2 | F3 | F4 | F5 |
| LD-Tyrosine | 300 | 300 | 300 | 300 | 300 |
| Carbidopa | 13 | 13 | 13 | 13 | 13 |
| NAC | 0 | 2.5 | 5 | 7.5 | 10 |
| Ascorbic acid | 0-10* | 0-10* | 0-10* | 0-10* | 0-10* |
| L-Arginine | 240 | 240 | 240 | 240 | 240 |
| Ethanol | 155.5 | 155.5 | 155.5 | 155.5 | 155.5 |

*to be set according to optimization of ascorbic acid amount

### EQUIVALENTS

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. All numbers expressing quantities of ingredients, reaction conditions, and so forth used in the specification are to be understood as being modified in all instances by the term “about”, even if the term “about” is not specifically recited in respect to any of the disclosed embodiments.

### INCORPORATION BY REFERENCE

The entire contents of all patents, published patent applications, websites, and other references referred to herein, are hereby expressly incorporated herein in their entireties by reference.

What is claimed is:

1. A levodopa amino acid complex represented by the following formula (I) or (III) or a pharmaceutically acceptable salt thereof: 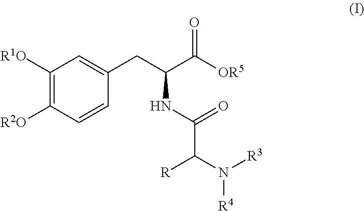
wherein R is an amino acid side chain that may be substituted;
R¹ and R² may be the same or different, and are each independently a hydrogen atom, a C₁-C₆ alkyl, a C₁-C₆ alkanoyl, a phosphono, a sulfino, or a glycosyl that may be substituted, provided that R¹ and R² are not hydrogen atom at the same time;
R³ and R⁴ may be the same or different, and are each independently a hydrogen atom or a C₁-C₆ alkyl; and
R⁵ is a hydrogen atom; or 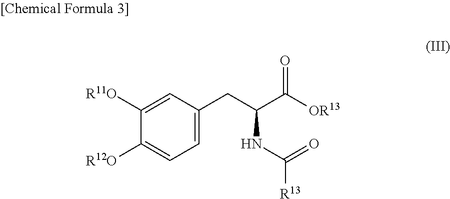
wherein R¹¹ and R¹² are the same or different and are each a hydrogen, an alkyl, an alkanoyl, a P(═O)(OH)₂, S(═O)(OH) or a glycosyl that may be substituted;
R¹³ is an alkyl that may be substituted, —R¹⁵—O—R¹⁶ or a 5-membered heterocyclyl containing at least one nitrogen atom, wherein R¹⁵ is an alkylene, and R¹⁶ is a hydrogen, an alkyl, P(═O)(OH)₂, S(═O)(OH), or a glycosyl that may be substituted; and
R¹⁴ is a hydrogen or an alkyl;
provided that following compounds are excluded;
(2S)-2-[(2-aminoacetyl)amino]-3-(3,4-diacetyloxyphenyl)propanoic acid,
(2S)-2-[[(2S)-2-amino-6-[(2-chlorophenyl)methoxycarbonylamino]hexanoyl]amino]-3-(3,4-dimethoxyphenyl)propanoic acid,
(2S)-2-[[(2S)-2-amino-3-(3,4-dihydroxyphenyl)propanoyl]amino]-3-(4-hydroxy-3-methoxyphenyl)propanoic acid,
(2S)-2-[[(2S)-2-amino-3-phenylpropanoyl]amino]-3-(3,4-dimethoxyphenyl)propanoic acid,
(2S)-2-[[(2R)-2-amino-3-phenylpropanoyl]amino]-3-(3,4-diacetyloxyphenyl)propanoic acid, and
(2S)-2-[[(2S)-2-amino-5-methoxy-5-oxopentanoyl]amino]-3-(3,4-dimethoxyphenyl)propanoic acid.

2. The levodopa amino acid complex according to claim 1 or a pharmaceutically acceptable salt thereof, wherein
R³, R⁴ and R⁵ are hydrogen atoms, and
R¹ and R² are the same or different, and are each a hydrogen atom, an acetyl or a phosphono, provided that R¹ and R² are not hydrogen atom at the same time.

3. The levodopa amino acid complex according to claim 1 or 2 or a pharmaceutically acceptable salt thereof, wherein
R³, R⁴ and R⁵ are hydrogen atoms,
R¹ is a hydrogen atom, and
R² is a phosphono.

4. The levodopa amino acid complex according to any one of claims 1-3 or a pharmaceutically acceptable salt thereof, wherein
an amino acid of the amino acid side chain is a glutamic acid, valine, alanine, lysine, 3,4-dihydroxyphenylalanine or tyrosine.

5. The levodopa amino acid complex selected from the group consisting of:
(2S)-2-[[(2S)-2-amino-3-phosphonooxypropanoyl]amino]-3-(3,4-dihydroxyphenyl)propanoic acid,
(2S)-2-[[(2S)-2-amino-3-(4-phosphonooxyphenyl)propanoyl]amino]-3-(3,4-dihydroxyphenyl)propanoic acid,
(2S)-2-amino-5-[[(1S)-1-carboxy-2-(3,4-diacetoxyphenyl)ethyl]amino]-5-oxo-pentanoic acid,
(2S)-3-(3,4-dihydroxyphenyl)-2-[(2-methyl-2-phosphonooxypropanoyl)amino]propanoic acid, and
(2S)-2-[[(2S)-2-amino-3-[4-[(2S,3R,4S,5S,6R)-3,4,5-trihydroxy-6-(hydroxymethyl)oxan-2-yl]oxyphenyl]propanoyl]amino]-3-(3,4-dihydroxyphenyl)propanoic acid.

6. A liquid pharmaceutical composition comprising the levodopa amino acid complex according to any one of claims 1-5 or a pharmaceutically acceptable salt thereof as an active ingredient.

7. A therapeutic agent for a neurodegenerative disease and/or a disease or symptom caused by a decrease in dopamine concentration in the brain, the therapeutic agent comprising the levodopa amino acid complex according to any one of claims 1-5 or a pharmaceutically acceptable salt thereof as an active ingredient.

8. The therapeutic agent according to claim 7, wherein
the neurodegenerative disease and/or the disease or symptom caused by a decrease in dopamine concentration in the brain is Parkinson's disease.

9. A liquid pharmaceutical composition comprising:
a levodopa-tyrosine conjugate (LD-Tyr) of formula (II): 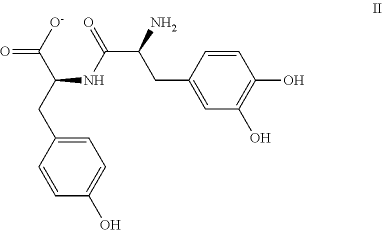 [SMILES: N[C@@H](Cc1ccc(O)c(O)c1)C(=O)N[C@@H](Cc1ccc(O)cc1)C(=O)[O-]]
an enantiomer, diastereomer, racemate, ion, zwitterion, pharmaceutically acceptable salt thereof, or any combination thereof; and
a stabilizer.

10. The liquid pharmaceutical composition of claim 9, comprising between about 10 to about 45% w/v of the LD-Tyr, enantiomer, diastereomer, racemate, ion, zwitterion, pharmaceutically acceptable salt thereof, or any combination thereof.

11. The liquid pharmaceutical composition of claim 9, comprising at least about 30% w/v of the LD-Tyr, enantiomer, diastereomer, racemate, ion, zwitterion, pharmaceutically acceptable salt thereof, or any combination thereof.

12. The liquid pharmaceutical composition of claim 10 or claim 11, comprising between about 30 to about 45% w/v of the LD-Tyr, enantiomer, diastereomer, racemate, ion, zwitterion, pharmaceutically acceptable salt thereof, or any combination thereof.

13. The liquid pharmaceutical composition according to any one of claims 9-12, wherein the stabilizer is present in an amount of about 0.1 to about 30% w/v.

14. The liquid pharmaceutical composition according to any one of claims 9-13, wherein the stabilizer comprises a base.

15. The liquid pharmaceutical composition according to claim 14, wherein the base is selected from the group consisting of arginine, NaOH, NH₄OH, tris(hydroxymethyl)aminomethane (TRIS), ethylendiamine, diethylamine, ethanolamine, diethanolamine, meglumine, and any combination thereof.

16. The liquid pharmaceutical composition according to claim 15, wherein the base is selected from the group consisting of arginine, NH₄OH, ethylendiamine, diethylamine, ethanolamine, diethanolamine, meglumine, and any combination thereof.

17. The liquid pharmaceutical composition according to claim 16, wherein the base is selected from the group consisting of L-Arg, diethylamine, and a combination thereof.

18. The liquid pharmaceutical composition according to claim 17, wherein the base is selected from the group consisting of L-Arg, ethanolamine, and a combination thereof.

19. The liquid pharmaceutical composition according to any one of claims 14-18, wherein said liquid pharmaceutical composition comprises between about 0.1 to about 30% w/v of the base.

20. The liquid pharmaceutical composition according to claim 19, wherein said liquid pharmaceutical composition comprises between about 1.5 to about 20% w/v of the base.

21. The liquid pharmaceutical composition according to any one of claims 9-20, wherein the liquid pharmaceutical composition has a pH in the range of between about 5 to about 10 at about 25° C.

22. The liquid pharmaceutical composition of claim 21, wherein the liquid pharmaceutical composition has a pH in the range of between about 8 to about 10 at about 25° C.

23. The liquid pharmaceutical composition according to claim 22, wherein the liquid pharmaceutical composition has a pH in the range of between about 8 to about 9 at about 25° C.

24. The liquid pharmaceutical composition according to any one of claims 9-23, further comprising a decarboxylase inhibitor.

25. The liquid pharmaceutical composition according to claim 24, wherein the decarboxylase inhibitor is carbidopa.

26. The liquid pharmaceutical composition according to any one of claims 24 or 25, wherein said liquid pharmaceutical composition comprises between about 0.25 to about 1.5% w/v of the decarboxylase inhibitor.

27. The liquid pharmaceutical composition according to any one of claims 9-26, further comprising an antioxidant or a combination of two or more antioxidants.

28. The liquid pharmaceutical composition according to claim 27, wherein the antioxidant is each independently selected from the group consisting of ascorbic acid or a salt thereof, a cysteine, a bisulfite or a salt thereof, glutathione, a tyrosinase inhibitor, a Cu²⁺ chelator, and any combination thereof.

29. The liquid pharmaceutical composition according to claim 28, wherein the cysteine is N-acetyl cysteine (NAC).

30. The liquid pharmaceutical composition according to any one of claims 27-29, wherein the antioxidant is a combination of ascorbic acid and NAC.

31. The liquid pharmaceutical composition according to any one of claims 27-30, wherein said liquid pharmaceutical composition comprises between about 0.05 to about 1.5% w/v of the antioxidant or the combination of antioxidants.

32. The liquid pharmaceutical composition according to any one of claims 1-31 further comprising at least one of: a catechol-O-methyltransferase (COMT) inhibitor, a monoamine oxidase (MAO) inhibitor, a surfactant, a buffer, an acid, a solvent, and any combination thereof.

33. The liquid pharmaceutical composition according to claim 32, wherein the buffer is TRIS.

34. The liquid pharmaceutical composition according to any one of claims 32-33, wherein said liquid pharmaceutical composition comprises between about 5.0 to about 40.0% w/v of the buffer.

35. The liquid pharmaceutical composition according to any one of claims 1-34, wherein the stabilizer comprises polyethylene glycol.

36. The liquid pharmaceutical composition according to any one of claims 1-35, wherein the liquid pharmaceutical composition comprises less than about 1.5% w/v LD-Tyr-diketopiperazine after two weeks at 2-8° C.

37. The liquid pharmaceutical composition according to claim 36, wherein the liquid pharmaceutical composition comprises less than about 0.8% w/v LD-Tyr-diketopiperazine after two weeks at 2-8° C.

38. The liquid pharmaceutical composition according to any one of claims 1-36, wherein the liquid pharmaceutical composition comprises less than about 5.0% w/v LD-Tyr-diketopiperazine after two weeks at 25° C.

39. The liquid pharmaceutical composition according to claim 38, wherein the liquid pharmaceutical composition comprises no more than about 4.0% w/v LD-Tyr-diketopiperazine after two weeks at 25° C.

40. A method of treating neurodegenerative conditions and/or conditions characterized by reduced levels of dopamine in the brain, wherein the method comprises administering an effective amount of a liquid pharmaceutical composition according to any one of claims 1-39.

41. The method according to claim 40, wherein the neurodegenerative condition is Parkinson's disease.

42. The method according to claim 40 or claim 41, wherein the liquid pharmaceutical composition is administered concomitantly to the patient with an additional active ingredient.

43. The method according to claim 42, wherein the additional active ingredient is selected from the group consisting of a decarboxylase inhibitor, a COMT inhibitor, a MAO inhibitor, and any combination thereof.

44. The method according to any one of claims 40-43, wherein the liquid pharmaceutical composition is administered substantially continuously to the patient.

45. The method according to any one of claims 40-44, wherein the liquid pharmaceutical composition is administered subcutaneously.

46. The liquid pharmaceutical composition according to any one of claims 1-39 for use in treating neurodegenerative conditions and/or conditions characterized by reduced levels of dopamine in the brain.

47. The liquid pharmaceutical composition according to claim 46, wherein the neurodegenerative condition is Parkinson's disease.

48. The liquid pharmaceutical composition according to claim 46 or claim 47, wherein the liquid pharmaceutical composition is administered concomitantly to the patient with an additional active ingredient.

49. The liquid pharmaceutical composition according to claim 48, wherein the additional active ingredient is selected from the group consisting of a decarboxylase inhibitor, a COMT inhibitor, a MAO inhibitor, and any combination thereof.

50. The liquid pharmaceutical composition according to any one of claims 46-49, wherein the liquid pharmaceutical composition is administered substantially continuously to the patient.

51. The liquid pharmaceutical composition according to any one of claims 46-50, wherein the liquid pharmaceutical composition is administered subcutaneously.
